(19) United States
(12) Patent Application Publication
(10) Pub. No.: US 2014/0208832 A1
(43) Pub. Date: Jul. 31, 2014

(54) Methods and Apparatus for Flow-Controlled Wetting

(71) Applicant: The University of British Columbia, Vancuuver (CA)

(72) Inventors: Carl Lars Genghis Hansen, Vancouver (CA); Kaston K. Leung, Vancouver (CA); Timothy Leaver, Delta (CA); Hans Zahn, Vancouver (CA)

(73) Assignee: The University of British Columbia, Vancouver (CA)

(21) Appl. No.: 14/346,240

(22) PCT Filed: Sep. 28, 2012
(86) PCT No.: PCT/CA2012/050684

§ 371 Date: Mar. 20, 2014

Related U.S. Application Data
(60) Provisional application No. 61/541,916, filed on Sep. 30, 2011.

(51) Int. Cl. G01N 1/28; G01N 33/00

(52) U.S. Cl. CPC G01N 1/28; G01N 33/00
USPC 73/53.01; 137/1; 137/594; 239/548; 427/8

(57) ABSTRACT

Methods of determining a first position at which a dispersed phase droplet wets a surface of a channel are provided herein. The methods include immersing the dispersed phase droplet in a continuous phase fluid, wherein the continuous phase fluid is immiscible with the dispersed phase droplet, subsequently flowing the dispersed phase droplet in the continuous phase through the channel at a dispersed phase droplet velocity, wherein the dispersed phase droplet is separated from the surface by a film of the continuous phase fluid having a film thickness, and reducing the film thickness to rupture the film at the first position, wherein the droplet wets the surface at the first position.

### CROSS-REFERENCE TO RELATED APPLICATIONS

This application claims the priority benefit of U.S. provisional application Ser. No. 61/541,916 filed 30 Sep. 2011, which is incorporated herein by reference.

### BACKGROUND OF THE INVENTION

1. Field of Invention

This invention relates to microfluidic devices. In particular, the invention relates to droplet-based microfluidic devices and their uses and methods for sequential combination of reactants to a reaction mixture.

2. Description of Related Art

Microfluidic systems provide numerous advantages for biological analysis including automation, enhanced reaction efficiency in small volumes, favorable mass transport properties, and the potential for scalable and cost-effective analysis of limited samples. The development of microfluidic systems over the past decade has resulted in increasingly sophisticated on-chip functionality and the emergence of two orthogonal strategies for controlling and storing fluids, based either on the use of integrated microvalves or the transport of microdroplets in an immiscible carrier phase. The development of soft lithography (1) and the extension of this method to the fabrication of integrated microvalves using Multilayer Soft Lithography (2, 3) has enabled devices with thousands of active microvalves per cm². This high level of integration enables device architectures capable of executing thousands of pre-defined “unit cell” reactions in parallel, with applications including protein crystallization (4, 5), protein interaction studies (6, 7), single cell analysis (8-11), cell culture (12, 13), and genomics (14). More recently, two-phase microdroplet systems have been developed which are ideally suited to high-speed serial analysis and have been used in high-throughput screening applications (15-17) and sample preparation for genomics (18).

Such devices have clearly demonstrated the potential impact of microfluidics in multiple fields of biological research, yet the use of microfluidic devices largely remains the purview of microfluidics-focused laboratories. This is primarily due to the fact that these devices require specialized training and equipment to fabricate, and are typically “hard-wired” for a specific fluid handling tasks, necessitating a custom design and fabrication cycle for each application or change in assay protocol. Unfortunately, such design iterations are inaccessible to the typical biology laboratory. A general, flexible, and user-programmable microfluidic platform would do much to remove this barrier to widespread adoption by the general scientific community.

Electrowetting-on-dielectric systems, which manipulate droplets on arrays of electrodes, achieve a high degree of programmability and flexibility by allowing for control over multiple droplets simultaneously, including the ability to merge and split droplets of defined volumes (19). Programmable devices that use valves to reconfigure a grid of nodes through which fluids can be routed have also been demonstrated (20-22). However, such systems have thus far implemented a limited number of parallel reactions, and have a relatively low resolution of formulation.

The importance of cell-to-cell heterogeneity in all biological systems and the amenability of microfluidics to single cell analysis makes the manipulation of single cells an important capability that many potential users of a programmable microfluidic device would benefit from. However, this capability has yet to be integrated into existing programmable microfluidic systems.

### SUMMARY OF THE INVENTION

The invention relates to general and flexible microfluidic methods and systems that allow for programmable high-resolution formulation of nanolitre-scale solutions and elution of reaction products in an array of individually addressable storage elements. This functionality is enabled by a of a novel droplet storage strategy that uses flow-controlled wetting to position and merge a completely programmable number of droplets.

In accordance with one embodiment, there is provided a method of determining a first position at which a dispersed phase droplet wets a surface of a channel having a uniform wettability. The method includes the steps of immersing the dispersed phase droplet in a continuous phase fluid, wherein the continuous phase fluid is immiscible with the dispersed phase droplet, subsequently flowing the dispersed phase droplet in the continuous phase through the channel at a dispersed phase droplet velocity, wherein the dispersed phase droplet is separated from the surface by a film of the continuous phase fluid having a film thickness, and rupturing the film at the first position, wherein the droplet wets the surface at the first position. Rupturing the film may include reducing the dispersed phase droplet velocity to reduce the film thickness.

In accordance with another embodiment, there is provided a method of determining a first position at which a dispersed phase droplet wets a surface of a channel. The method includes the steps of immersing the dispersed phase droplet in a continuous phase fluid, wherein the continuous phase fluid is immiscible with the dispersed phase droplet, subsequently flowing the dispersed phase in the continuous phase through the channel at a dispersed phase droplet velocity, wherein the dispersed phase droplet is separated from the surface by a film of the continuous phase fluid having a film thickness, and reducing the film thickness to rupture the film at the first position, wherein the droplet wets the surface at the first position. Rupturing the film may include reducing the dispersed phase droplet velocity to reduce the film thickness. Reducing the film thickness may include removing a portion of the continuous phase fluid from the channel as the dispersed phase droplet approaches the first position.

In accordance with another embodiment, there is provided a method of combining a plurality of dispersed phase droplets. The method includes the steps of a) maintaining a first dispersed phase droplet wetted to a surface of a channel at a first position; b) causing a second dispersed phase droplet to wet the surface of the channel at the first position according to methods described herein; and c) contacting the first dispersed phase droplet with the second dispersed phase droplet for a period sufficient for the first dispersed phase droplet and the second dispersed phase droplet to combine. The method may further include the introduction of additional droplets to the channel, and subsequent merging with previously combined droplets.

The first, second, and additional droplets may contain the same solution, or may contain different solutions. Each droplet may contain different components of a chemical reaction. Accordingly, the method may be used for the formulation and execution of a multi-step reaction by sequential addition of dispersed phase droplets.

In accordance with another embodiment, there is provided a method of removing a first portion of a dispersed phase immersed in a continuous phase fluid, wherein the continuous phase fluid is immiscible with the dispersed phase, from a dispersed phase retaining chamber operably configured to retain the first portion provided that the volume of the first portion is less than the volume of the chamber. The method includes the steps of: a) immersing a dispersed phase droplet in the continuous phase fluid, wherein the continuous phase fluid is immiscible with the dispersed phase droplet, to form a second portion of the dispersed phase; b) flowing the second portion of the dispersed phase into the dispersed phase retaining chamber, wherein the total volume of the dispersed phase portions in the retaining chamber exceeds the volume of the dispersed phase retaining chamber; c) contacting the first dispersed phase portion with the second dispersed phase portion for a period sufficient for the first dispersed portion and second dispersed phase portion to combine to form an elution stream encapsulated in the continuous phase fluid; and d) flowing the elution stream through a dispersed phase retaining chamber exit.

In accordance with another embodiment, there is provided a microfluidic device for reducing the thickness of a film of a continuous phase fluid encapsulating a dispersed phase droplet, wherein the dispersed phase droplet is immiscible in the continuous phase fluid. The device includes a channel for flowing the dispersed phase droplet, and a series of sieve elements operably configured to divert a portion of the continuous phase fluid from the channel to reduce the thickness of the film, wherein each sieve element has a diameter smaller than the diameter of the dispersed phase droplet. The sieve elements may be aligned generally perpendicular to the channel. The device may further include a dispersed phase retaining chamber in fluid communication with the channel for receiving the dispersed phase droplet from the channel. The sieve elements may be operably configured to divert the portion of the continuous phase fluid from the channel prior to reaching the dispersed phase retaining chamber. The device may further include a bypass channel in fluid communication with the series of sieve elements, wherein the bypass channel is operably configured to receive the portion and maintain the portion outside the retaining chamber.

In accordance with another embodiment, there is provided a microfluidic device for reducing a velocity of a dispersed phase droplet encapsulated in a continuous phase fluid, wherein the dispersed phase droplet is immiscible with the continuous phase fluid. The device includes a channel for flowing the dispersed phase droplet, and a series of sieve elements operably configured to divert a portion of the continuous phase fluid from the channel to reduce the velocity of the dispersed phase droplet, wherein each sieve element has a diameter smaller than the diameter of the dispersed phase droplet. The sieve elements may be aligned generally perpendicular to the channel. The device may further include a dispersed phase retaining chamber in fluid communication with the channel for receiving the dispersed phase droplet from the channel. The sieve elements may be operably configured to divert the portion of the continuous phase fluid from the channel prior to reaching the dispersed phase retaining chamber. The device may further include a bypass channel in fluid communication with the series of sieve elements, wherein the bypass channel is operably configured to receive the portion and maintain the portion outside the retaining chamber.

In accordance with another embodiment, there is provided a process of treating a dispersed phase droplet in a microfluidic device. The process includes the steps of a) immersing a first dispersed phase droplet in a continuous phase fluid, wherein the continuous phase fluid is immiscible with the first dispersed phase droplet, to form a first portion of a dispersed phase; b) flowing the first dispersed phase droplet into a storage element of the device with a first dispersed phase droplet velocity, wherein the storage element includes a main channel and a dispersed phase retaining chamber for receiving said dispersed phase droplet from the main channel, wherein the main channel is operably configured to reduce dispersed phase droplet velocity as said dispersed phase droplet approaches the retaining chamber, and wherein the retaining chamber is operably configured to retain said dispersed phase droplet within the storage element provided that the total volume of the dispersed phase within the retaining chamber is less than the volume of the retaining chamber, wherein the first dispersed phase droplet is separated from a surface of the storage element by a first film of the continuous phase fluid having a first film thickness; and c) rupturing the first film at a first position within the storage element, wherein the first dispersed phase droplet wets the surface at the first position. Rupturing the first film at the first position may include reducing the first film thickness to rupture the first film at the first position. Reducing the first film thickness may include reducing the first dispersed phase droplet velocity. The surface may have a uniform wettability.

The process may further include the steps of d) immersing a second dispersed phase droplet in the continuous phase fluid, wherein the continuous phase fluid is immiscible with the second dispersed phase droplet, to form a second portion of the dispersed phase; e) flowing the second dispersed phase droplet into the storage element with a second dispersed phase droplet velocity, wherein the second dispersed phase droplet is separated from the surface of the storage element by a second film of the continuous phase fluid having a second film thickness; and f) rupturing the second film at a second position within the storage element, wherein the second droplet wets the surface at the second position. The first position may be substantially the same as the second position, and the first dispersed phase droplet may be contacted with the second dispersed phase droplet for a period sufficient for first dispersed phase droplet and second dispersed phase droplet to combine.

The process may further include the steps of g) immersing a third dispersed phase droplet in the continuous phase fluid, wherein the continuous phase fluid is immiscible with the third dispersed phase droplet, to form a third portion of the dispersed phase; h) flowing the third dispersed phase droplet into the storage element with a third dispersed phase droplet velocity, wherein the third dispersed phase droplet is separated from the surface of the storage element by a third film of the continuous phase fluid having a third film thickness; and i) rupturing the third film at a third position within the storage element, wherein the third droplet wets the surface at the third position. The third position may be substantially the same as the first position, and the third dispersed phase droplet may be contacted with the first dispersed phase droplet for a period sufficient for first dispersed phase droplet and third dispersed phase droplet to combine. Alternatively, the third position may be substantially the same as the second position, and the third dispersed phase droplet may be contacted with the second dispersed phase droplet for a period sufficient for second dispersed phase droplet and third dispersed phase droplet to combine. In a further alternative, the third position may lie between the first position and the second position, and the third position may be substantially close to the first position and to the second position, wherein the third dispersed phase droplet may contact both the first dispersed phase droplet and the second dispersed phase droplet for a period sufficient for the third dispersed phase droplet to combine with the first dispersed phase droplet and the second dispersed phase droplet.

The process may further include the steps of j) immersing a fourth dispersed phase droplet in the continuous phase fluid, wherein the continuous phase fluid is immiscible with the fourth dispersed phase droplet, to form a fourth portion of the dispersed phase; k) flowing the fourth dispersed phase droplet into the dispersed phase retaining chamber, wherein the total volume of the dispersed phase within the storage element exceeds the volume of the dispersed phase retaining chamber; l) contacting the dispersed phase droplets within storage element with the fourth dispersed phase droplet for a period sufficient for the fourth dispersed phase droplet and the dispersed phase droplets to combine to form an elution stream encapsulated in the carrier fluid; and m) flowing the elution stream through a dispersed phase retaining chamber exit.

One of skill in the art will understand that the number of dispersed phase droplets that may be sequentially flowed into the storage element and merged with previously immobilized dispersed phase droplet is limited only by the volume of the retaining chamber.

In accordance with another embodiment, there is provided a microfluidic system for storing and processing dispersed phase droplets. The system includes an array of at least two parallel independently addressable storage elements as described herein, wherein each storage element has a main channel and a dispersed phase retaining chamber for receiving at least one of said dispersed phase droplets from the main channel. The at least one of said dispersed phase droplets forms a portion of a dispersed phase within the storage element. The main channel is operably configured to reduce the velocity of the at least one of said dispersed phase droplets as the at least one of said dispersed phase droplets approaches the dispersed phase retaining chamber. The dispersed phase retaining chamber is operably configured to retain the at least one of said dispersed phase droplets within the storage element provided that the total volume of the dispersed phase within the dispersed phase retaining chamber is less than the volume of the retaining chamber. The system further includes an inlet channel shared by the at least two storage elements for flowing the at least one of said dispersed phase droplets to a selected storage element, and an elution channel shared by the at least two storage elements for flowing the dispersed phase from the selected storage element.

In accordance with another embodiment, there is provided a method of determining a first position at which a dispersed phase droplet wets a dispersed phase wetting surface of a microfluidic device having a uniform wettability. The method includes the steps of immersing the dispersed phase droplet in a continuous phase fluid, wherein the continuous phase fluid is immiscible with the dispersed phase droplet; subsequently flowing the dispersed phase droplet immersed through the microfluidic device at a dispersed phase droplet velocity, wherein the dispersed phase droplet is separated from the surface of the conduit by a film of the carrier liquid having a film thickness; and reducing the film thickness to rupture the film at the first position.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a schematic drawing of a microfluidic channel for use with a method according to an embodiment of the invention.

FIG. 2 is a schematic drawing of a microfluidic channel for use with a method according to an embodiment of the invention.

FIG. 3 is a schematic drawing of a dispersed phase droplet storage element according to an embodiment of the invention.

FIG. 4 is a schematic drawing of a two-dimensional array of storage elements as depicted in FIG. 3.

FIG. 5 is a schematic drawing of cell sorting module for use with array depicted in FIG. 4.

FIG. 6 is a time course of images of a single pump increment of red food dye dispensed into a flowing carrier fluid stream for transport to the storage chamber array. A four-step peristaltic pump cycle advances the aqueous stream and valve actuation pinches off a droplet. Scale bar is 500 μm.

FIG. 7 is a time course of images of droplet merging and immobilization at the critical incoming velocity. Droplets are decelerated to the droplet wetting velocity immediately before reaching the storage chamber and are pulled into it by surface tension. Scale bar is 100 μm.

FIG. 8 is a time course of images of droplet merging and immobilization below the critical incoming velocity. Droplets are decelerated to the droplet wetting velocity upstream of the storage chamber. Multiple droplets merge before the combined droplet reaches the chamber and is pulled into it by surface tension. Scale bar is 100 μm.

FIG. 9 is a time course of images of droplet merging and immobilization above the critical incoming velocity. Droplets are not decelerated to the droplet wetting velocity and freely flow to the storage chamber ceiling where they merge. Focus has been shifted up vertically in the last image to show the merged droplet positioned at the ceiling. Scale bar is 100 μm.

FIG. 10A is a micrograph of a 2.7 nL stored droplet of water stored in a chamber of a microfluidic device according to the embodiment of the invention depicted in FIG. 3.

FIG. 10B is a micrograph of a 2.7 nL stored droplet of 0.1% Tween 20 surfactant in water stored in a chamber of a microfluidic device according to the embodiment of the invention depicted in FIG. 3. The surfactant enhances wetting onto the device surface, resulting in a reduced contact angle relative to water alone.

FIG. 11 is a schematic depiction of a finite element simulation of the flow velocity through the storage element according to the embodiment of the invention depicted in FIG. 3 at a height of 2.5 μm.

FIG. 12 is a schematic depiction of a finite element simulation of the flow velocity through the storage element according to the embodiment of the invention depicted in FIG. 3 on the vertical plane through the centre of the storage element.

FIG. 13 is an optical micrograph showing the elution of a chamber according to the embodiment of the invention depicted in FIG. 1

FIG. 14 is an image of a 3-axis robotic to which the device depicted in FIG. 4 is mounted to allow for computer-controlled positioning of the elution nozzle into microwell plates.

FIG. 15 is a graph of mean fluorescent intensity as a function of dye concentration.

FIG. 16 is a scatter plot showing the measured volumes of stored droplets in 9 different chambers loaded from each of 8 reagent inlets.

FIG. 17 is an optical micrograph of a microfluidic display demonstrating the addressability and programmability of according to an embodiment of the invention. Stored droplets are composed of 300 metered droplets arranged in letters with a two-fold dilution series of dye from top to bottom of each letter.

FIG. 18A is a fluorescent micrograph of a stored droplet of FITC-labeled BSA using 100 nM BSA+0.1% Tween 20, FC-40+PFO.

FIG. 18B is a fluorescent micrograph of a stored droplet of FITC-labeled BSA using 1 uM BSA, FC-40+PFO.

FIG. 18C is a fluorescent micrograph of a stored droplet of FITC-labeled BSA using 1 uM BSA+0.1% Tween 20, FC-40+OEG fluorosurfactant.

FIG. 18D is a fluorescent micrograph of a stored droplet of FITC-labeled BSA using 1 uM BSA, FC-40+OEG fluorosurfactant.

FIG. 19 shows an endpoint fluorescent image of the droplet array following 40 cycles of PCR.

FIG. 20 shows real time amplification curves for each droplet in the array depicted in FIG. 19.

FIG. 21 is a graph of the mean C_{T} values from reactions depicted in FIG. 14 at each template dilution.

FIG. 22 is an endpoint fluorescent image following 40 cycles of PCR of droplets loaded with either template (1476 genome copies per droplet) or buffer in a checkerboard pattern.

FIG. 23 is a graph showing the fold concentration difference of template in eluted pairs of droplets containing amplified template and water.

FIG. 24 is an image comprised of overlaid brightfield and fluorescence micrographs of a single red fluorescent protein-expressing salmonella bacterium in a stored droplet.

FIG. 25 is a graph of integrated green fluorescent protein fluorescence over time of cultures seeded in storage elements with a single sorted bacterium.

FIG. 26 is a graph of integrated red fluorescent protein fluorescence over time of cultures seeded in storage elements with a single sorted bacterium.

FIG. 27 is an image comprised of overlaid GFP and RFP-channel confocal scans of all cultures a stored droplet array.

FIG. 28 is a scatterplot of normalized endpoint fluorescence intensity in GFP and RFP channels for co-cultures seed with different numbers of both strains.

FIG. 29 is a graph depicting real time qPCR curves for qPCR reactions on single sorted E. coli.

FIG. 30 is a graph of the mean C_{T} values from reactions depicted in FIG. 29 at each template dilution.

FIG. 31 is a graph depiciting qPCR curves for 16S rRNA qPCR of single sorted S. typhimurium and E. coli and multiple cells of each species.

FIG. 32 is a plot showing the mean CT values and standard deviation for single and multiple cell reactions in FIG. 31.

FIG. 33 is a plot of 16S rRNA copy number yielded PCR-based WGA reactions.

FIG. 34 is a plot 16S rRNA copy number yielded from microfluidic MDA reactions.

FIG. 35 is a graph of copy number of 10 loci yielded from microfluidic single-cell E. coli MDA reactions performed without DTT.

FIG. 36 is a graph of read coverage of the Delftiaacidovorans reference genome by sequencing data from two separate single E. coli microfluidic MDA reactions.

FIG. 37 is a graph of read coverage of the E. coli reference genome by sequencing data from (A) unamplified genomic DNA, (B) nanolitre MDA, (C) combined nanolitre/microlitre MDA, (D) microlitre MDA, (E) and nanolitre no-cell control MDA.

FIG. 38 is a graph of overlaid normalized read coverage of the E. coli reference genome by sequencing data from two separate single-E. coli nanolitre MDA reactions (red and cyan). Overlapping regions are in dark cyan.

FIG. 39 is a graph of reference genome coverage versus mean sequencing coverage depth for each identified sample.

FIG. 40 is a histogram showing coverage depth of reference genome for each MDA reaction type at a mean coverage depth of 16×. For each sequenced sample, the coefficient of variation (CV) of the coverage for each position of the reference genome is shown.

FIG. 41 is a summary of taxonomic profiles uncovered in metagenomes of 67 WGA samples originating from three distinct environments. A) Superimposed GC kernel density plot for all contigs generated from assemblies of individual metagenomic datasets. B) Hierarchical cluster analysis of sample-specific taxonomic profiles generated through a MEGAN analysis of blastx sequence comparisons against the RefSeq proteomic database. C) Taxonomic profiles of three environment-representative metagenomes, as generated through three distinct procedures (MLTreeMap, blastx against egg NOG, blastx against RefSeq proteomic).

FIG. 42 is a set of micrographs of primary tumour cell nuclei showing the morphological heterogeneity of the sample.

FIG. 43 are micrographs of a primary breast cancer pleural effusion cell nucleus (A) in the cell-sorting module and (B) in a stored droplet.

FIG. 44A is a graph depicting qPCR curves for RNase P qPCR of single sorted primary breast cancer pleural effusion cell nude.

FIG. 44B is a plot of the mean CT values and standard deviation for all reaction types indicated in FIG. 44A.

FIG. 45 is a graph depicting qPCR curves for 6-plex PCR of single sorted primary breast cancer pleural effusion cell nuclei.

FIG. 46 are capillary electrophoresis plots of PCR amplicons for 5 somatic mutation loci (left to right: FGA, GOLGA4, KIAA1468, KIF1C, and MORC1) from 4 on-chip single-nuclei multiplex PCR reactions.

FIG. 47 are histograms showing read coverage binned by chromosome for 5 loci amplicons from (A) purified genomic DNA and (B) on-chip multiplex PCR of a single primary breast cancer pleural effusion cell nucleus.

FIG. 48A is a graph showing GAPDH qRT-PCR of dilutions of purified RNA(A) qRT-PCR curves for all reactions listed. (B)

FIG. 48B is a plot of the mean CT values fitted to a line and standard deviation for all reactions in FIG. 48A.

FIG. 49 Quantification of GAPDH cDNA in WTA product by qPCR. (A) qPCR curves for all reactions, (B) Mean CT values fitted to a line and standard deviation for all reactions.

FIG. 50 Heat map depicting CT values for 48 qPCR assays applied to WTA product.

FIG. 51 Standard curves of mean CT values from selected qPCR assays applied to on-chip WTA products.

FIG. 52 Comparison of gene abundances relative to 18S rRNA in on-chip and in-tube WTA product. (A) Gene abundances in on-chip WTA product averaged over 2, 20, and 200 pg of input RNA quantities, (B) Gene abundances in in-tube WTA product from 100 ng input RNA.

### DETAILED DESCRIPTION

### Definitions

A “microfluidic device”, as used herein, refers to any device that allows for the precise control and manipulation of fluids that are geometrically constrained to structures in which at least one dimension (width, length, height) generally may be less than 1 mm.

“Flow” or “flowing”, as used herein, refers to moving fluid through a device or in a method of the invention and encompasses, without limitation, movement of a fluid, with or against the stream, whether or not the fluid is carried by the stream. For example, the movement of droplets through a device or in a method described herein, e.g. through channels of a storage element or microfluidic chip of the invention, comprises a flow. This is so, according to the invention, whether or not the droplets are carried by a stream of continuous phase fluid also comprising a flow, or whether the droplets are caused to move by some other direct or indirect force or motivation. The application of any force may be used to provide a flow, including without limitation, pressure, capillary action, electro-osmosis, electrophoresis, dielectrophoresis, optical tweezers, or any combination thereof.

The direction of fluid flow through a device as described herein dictates an “upstream” and a “downstream” orientation of the droplet storage element. Accordingly, an inlet will be located at an upstream position of the droplet storage element, and an outlet will be generally located at a downstream position of the droplet storage element.

“Continuous phase” or “continuous phase stream”, as used herein, refers to a fluidic stream that is flowed as a single contiguous entity. Flow of the continuous phase may be laminar, or turbulent in some cases. The continuous phase may, in places, enclose an interior space that is filled, or partially filled, with a second fluid (such as a dispersed phase droplet as defined below).

“Continuous phase fluid” or “carrier fluid”, as used herein, refers to the fluid forming the continuous phase. Any continuous phase fluid that does not absolutely prevent the wetting of stationary dispersed phase droplets to a given surface, and does not stabilize dispersed phase droplets such that they cannot be merged, may be suitable for the purposes of the invention.

“Dispersed phase” or “discontinuous phase”, as used herein, refers to any fluid stream that is not produced as a single entity. The dispersed phase may have the appearance of individual droplets, optionally surrounded by a second fluid, i.e. continuous phase fluid.

“Dispersed phase fluid” or “discontinuous phase fluid” as used herein, refers to the fluid forming the dispersed phase. The dispersed phase fluid can include a biological/chemical material. The biological/chemical material can be tissues, cells, particles, proteins, antibodies, amino acids, nucleotides, small molecules, pharmaceuticals, etc. The biological/chemical material can include one or more labels. The label can be a DNA tag, dyes, quantum dot, etc. or combinations thereof.

“Droplet” or “dispersed phase droplet” as used herein, refers to an isolated portion of a dispersed phase fluid that is at least partially surrounded by, or immersed within, a second fluid with which the droplet is immiscible, i.e. continuous phase fluid. For example, a droplet may be completely surrounded by continuous phase fluid or may be bounded by continuous phase fluid and one or more surfaces of a microfluidic device. If the continuous phase fluid is an oil, for example, droplets may be aqueous, or may be mixtures including aqueous and non-aqueous components. If the continuous phase fluid is aqueous, for example, droplets may be an oil, or may be mixtures including oil and non-oil components. Droplets may take a wide variety of shapes. In some cases, the droplets may be spherical or substantially spherical; however, in other cases, the droplets may have non-spherical shapes, depending on the circumstances, including, but not limited to generally disc shaped, slug shaped, truncated sphere, ellipsoid, partially compressed sphere, hemispherical, ovoid, cylindrical, and various shapes formed during procedures that are performed on the droplet, such as merging or splitting.

The terms “continuous phase” (or “carrier fluid”) and “dispersed phase” (or “discontinuous phase”) are relative terms which refer to the characteristics of the fluids during interactions when the continuous phase fluid is more prevalent than the dispersed phase fluid. As used herein, however, the continuous phase may still be considered the continuous phase even when the dispersed phase may be more prevalent e.g. when a dispersed phase droplet is being eluted from a storage element as described herein. Similarly, A dispersed phase, as used herein, may still be considered the dispersed phase in the absence of a continuous phase or where the continuous phase fluid is less prevalent, e.g. during elution of a droplet from a storage element as described herein.

An “emulsion”, as used herein, refers to a mixture of two or more fluids, wherein at least two of the two or more fluids are normally immiscible (un-blendable) at the given temperature and pressure. In an emulsion, small globules of a first (or more) fluids (the “dispersed phase”) are dispersed or suspended in a second fluid with which the first or more fluids will not mix (the “continuous phase”), but the first or more fluids of the dispersed phase may mix with each other.

An “inlet” or an “outlet”, as used herein, may include any aperture whereby fluid flow is restricted through the inlet, outlet or aperture. There may be a valve to control flow, or flow may be controlled by separating the channels with a layer which prevents flow (for example, oil).

“Contaminants”, as used herein, refers to any material that may interfere with the precision and/or accuracy of the assays of the cell or cell contents. Contaminants include, but are not limited to proteins, small molecules, salts, buffers, RNA, DNA, other cells, particles, and so forth.

“Wettability” of a surface, as used herein, refers to the degree to which a liquid is able to maintain contact with a solid surface, resulting from intermolecular interactions when the two are brought together. Wettability may be measured in terms of the contact angle between a droplet of the liquid in thermal equilibrium and a horizontal surface. A contact angle of 0 degrees corresponds to “perfect” wetting, and the droplet may spread to form a film on the surface. Wettability is a thermodynamic variable that depends on the interfacial tensions of the surfaces.

A “channel”, as used herein, refers to a generally tubular passage or conduit for fluids. According to various embodiments of the invention, a channel will have a surface that is in contact with one or more fluids.

“Rupture”, as used herein, refers to breaking or breaching the cohesive forces that maintain the surface of a fluid intact.

“Combine” or “combining” or “merging”, as used herein, refers to the coalescence of two or more discrete dispersed phase droplets, and their contents, into a single, unitary droplet.

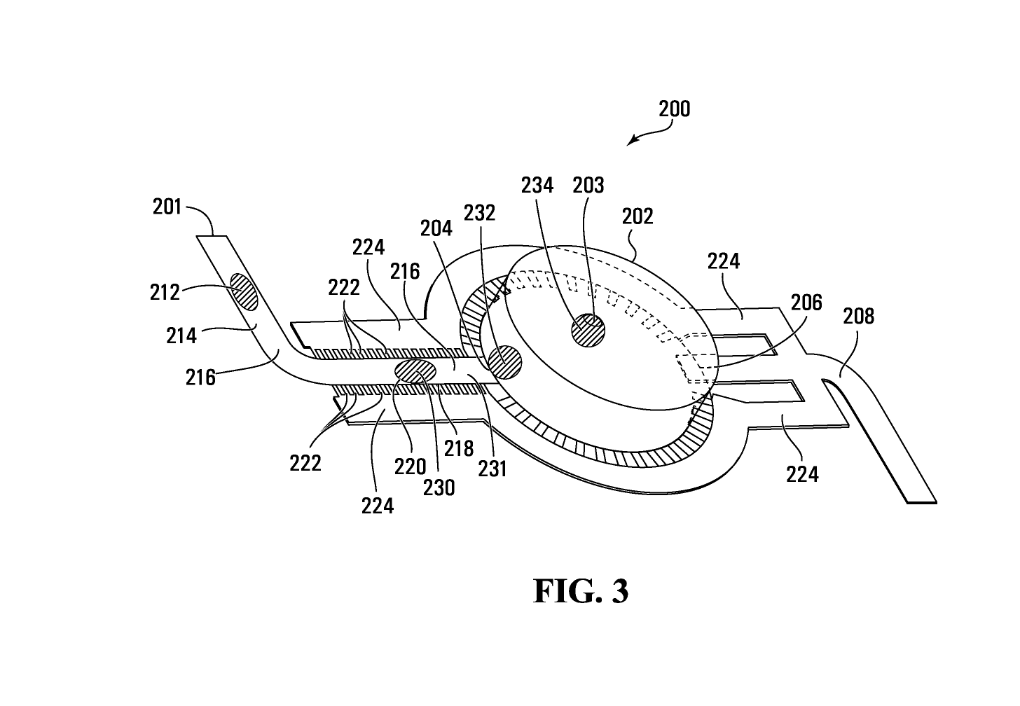

“Storage element”, as used herein, refers to a microfluidic structure operable to immobilize, and indefinitely retain, a dispersed phase droplet flowed therein. The storage element may be configured as in FIG. 3, wherein a droplet or droplets will remain in the storage element provided that the volume thereof does not exceed the volume of the retaining chamber.

A “dispersed phase retaining chamber” or “dispersed phase droplet retaining chamber” or “retaining chamber”, as used herein, refers to a structure that is operably configured to indefinitely retain one or more portions of dispersed phase fluid deposited within in it. According to various embodiments of the invention, a dispersed phase retaining chamber will be configured to retain any and all portions of dispersed phase fluid deposited within it, provided that the total volume of dispersed phase fluid deposited within the retaining chamber does not exceed the volume of the retaining chamber. A “portion” of the dispersed phase, as used herein, may refer to a discrete droplet or a plurality of discrete droplets.

An “elution stream”, as used herein, refers to a dispersed phase droplet that exits, has exited, or is operable to exit a storage element as described herein. According to various embodiments of the invention, portions of dispersed phase fluid retained within a retaining chamber become an elution stream when the total volume of dispersed phase fluid within the retaining chamber exceeds the volume of the retaining chamber.

A “sieve element”, as used herein, refers to a small side channel branching off a main channel through which a droplet is flowed. Sieve elements have a diameter smaller than a droplet and serve to divert continuous phase fluid from the main channel.

“Critical velocity”, as used herein, refers to the velocity of a dispersed phase droplet at or below which a film of continuous phase fluid separating the dispersed phase droplet from a surface will rupture.

“Critical incoming droplet velocity”, as used herein, refers to the velocity of a dispersed phase droplet as it enters a storage element which will result in the droplet wetting the surface of the storage element at the inlet to the retaining chamber.

### Droplet Wetting

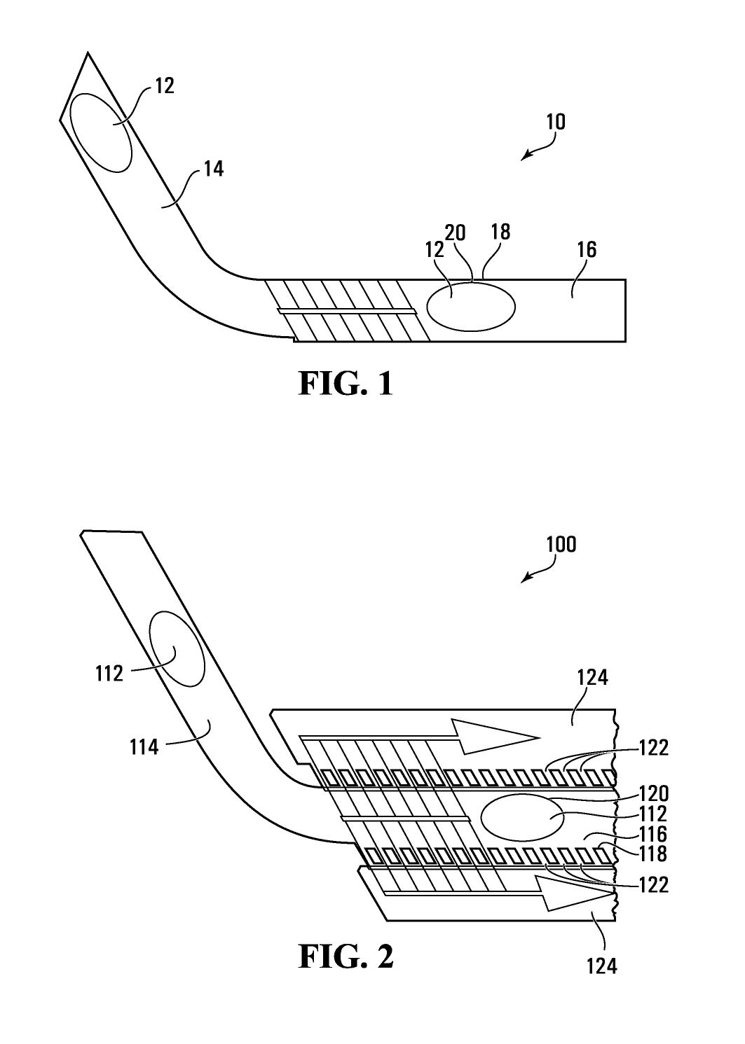

Referring to FIG. 1, an apparatus for use in a flow-controlled method of determining the position at which a dispersed phase droplet wets a surface of a channel according to a first embodiment of the invention is shown generally at 10. A flow-controlled wetting method enables precision positioning, wetting, and merging of arbitrary sequences of dispersed phase droplets on the surface of a channel at of an addressable storage element. This method exploits various fluid physics phenomena: the formation of a thin film of viscous continuous phase fluid around a dispersed phase droplet flowing through a channel, the rupture of this film when its thickness is sufficiently reduced, and contact line pinning. The advance of a liquid-liquid boundary at a non-ideal solid interface (capillary surface) exhibits significant hysteresis (23), leading to a retentive force that resists droplet motion under flow (24). Using this force for droplet immobilization creates a conflict between requirements, since the transport of droplets without fouling or cross-contamination requires that they not contact the channel surface (25). Although these requirements may be satisfied by chemically modifying surface properties at desired locations (26), this can be difficult to implement. Instead, the hydrodynamic flow may be used to prevent droplets from contacting channel walls during flow while preserving the ability to wet channel walls at defined storage regions, without modification of the device surface properties.

Referring again to FIG. 1, a dispersed phase droplet 12 immersed within a continuous phase fluid 14 is flowed down a channel 16 having a channel surface 18. Droplet 12 is separated from the channel surface 18 by a thin lubricating film 20 of continuous phase fluid 14 (27) with a thickness that is a function of the velocity of the droplet. If the droplet velocity, and hence the film thickness, is reduced to a critical value, an instability arises in which intermolecular forces between the droplet 12 and the surface 18 cause the film 20 to spontaneously rupture, allowing the droplet to wet the surface of the channel (28). The critical film thickness for spontaneous rupture of the film 20 surrounding the droplet 12 is given by equation 1:

$${h}_{0} = {( \frac{{AR}^{2}}{{ξ}_{max} γ} )}^{\frac{1}{4}}$$

where h₀ is the critical film thickness, A is the Hamaker constant taken to be 10⁻²⁶ J between PDMS and water (29), R is the radius of the approximated disc of carrier fluid separating the droplet from the channel wall, ξₘₐₓ is a numerical constant associated with the most unstable mode of a perturbation to the uniform film (30), and γ is the interfacial tension at the continuous phase/disperse phase interface. The smallest value of ξₘₐₓ is used as it leads to the greatest instability of the film. The value of h₀ can then be substituted into equation 2 below to find the “critical velocity” (U) at which the film thickness is reduced to the critical thickness and spontaneous wetting of the droplet to channel surfaces occurs:

$$b = 0.643 {r ( \frac{3 μ U}{γ} )}^{\frac{2}{3}}$$

where b is the film thickness, r is half the height of the droplet, and μ is the viscosity of the continuous phase. Selective wetting may therefore be achieved without modification of surface properties, i.e. where the channel surface 18 has a uniform wettability, by engineering the device geometry and controlling flow to maintain the droplet velocity above this critical value until arrival at the desired position in the channel 16.

Referring to FIG. 2, a microfluidic device for use in a flow-controlled method of determining the position at which a dispersed phase droplet wets a surface of a channel according to an embodiment of the invention is shown generally at 100. A dispersed phase droplet 112 immersed within a continuous phase fluid 114 is flowed down a main channel 116 having a channel surface 118. Droplet 112 is separated from the channel surface 118 by a thin lubricating film 120 of continuous phase fluid 114 with a thickness that is a function of the velocity of the droplet. In this embodiment, the velocity of an incoming droplet is reduced as the droplet enters main channel 116 by diverting a portion of the flowing continuous phase fluid 114 from the main channel through a series of sieve elements 122 reminiscent of Niu et al. (31) to bypass channels 124. The high interfacial energy required to deform droplet 112 ensures that it does not pass through sieve elements 122. Bypass channels 124 serve to keep diverted continuous phase fluid out of main channel 116 at least until the droplet velocity, and hence the film thickness, is reduced to a critical value at which the film 120 spontaneously ruptures, allowing the droplet to wet the surface of the main channel. Accordingly, bypass channels 124 may serve to permanently divert a portion of continuous phase fluid from main channel 116, or merely divert the portion to a position within main channel that is downstream from the position at which the droplet 112 has wet the channel surface 118. In FIG. 2, sieve elements 122 are oriented generally perpendicular to the direction of flow through main channel 116, however, a person skilled in the art will understand that the sieve elements could be oriented in a variety of ways that will effectively divert continuous phase fluid from the main channel.

Referring to FIG. 3, a storage element for use in a flow-controlled method of immobilizing and retaining a dispersed phase droplet for processing according to a third embodiment of the invention is shown generally at 200. Storage element 200 includes a dispersed phase retaining chamber 202 that is in fluid communication, via retaining chamber inlet 204, with a main channel 216 having a channel surface 218. In this embodiment, the dispersed phase retaining chamber is cylindrical, however, a person skilled in the art will understand that the retaining chamber can have a variety of shapes. A series of sieve elements 222 branch off from main channel 216 to connect the main channel with bypass channels 224. Bypass channels 224 are further in fluid communication retaining chamber 202. Retaining chamber 202 is further in fluid communication with retaining chamber outlet 206. Retaining chamber outlet 206 merges with bypass channels 224 to form elution outlet 208.

In operation, a first dispersed phase droplet 212 immersed within a continuous phase fluid 214 is flowed into storage element 200 via storage feed channel 201 and down main channel 216. Droplet 212 is separated from the channel surface 218 by a thin lubricating film 220 of continuous phase fluid 214 with a thickness that is a function of the velocity of the droplet. In this embodiment, the velocity of an incoming droplet is reduced as the droplet enters main channel 216 by diverting a portion of the flowing continuous phase fluid 214 from the main channel through sieve elements 222 into bypass channels 224. The high interfacial energy required to deform droplet 212 ensures that it does not pass through sieve elements 222. Bypass channels 224 serve to keep diverted continuous phase fluid out of main channel 216 at least until the droplet velocity, and hence the film thickness, is reduced to a critical value at which film 220 spontaneously ruptures, allowing the droplet to wet the surface of the main channel. Accordingly, bypass channels 224 may serve to divert a portion of continuous phase fluid from main channel 216. In FIG. 3, sieve elements 222 are oriented generally perpendicular to the direction of flow through main channel 216. However, a person skilled in the art will understand that the sieve elements could be oriented in a variety of ways that will effectively divert continuous phase fluid from the main channel.

“Critical incoming droplet velocity”, as used herein, refers to the velocity of a dispersed phase droplet as it enters a storage element which will result in the droplet wetting the surface of the storage element at the retaining chamber inlet. When a dispersed phase droplet 212 enters the storage element 200 with a velocity less than or equal to the critical incoming droplet velocity, it wets the surface 218 of main channel 216 at a first position 230 upstream of the retaining chamber 202. When dispersed phase droplet 212 enters the storage element 200 at the critical incoming droplet velocity, the flow velocity at the retaining chamber inlet 204 will be less than or equal to the velocity, and the droplet will wet at a second position 231 at or adjacent to the chamber inlet. Once the leading edge of dispersed phase droplet 212 enters the retaining chamber 202 via retaining chamber inlet 204, it is pulled in by surface tension where it wets the retaining chamber's sidewall, precisely positioning it at third position 232 adjacent the chamber inlet. If the flow velocity of dispersed phase droplet 212 at retaining chamber inlet 204 exceeds the critical incoming droplet velocity, the droplet is not sufficiently decelerated by diversion of continuous phase fluid 214 by the sieve elements, and the droplet will not wet in the main channel 216 or at the retaining chamber inlet, but will travel into retaining chamber 202 and follow an upward trajectory to wet at a fourth position 234 on chamber ceiling 203.

While the fourth position 234 in FIG. 3 is depicted as being substantially at the center of chamber ceiling 203, the person skilled in the art will understand that the position at which droplet 212 wets to the ceiling will be dictated by the velocity with which the droplet enters retaining chamber 202, laminar flow, and droplet buoyancy. Moreover, the position may be located virtually anywhere on the ceiling or retaining chamber walls, and not necessarily co-linear with main channel 216. Furthermore, the positioning of droplets may be further influenced by changing the surface properties of the storage element at various positions.

Once docked inside the retaining chamber 202, the droplet 212 is immobilized indefinitely and sequestered from high flow of continuous phase fluid 214 such that the droplet would not be dislodged by flowing continuous phase fluid through the storage element 200 at a maximum velocity from the storage element inlet 201. Regardless, the high interfacial energy required to deform droplet 212 further ensures that it does not pass through retaining chamber outlet 206.

### Droplet Merging

According to further embodiments of the invention, additional dispersed phase droplets may be subsequently flowed into storage element 200 once a first dispersed phase droplet 212 has been immobilized. The precise position at which each subsequent droplet wets the surface of the storage element, whether within main channel 216 or retaining chamber 202, depends on the velocity of the subsequent droplet as it flows through the storage element. If the flow rate is kept constant, subsequent droplets will be delivered to, and wetted to the surface of the storage element at, the same position as the first stored droplet, and held in contact with the stored droplet indefinitely, thereby allowing sufficient time for coalescence even when partially stabilizing surfactants are used. In the absence of a surfactant in the disperse phase fluid, droplets coalesce shortly after making contact, thereby allowing for the sequential merging of droplets at maximal flow rates.

Few surfactants have been shown to be capable of stabilizing aqueous droplets in fluorocarbon oils (32; 33). Interfacial phenomena are, however, important during the formation of droplets. In the absence of a fluorosurfactant in the continuous phase, droplets may wet the channel walls during injection, leading to the formation of satellite droplets and introducing potential for cross-contamination. The inclusion of a fluorosurfactant (17% PFO) in the continuous phase (25) was found to suppress wetting during droplet injection for all aqueous reagents tested. This surfactant does not prevent the wetting of stationary droplets to untreated PDMS channels and does not stabilize droplets (32).

In addition to surfactants in the continuous phase, a second dispersed phase surfactant may be included to reduce the adsorption of analytes to channel walls or droplet surfaces. The inclusion of this dispersed phase surfactant may partially stabilize droplets, and significantly increase the time required for coalescence. Thus, when using aqueous surfactants, the robust merging of droplets may require that they be held in contact for an extended time. Nevertheless, the present devices and methods allow for such control. Regardless, once the total volume of droplets sent to a storage element is large enough to occupy a significant fraction of the chamber volume (˜25%), all droplets merge with the stored droplet. Thus, if the final stored droplet volume is sufficiently large and the sequence of droplet merging is unimportant, flow velocities much higher than the critical incoming droplet velocity can be used to achieve faster formulation.

Alternatively, a plurality of dispersed phase droplets may be stored at discrete positions within a single storage element. Again, the precise position at which each subsequent droplet wets the surface of the storage element, whether within main channel 216 or retaining chamber 202, depends on the velocity of the subsequent droplet as it flows through the storage element. Accordingly, the flow rate can be adjusted to deliver a second dispersed phase droplet to a second position within the storage element, at which second position the film of continuous phase fluid separating the second droplet from the surface of the storage element ruptures and the second droplet wets the surface.

Similarly, the flow rate can be adjusted to deliver a third dispersed phase droplet to a third position within the storage element, at which third position the film of continuous phase fluid separating the third droplet from the surface of the storage element ruptures and the second droplet wets the surface. If the third position is sufficiently close to the first position such that the third dispersed phase droplet contacts the first dispersed phase droplet, the first dispersed phase droplet and third dispersed phase droplet may be held in contact indefinitely, thereby allowing sufficient time for merging. Alternatively, if the third position is sufficiently close to the second position such that the third dispersed phase droplet contacts the second dispersed phase droplet, the second dispersed phase droplet and third dispersed phase droplet may be held in contact indefinitely, thereby allowing sufficient time for the second dispersed phase droplet and the third dispersed phase droplet to combine. In yet a further alternative, if the third position bridges the first position and the second position, such that the third droplet contacts the first dispersed phase droplet and the second dispersed phase droplet, the third dispersed phase droplet may be held in contact with the first dispersed phase droplet and the second dispersed phase droplet indefinitely, thereby allowing sufficient time for all three dispersed phase droplets to combine into a single stored droplet.

As the person skilled in the art will understand, this droplet storage process allows for the formulation of complex mixtures by sequential or non-sequential combination of different droplet types directly in the storage element, including droplets of different sizes and chemical content For example, droplets comprising different substrates, catalysts (including enzymes), reagents, buffers, etc. can be delivered to and combined within the storage element, sequentially or non-sequentially. This enables precise formulation and storage of multi-step reactions. The addressability of the storage element array and the properties of two-phase flow further allow for selective elution of any stored droplet without disturbing neighboring chambers.

When operating at or below the critical velocity, 100% coalescence may be routine. Again, if the final stored droplet volume is sufficiently large and the sequence of droplet merging is unimportant, flow velocities much higher than the critical incoming droplet velocity can be used to achieve faster formulation.

### Droplet Elution

The volume of retaining chamber 202 defines an upper limit on the volume of a stored droplet, or droplets, that it can contain, above which overfilling of the chamber occurs where droplets are pinched off and leak out of the storage element. In other words, retaining chamber 202 is operably configured to retain stored droplet 212 provided that the volume of the stored droplet is less than the volume of the retaining chamber.

Thus, according to another embodiment of the invention, a stored droplet stored within retaining chamber 202 may be eluted from storage element 200 by flowing an elution droplet of dispersed phase fluid into the retaining chamber, wherein the combined volume of the stored droplet and the elution droplet exceeds the retaining chamber volume. The incoming elution droplet coalesces with the stored droplet to form an elution stream, which begins to exit retaining chamber 202 through retaining chamber outlet 206 when volume of the elution stream within the retaining chamber is exceeded. This elution method ensures that the contents of eluted droplets are always encapsulated in the continuous phase fluid and do not come in contact with the storage element walls subsequent to exiting retaining chamber 202. In practical terms, the contents of a stored droplet may be completely eluted with a volume of elution stream that is 12.5 times the volume of retaining chamber 202.

### Arrays

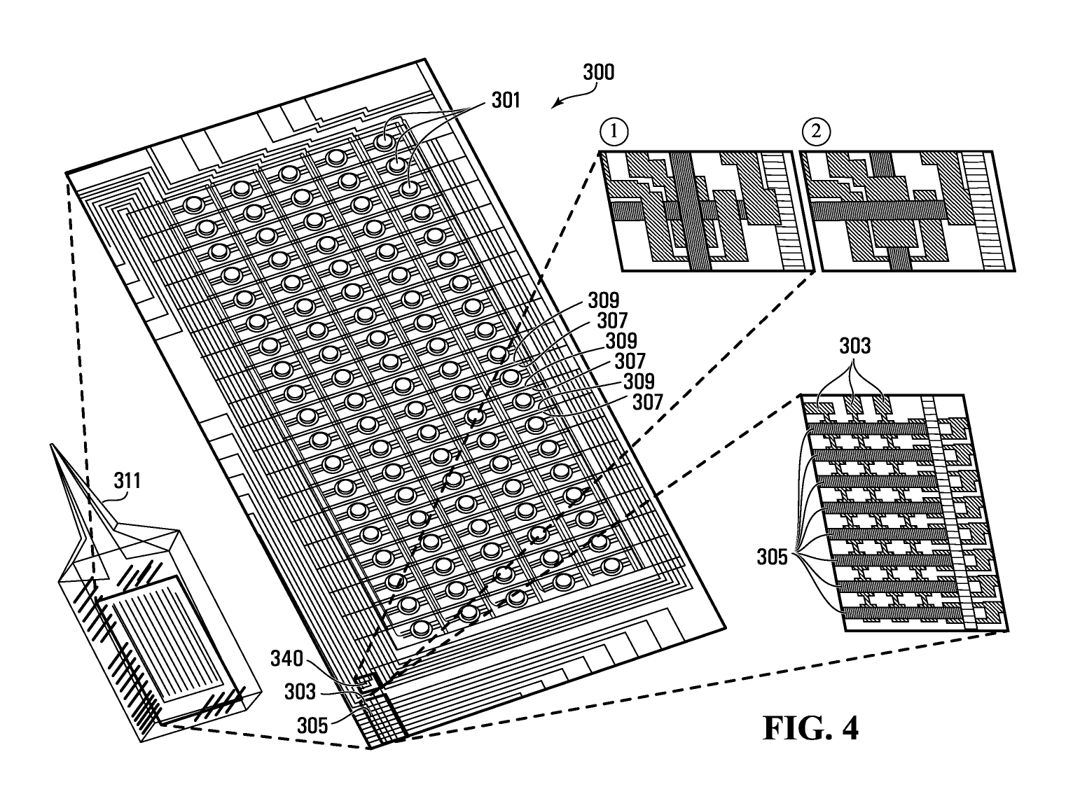

Referring to FIG. 4, a microfluidic device comprising an addressable array of storage elements according to another embodiment of the invention is shown generally at 300. Device 300 is comprised of a two-dimensional addressable array of 95 storage elements 301 as previously depicted in FIG. 3, organized into 19 rows and 5 columns. Reagents may be individually delivered to, and reaction products may be individually extracted from, each storage element 301. In operation, device 300 is flooded with continuous phase fluid and all reagents are handled in the form of dispersed phased droplets. Three-valve peristaltic pumping 303 is used to dispense arbitrary volumes of reagents from 8 reagent inlets 305 where each pump cycle advances a discrete volume of dispersed phase droplet. User-defined volumes can thus be metered out in discrete increments using a programmed number of pump cycles (34, 35). The volume metered using a single pump cycle is referred to herein as a “pump increment”. Reagents may be pumped into a flowing stream of continuous phase fluid, and the dispensed volume may then be broken off into a dispersed phase droplet by actuation of a valve.

Each storage element 301 is individually addressed by using a multiplexer known in the art, e.g. (36), to select the active row and a series of column valves to select the active column. Valve actuation patterns create a unique fluidic path that passes from the high-pressure continuous phase fluid input, past the droplet metering unit to the selected storage element, and out to one of two low pressure outlets (waste or elution). Dispensed dispersed phase droplets are transported by continuous phase fluid flow to the addressed storage element, which merges all incoming droplets into a stored droplet, formulating the desired solution. Dispersed phase droplets are delivered to storage elements 301 of each row via a common feed channel 307, and elution streams are collected from the storage elements of each row by a common elution channel 309. During elution of storage elements 301, the contents of the selected storage element are flushed to the elution nozzle 311 which enables dispensing directly into standard microfuge tubes.

To enable automated droplet recovery, device 300 may be mounted to a custom 3-axis robotic setup (for example, see FIG. 14) controlled by software that coordinates stage motion with droplet elution. Deposition of eluted droplets from each chamber may be achieved using a zero dead-volume elution nozzle 311 that is designed to fit into standard microfuge tubes or microwell plates. Between droplet elutions, the nozzle may be rinsed in isopropanol to wash away any satellite droplets that may remain attached to the nozzle's exterior that can lead to sample carry-over.

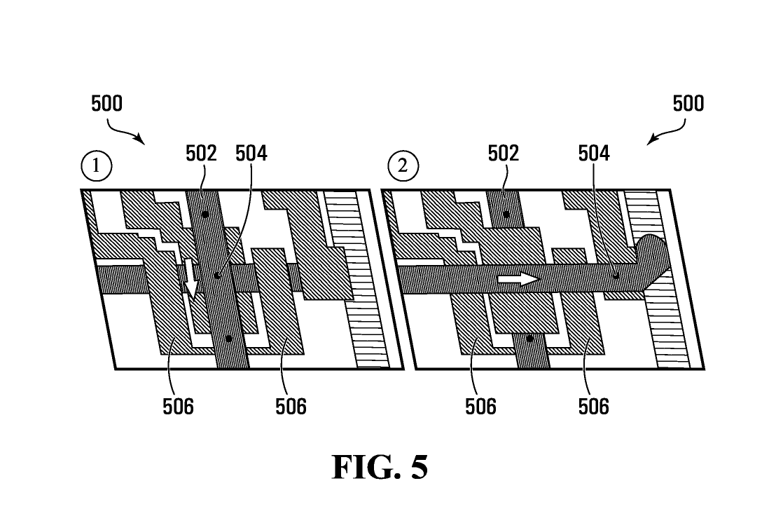

Device 300 may further include integrated cell-sorting module 340 for selecting intact cells for delivery to the array. Referring to FIG. 5, a cell-sorting module is shown generally at 500. Module 500 allows for visual sorting of single cells from suspension into dispersed phase droplets, which can then be delivered to any storage element and combined with reagent droplets for analysis. A suspension of single cells suspension may be pumped down a sorting channel 502 while the isolation area is visually monitored by microscopy. When a cell of interest 504 is identified, valves 506 are actuated to isolate it, and the cell is pumped into a droplet for delivery to the storage array. The strategy used is similar to that used by Marcy et al. (9), but combined with the droplet-based functionality of this device, allows for more versatility in single cell handling.

This microfluidic platform combines the advantages of droplets with microvalve technology to enable precise formulation and storage of multi-step reactions. The addressability of the storage element array and the properties of two-phase flow further allow for selective elution of any stored droplet without disturbing neighboring chambers.

A person skilled in the art will understand that the device architecture described in FIG. 4 may be scaled considerably with only modest increases in control complexity. For example, increasing the number of control lines in the factorial multiplexer from 6 to 10 would allow for the addressing of 252 rows, corresponding to an array of 1260 storage elements using a 5 column-layout. Further improvements may be readily achieved by reducing channel resistances to increase droplet transport velocity, and by reducing the area of each storage element.

A person skilled in the art will further understand that the methods and systems of wetting and immobilizing droplets as disclosed herein could complemented with additional features affected wetting. Surface features could be introduced to the storage element which facilitate wetting. Surface energy patterning in the main channel or retaining chamber can be modified to reduce the critical thickness of the film continuous phase fluid separating a droplet from the surface, for example, by photografting patterned hydrophillic poly(acrylic acid) to the surface (42). Alternatively, if surfactants are utilized, electro-wetting may be additional employed to assist in wetting and droplet mergin.

### Stored Droplet Operations

The combined capabilities of high precision formulation, programmability, and automated elution allow for the execution of long and multistep protocols without user intervention, making this system well-suited to applications where careful optimization of protocols and reaction conditions is needed and where samples are limiting. Examples of such formulation problems include enzyme characterization, protein crystallization, the optimization of molecular biology protocols, and combinatorial chemical synthesis.

Single devices according to various embodiments of the invention are amenable to use for a variety of different analyses. For example, a variety of single cell experiments, all requiring different liquid handling protocols, may be conducted using the same device. These include phenotypic sorting, culturing of single bacteria from a mixed suspension, PCR-based species identification of single bacteria through recovery and sequencing of single cell PCR amplicon, and multi-step Whole Genome Analysis on single cells sorted from a biofilm. The devices described herein are equally applicable to microbes and larger eukaryotic cells and, coupled with the demonstrated sensitive and efficient amplification of nucleic acids, which makes these systems attractive for single cell genomic analysis with applications in reproductive medicine and cancer research. The devices may also be used to place multiple selected single cells in the same small volume to allow for interrogation of predator-prey or pathogen-host interaction at the single cell level.

### Example 1

### 1.1 Device Architecture and Operation

Microfluidic devices as depicted in FIG. 4 were fabricated for use in several microfluidic applications. A droplet-metering unit comprising three-valve peristaltic pumping (4) was used to dispense arbitrary volumes of reagents from 8 aqueous inlets with a pump increment of approximately 133 pL. User-defined volumes of dispersed phase droplets can thus be metered out in discrete incremental portions using a programmed number of pump cycles. The device further includes an integrated cell sorter as depicted in FIG. 5. The storage element design included a main channel 520 μm in length and 10 μm in height, with 18 sieve elements (30 μm×10 μm×5 μm) along each side of the main channel,

### 1.2 Microfluidic Device Fabrication and Operation

Devices described herein were fabricated using multilayer soft lithography. Generally, the devices have a three-layer design: The top layer was a “flow layer,” containing channels for droplet manipulation. The middle layer was a “control layer,” containing channels used for pneumatic valves. The bottom layer was a “blank layer,” to which control channels were sealed. All devices were made from polydimethylsiloxane (RTV615; General Electric). Devices were bonded to glass slides after plasma treatment of the bottom of the device and the slide (Harrick Plasma). Photolithography masks were designed by using AutoCAD software (Autodesk) and used to generate high-resolution (20,000 dpi) transparency masks (CAD/Art Services). Molds were fabricated by photolithography on 10.2 cm silicon wafers (Silicon Quest International). The flow layer consisted of three different profiles: 5 μm-high rectangular frits, 12 μm-high rounded channels, and 180 μm-high cylindrical storage chambers. The 5 μm layer was made with SU8-5 negative photoresist (Microchem Corp.), the 12 μm rounded layer was made with SPR220-7 positive photoresist (Microchem Corp.), and the 180 μm layer was made with SU8-100 negative photoresist (Microchem Corp.). The control layer consisted of two different profiles: 25 μm-high rectangular channels used for valves and 5 μm-high features used for sections of control lines passing under flow channels where valving was unwanted. The 5 μm layer was made with SU8-5 negative photoresist and the 25 μm layer was made with SU8-2025 negative photoresist (Microchem Corp.). Resist processing was performed according to the manufacturer's specifications.

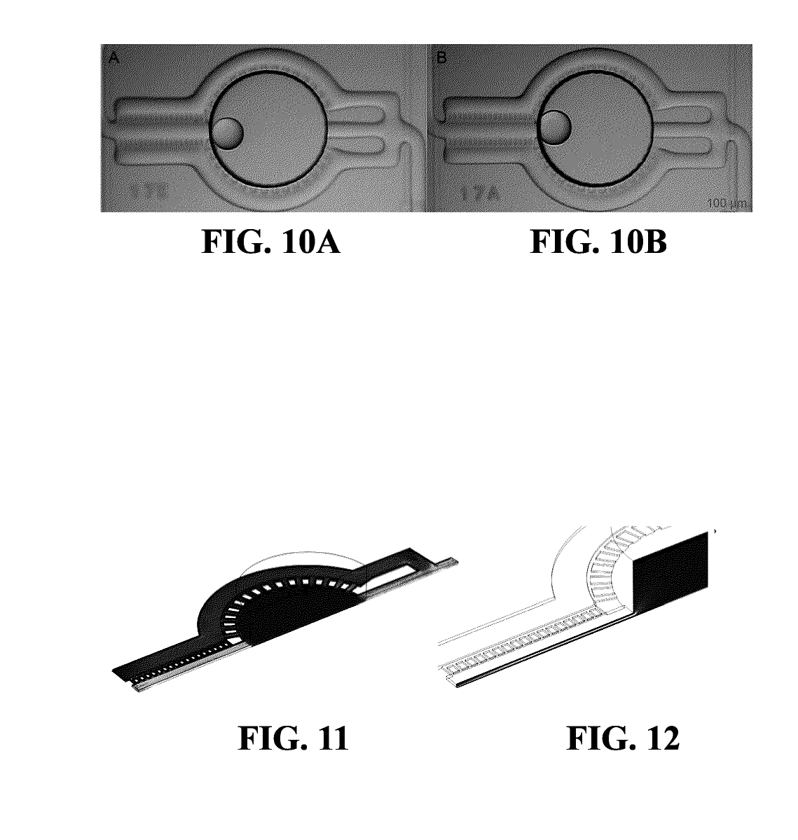

Microfluidic device operation was automated using custom software written in LabVIEW™ (National Instruments™). On-chip valve actuation was controlled using pneumatic solenoid actuators (Fluidigm™) connected to a PCI-6533 digital input/output card (National Instruments™). A single LabVIEW™ program was used to execute user-designed formulation scripts inputted as text files. Compressed air (5 psi-20 psi) was used to push reagents into the device. Prior to experiments, devices were dead-end filled with carrier fluid which was then flowed through the chamber array at 0.5 μL/min for ˜1 hr. Prior to elution of on-chip reactions into microfuge tubes, each tube is filled with light mineral oil, which wets PDMS preferentially over both the fluorinated carrier fluid and the aqueous phase, preventing any aqueous sample from adhering to the nozzle surfaces. The low density of light mineral oil also ensures that the eluted sample sinks to the bottom of the well, away from the nozzle tip. Between elution of each storage element, the nozzle is rinsed in isopropanol to wash away any aqueous droplets that may remain attached to the nozzle's exterior that can lead to sample carry-over. After elution into microfuge tubes is complete, additional water or buffer is added to each tube in order to provide sufficient volume for handling by pipette, and the tubes are spun down to ensure all of the aqueous phase coalesces at the bottom of the tube. Sample can then be extracted for further processing by pipetting from the bottom of the tube. Automated droplet elution was performed by mounting the device on a 3-axis robot built from three interconnected precision stages T-LSMO25A, T-LSR300D, T-LSR160D (Zaber™). The device is vacuum-sealed to the lowering arm using a vacuum pump (FIG. 10). LabView™ control was used to coordinate the stage position and device operation to automate insertion of the elution nozzle of the device into selected microfuge tubes during elution.

### 1.3 Reagents

During stored droplet formulation, a 5:1 mixture (v/v) of FC-40 (viscosity 3.4 cP) (Sigma Aldrich™) and 1H,1H,2H,2H-perfluorooctanol (PFO) (Sigma Aldrich™) was used as the continuous phase fluid. During elution, this was exchanged for a lower viscosity mixture of 5:1 (v/v) FC-72 (viscosity 0.64 cP) (Sigma Aldrich™) and PFO in order to achieve higher flow velocities for faster elution. Quasar 670 fluorescent dye was obtained from Biosearch Technologies™. PCR reactions on human gDNA template (Biochain™) were performed using the RNAse P FAM™ detection kit (Biorad) and Universal Fast PCR Mix™ (Biorad™), which includes a passive ROX fluorescent dye. On-chip PCR reactions amplifying a fragment of the 16S rRNA gene specific to K12 Escherichia coli were performed with primer sequences from Lee et al. (37) (500 nM each), LC green intercalating dye (Idaho Technology™ Inc.), and Itaq Supermix™ (Biorad™), which includes a passive ROX fluorescent dye. PCR reactions amplifying a fragment of the 16S rRNA gene in E. coli and Salmonella bacteria were performed as above but with the following primers: 5′-TCGTGTTGTGAAATGTTGGGTT-3′, 5′-TAAGGGCCATGATGACTTGAC-3′. All off-chip PCR reactions on bacterial DNA were performed using the same primers and primer concentrations as on-chip and iQ SYBR Green Supermix™ (Biorad™). All whole genome amplification (WGA) reactions were performed using the Picople WGA Kit for Single Cells™ (Rubicon Genomics™). For all on-chip PCR and WGA experiments, all aqueous solutions were supplemented with 0.1% Tween 20 surfactant to avoid reagent adsorption onto PDMS channel walls and droplet interfaces, and reagent proportions were used as recommended by the manufacturer.

Polymerase Chain Reaction (PCR).

On-chip qPCR was performed using a prototype version of the Biomark microfluidic qPCR instrument (Fluidigm™), consisting of a flatbed thermocycler equipped with a camera, fluorescent illumination, and filters. Off-chip qPCR was performed using a Chromo 4 thermocycler (Biorad™) and data was analyzed using Opticon Monitor™ 3 software (Biorad™). The thermocycling protocol for RNAse P PCR consisted of an initial hotstart at 95 C. for 20 s, followed by 40 cycles of 95 C. for 1 s and 60 C. for 30 s. To determine cross-contamination during device elution, consecutively eluted on-chip storage elements were diluted into 20 μL of water, and 2 μL of this was used as template in an off-chip PCR reaction. The thermocycling protocol for PCR reactions on all bacteria consisted of an initial hotstart at 95 C. for 3 min. which was also used to lyse cells, followed by 40 cycles of 95 C. for 10 s, 60 C. for 30 s, and 72 C. for 30 s.

Image Acquisition and Analysis.

Microfluidic devices were mounted onto a DMIRE2™ fluorescent microscope (Leica™) or a SMZ1500™ stereoscope (Nikon™) for imaging. Leica L5 and TX2 filter cubes were used to image GFP and RFP fluorescence respectively. Still images of the device were acquired using CCD cameras (Q imaging Retiga™ 4000R and Canon™ 50D). Videos were made using an IV-CCAM2 CCD camera (Industrial Vision Source™). A confocal scanner (Wellscope™, Biomedical Photometrics™) was used to acquire confocal fluorescent scans of the device.

To measure mean fluorescence intensity of stored droplets containing formulated fluorescent dye concentrations, the fluorescent confocal scan of the droplet array was manually analyzed using ImageJ™ software. Linear fitting of the data was performed using the curve fitting toolbox in MATLAB™.

All custom image analysis software described below was written in MATLAB™ (Mathworks™) and used functions from the Image Processing Toolbox™ Volumes of stored droplets in storage elements were computed assuming a spherical droplet geometry and using custom software to segment and determine the radius of stored droplets from microscopy images.

Custom software was written to analyze all on-chip qPCR images. For each cycle, droplets were first segmented using the passive ROX dye images. This dye was included in the PCR reaction mix for all on-chip reactions. Segmentation after each cycle is necessary since the high temperatures that the chip is heated to during PCR cause the positions of the droplets to shift slightly in the storage elements from cycle to cycle. A pixelwise division of the FAM probe or LC green intercalating dye image by the passive ROX dye image was used to normalize data for variations in illumination across the droplet array and to account for increase in signal due to evaporation. For each droplet, an amplification curve was generated by subtracting the median normalized pixel intensity for each cycle was from that of the first cycle, and removing linear components extracted from the pre-exponential phase. Manual thresholding of the amplification curves in the exponential phase was performed to determine the C_{T} of each droplet. For the RNAse P qPCR experiment, any reactions with a C_{T} greater than 2 standard deviations above the mean C_{T} corresponding to a single molecule were determined to be nonspecific amplifications and were classified as not detected.

Custom software was written to analyze fluorescent images acquired from on-chip culture of GFP and RFP-expressing bacteria. As the culture media used was slightly fluorescent in the GFP channel, the first GFP image was used to segment each droplet. The boundary of each droplet was then slightly dilated to generate a new boundary, which was used to identify droplets in all subsequent images. Since the incubation of the chip was performed at a relatively low temperature (25° C.), the droplets did not shift position significantly during the time interval between image acquisitions and this method was sufficient to identify all droplets for all images. To generate a growth curve for each stored droplet, fluorescence intensity was first integrated over each droplet for each image in both GFP and RFP channels and a moving average filter with a window width of 3 was applied to all datapoints between the 3^{rd} and final images for each droplet in order to remove noise. When comparing endpoint GFP and RFP fluorescence in each two-strain co-culture, normalization was performed by dividing the integrated fluorescence intensity in each channel from the final image by the culture with the highest endpoint integrated fluorescence intensity of each group of co-cultures seeded with the same number of cells.

Finite Element Simulation.

Simulation of fluid flow through the droplet storage element was performed using COMSOL v4.0a (COMSOL). Fluid properties of FC-40 were used.

### 1.4 Reagent Metering

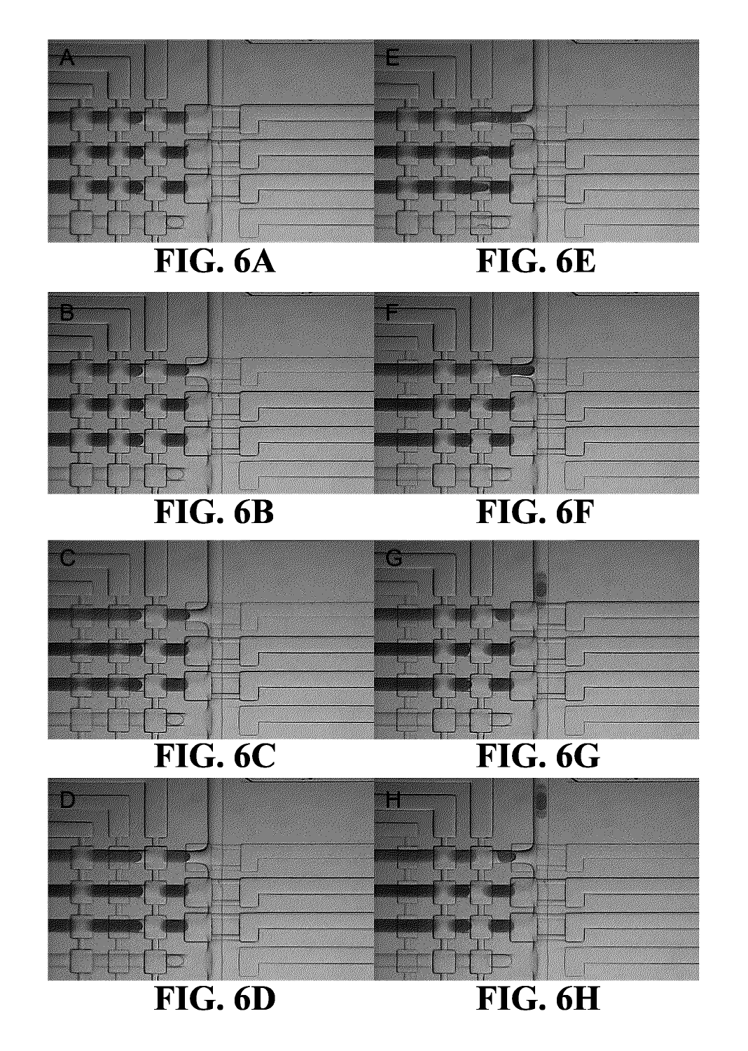

Programmable reagent dispensing, using a three-valve peristaltic pump was used to deliver arbitrary volumes of reagents in discrete increments from eight separate reagent inlets by varying the number of pump cycles (FIG. 3). Each pump increment was determined by the volume displaced by the middle valve of the pump 303. Devices were fabricated with pump increments of ˜133 pL or 150 pL. Reagent droplets were dispensed directly into a flowing pressure-driven stream of the carrier fluid, where they broke off through the combined effect of surface tension, shear stress, and valve actuation. All reagent inlet channels were designed to have the same length to prevent differences in fluidic resistance from affecting the metering precision of different reagents. Time course images acquired from a video of the droplet dispensing process are shown in FIG. 6. The cross-section of the channel into which droplets were dispensed has been designed to have a low aspect ratio and a sufficiently small area such that a single pump increment forms a droplet that occupies most of the channel's cross-sectional area. The droplet thus has an axial length longer than its cross-sectional diameter and is separated from the channel walls by a thin film of carrier fluid while in transit. The “pancaked” droplet is thus also in an energetically unfavourable state as its surface area is not minimized.

### 1.5 Droplet Docking and Merging by Flow-Controlled Wetting.

### Flow Controlled Wetting

A 5:1 mixture (v/v) of FC-40 and PFO was used as the continuous phase fluid. For the purposes of solving for h₀ using equation 1, R (the radius of the approximated disc of carrier fluid separating the droplet from the channel wall) is assumed to be 50 μm, and γ is 14 mJ/m² for a continuous phase fluid of FC-40+17% PFO. The smallest value of ξₘₐₓ, approximately 1.7, is used as it leads to the greatest instability of the film. For these values, h₀ is found to be approximately 8 nm. Substituting the value of h₀ into equation 2 to find the droplet velocity at which the film thickness is reduced to the critical thickness and spontaneous wetting of the droplet to channel surfaces occurs, where r (half the height of the droplet, assuming that the height of the droplet is approximately the height of the main channel) is approximately 5 μm and μ (viscosity) of FC-40 is 3.4 cP, solving for U yields a critical velocity of approximately 170 μm/s. These results were in good agreement with experimental estimates made by analysis of videos to determine the lowest velocity achievable before droplets wet to channel walls (100±50 μm/s).

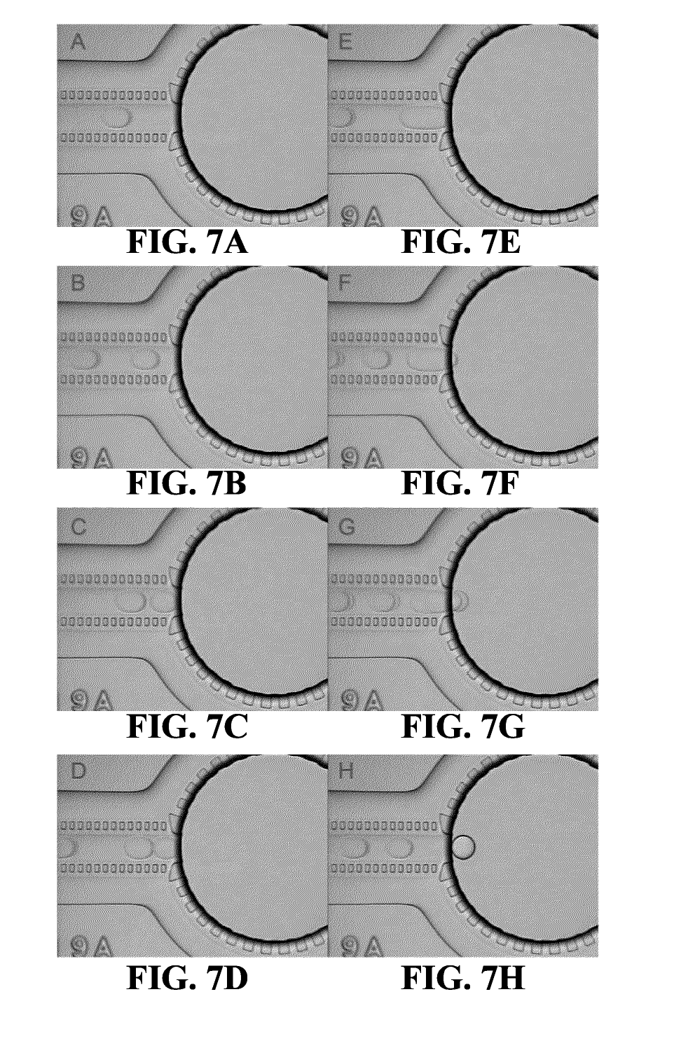

The storage element design allowed for a critical incoming droplet velocities as high as 3.9 mm/s. At the critical incoming droplet velocity, the delivery of droplets to each element in the array took, on average, 7 seconds. Time course images acquired from a video of droplets of water sent to a chamber at a mean flow velocity of 3.9 mm/s through the storage element inlet, equal to the critical incoming velocity, are shown in FIG. 7.

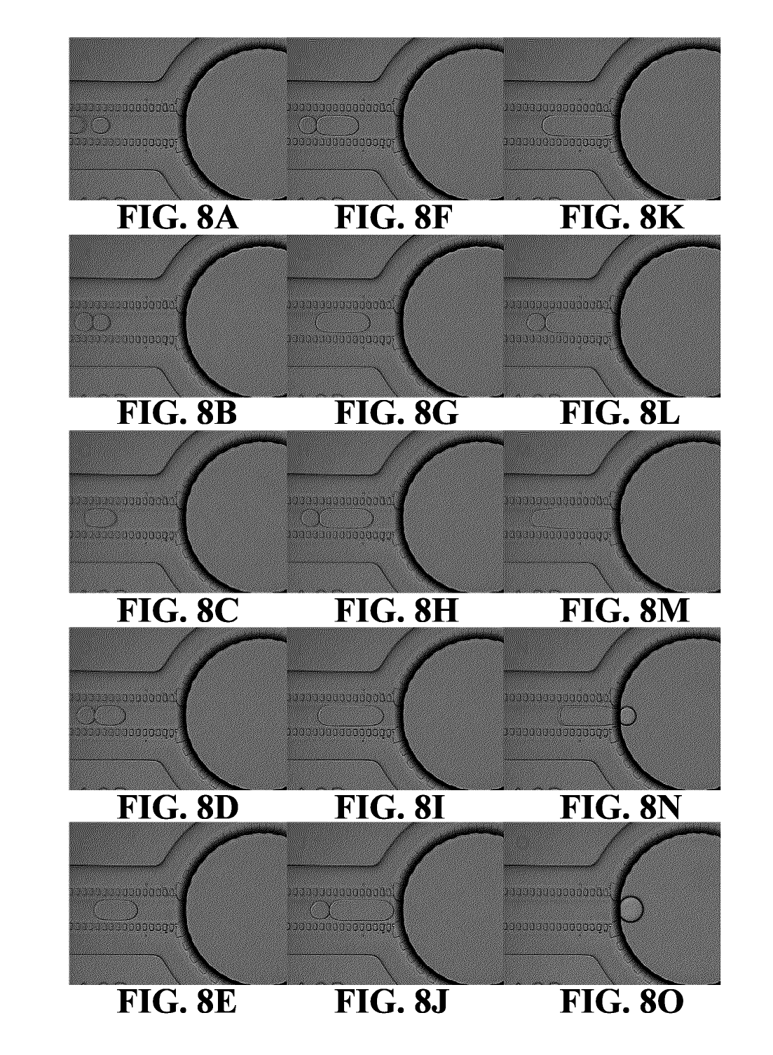

When a droplet is sent to a storage element with a flow velocity below the critical incoming velocity, the droplet is decelerated to the droplet wetting velocity further upstream of the storage chamber. In this case, other droplets must first coalesce with it in order for the merged droplet to reach the edge of the storage chamber, at which point it is pulled in by surface tension. Still images acquired from a video of droplets of water sent to a chamber at a mean flow velocity of 2.9 mm/s through the storage element inlet (less than the critical incoming velocity) are shown in FIG. 8.

This method allows for robust droplet merging regardless of the time between arrivals of multiple droplets at the storage element. When droplets were sent to storage elements at or below the critical incoming droplet velocity, 100% coalescence in 500 events (10 droplets×50 chambers) was routinely observed, both with and without surfactant in the aqueous phase (0.1% Tween 20). Droplets have been merged even after waiting several days between sending droplets to a storage element. Stored droplets also remain at the storage chamber entrance after prolonged heating of the device, permitting additional reagent droplets to be merged with the stored solution after extended heating steps required for many molecular biology protocols.

The volume of the storage chamber defines only an upper limit on the volume of the stored droplet, but the storage element design allows for the formulation and storage of a solution with any volume less than or equal to this limit, allowing for programmable control over the final solution volume.

When operating above the critical incoming droplet velocity, droplets were not sufficiently decelerated by the side channels and entered the retaining chamber without wetting the surface of the main channel. In this case, the free droplets followed an upward trajectory and ultimately came to rest at the chamber ceiling where they wet and were immobilized. Provided that the flow rate was constant, each incoming droplet was delivered to the same location and contacted the previously stored droplets. In the absence of a surfactant in the aqueous (i.e. dispersed) phase, the droplets coalesced shortly after making contact, thereby allowing for the sequential merging of droplets at maximal flow rates. However, when the droplet contents included a surfactant, the coalescence was delayed, leading to transient droplet contact and unreliable merging during the initial additions. However, once the total volume of droplets sent to a storage element was large enough to occupy a significant fraction of the chamber volume (approximately 25%), all droplets merged with the stored droplet. Accordingly, if the final stored droplet volume is sufficiently large and the sequence of droplet merging is unimportant, flow velocities much higher than the critical incoming droplet velocity can be used to achieve faster formulation. Operating in this regime, the storage element can be filled with 100 droplets in approximately 5 seconds.

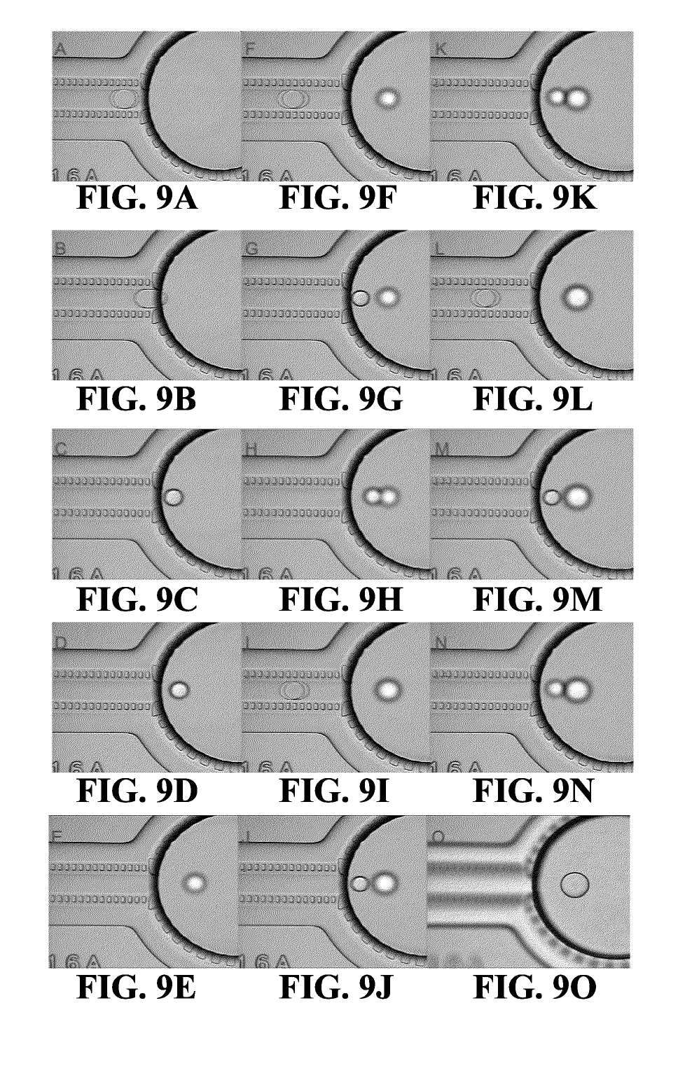

Time course images acquired from a video of droplets of water sent to a chamber at a mean flow velocity of 7.2 mm/s through the storage element inlet (greater than the critical incoming velocity) are shown in FIG. 9. In the last image, the focus has been shifted to the top of the chamber to show that the droplet is positioned at the chamber roof. However, in addition to surfactants in the carrier (i.e. continuous) phase, it is often desirable to include a surfactant in the aqueous (i.e. dispersed) phase to reduce the adsorption of analytes to channel walls in the aqueous section of the device, or to the droplet interface. The inclusion of such surfactants (0.1% Tween 20) has been observed to partially stabilize droplets, significantly increasing the time required for coalescence. Thus, when the droplet contents include a surfactant, a droplet sent into the chamber above the critical incoming velocity has only transient contact with a previously stored droplet, “bouncing” off of it and coming to rest at a different location on the chamber ceiling. Thus, when using aqueous surfactants, the robust merging of each droplet sent to a storage element requires that they be held in contact for an extended time, which can only be ensured by operating at or below the critical incoming velocity to ensure droplet immobilization by wetting. Operating the device using these flow velocities, 100% coalescence in 1000 events (20 droplets×50 chambers) was routinely observed both with and without surfactant in the aqueous phase (0.1% Tween 20).

Stored droplets wetted to the surface of the storage element at the retaining chamber inlet with and without surfactant are shown in FIGS. 10A and 10B, respectively. These droplets were formed by the merging of 20 discrete dispersed phase droplets that were delivered to the main channel below the critical incoming droplet velocity. The aqueous surfactant appears to enhance wetting of droplets onto PDMS surfaces as can be seen by the reduced contact angle in FIG. 9B.

FIG. 11 is a finite element simulation of the flow velocity through the storage element at a height of 2.5 μm (half of the height of a sieve element of this device), and FIG. 12 is a finite element simulation of the flow velocity on the vertical plane through the center of the storage element. Shading indicates flow rate in the storage element decreasing as fluid approaches the retaining chamber. The dark shading toward the entrance of the main channel corresponds to the highest flow rate, while dark shading in the retaining chamber corresponds to the lowest flow rate. Once docked inside the chamber, the droplets are sequestered from high flow and could not be dislodged under the maximum achievable flow velocity (approximately 50 mm/s) through the storage element inlet.

While the encapsulation of reagents into droplets eliminates unwanted diffusion, it also facilitates improved mixing within the droplet. The spherical shape of stored droplets has a diameter smaller than the distances over which reagents must diffuse to achieve complete mixing in typical single-phase microfluidic systems that use a series of interconnected chambers to perform multistep reactions. The time required for complete mixing of a stored droplet by diffusion alone is thus significantly shorter, as the diffusion time of an analyte has a quadratic dependence on the diffusion distance. In addition, the shear stress imparted by the flow of carrier fluid against the stored droplet while transporting droplets to a storage element results in recirculating flows that advectively mix droplet contents, further decreasing mixing times. These factors also allow for small reagent volumes to be added and rapidly mixed with a much larger stored droplet, which is problematic in typical single-phase systems, as the reagents from a small chamber can take a long time to diffusively mix completely into an adjacent chamber with much larger dimensions.

### 1.6 Automated Elution of Selected Droplets

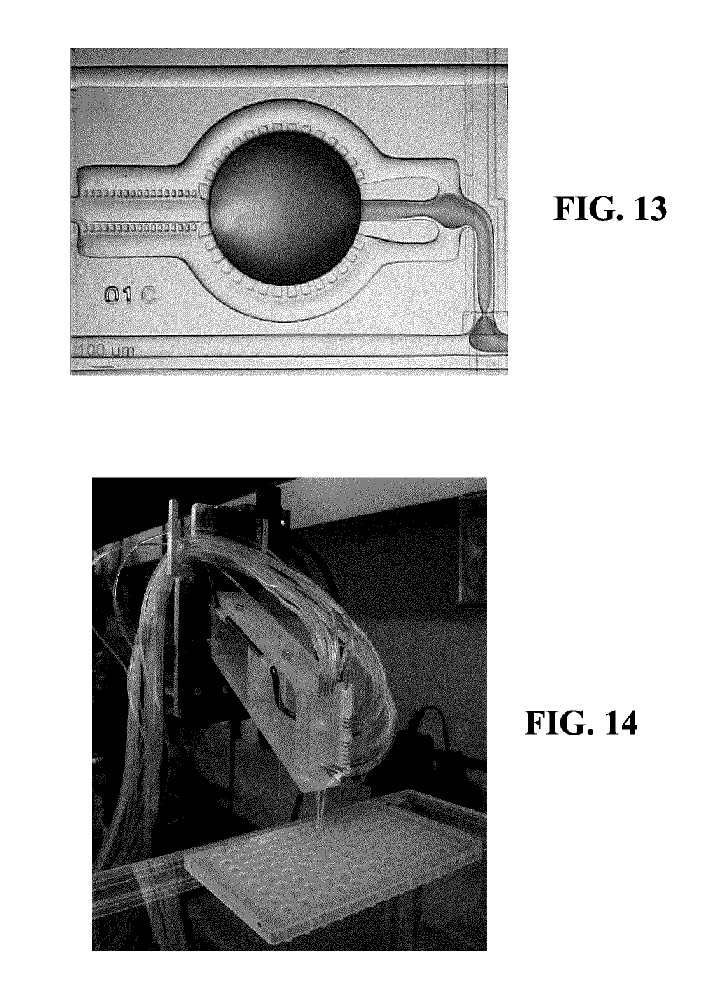

The addressability of the storage chamber array and the compartmentalization of two-phase flow can be exploited to achieve arbitrary elution of individual stored droplets directly into standard microliter-volume tubes in a fully automated manner and with negligible cross-contamination between storage chambers. During elution, equal pressures are applied to one of the aqueous inlets and the oil inlet, which intersect at a T-junction at the reagent-metering module. This results in a continuous oil-sheathed stream of water, which can be directed to any element of the array. The stream coalesces with the stored droplet in the storage element to form an elution stream. If the volume of the elution stream exceeds the retaining chamber volume, the oil-sheathed aqueous stream now containing the stored droplet's contents will be ejected from the element and directed to the elution channel. FIG. 13 is an optical micrograph showing the elution of a retaining chamber using an elution droplet of water encapsulated in oil. The oil surrounding this elution stream minimizes contact between the aqueous (i.e. dispersed) phase and the channel walls, thus minimizing cross-contamination with the contents of other storage elements.

The total aqueous volume used for elution can be controlled by programming the time for which the aqueous phase is allowed to flow into the T-junction. In this embodiment, complete recovery of retaining chamber contents may be achieved by flushing the storage element with approximately 500 nL of water, equivalent to 12.5 times the maximum stored droplet volume. Elution of stored droplets of dye indicates that this volume is sufficient to completely flush the storage element. Deposition of eluted droplets from each chamber was achieved using a zero dead-volume elution nozzle that is designed to fit into standard microfuge tube formats.

To enable automated droplet elution, the device was mounted via a vacuum chuck, as depicted in FIG. 14, to a custom 3-axis robot built from three interconnected precision stages, which allows for automated control of the exact position of the elution nozzle. Custom software is used to calculate the position of each well in any two-dimensional grid based on the position of three corners of the grid that are defined by the user, and enables automated insertion of the elution nozzle into selected wells during elution. Each well is prefilled with light mineral oil, and the tip of the elution nozzle is completely immersed before elution begins. As light mineral oil has a lower interfacial tension with PDMS than both the aqueous and fluorocarbon phases, it coats the opening of the elution channel and the outside surfaces of the elution arm, preventing any of the eluted aqueous phase from adhering to the exterior nozzle surfaces and contaminating the next well that the nozzle is lowered into. As light mineral oil also has a lower density than water, aqueous droplets expelled from the elution channel sink to the bottom of the well to minimize the chance of aqueous adhesion onto the nozzle. Elution with a volume of ˜5 μL is sufficient to ensure that any aqueous droplets that may remain on the nozzle exterior at the end of the elution process contain a negligible amount of stored droplet contents.

After elution of an addressed storage chamber is complete, channels of the array that are in the elution path of other storage chambers may be similarly flushed with an aqueous stream to ensure that any possible contaminant droplets are expelled. As a final precaution, after elution of each storage element, the nozzle may be rinsed in an isopropanol bath to wash away any aqueous droplets that may remain attached to the nozzle's exterior and can lead to sample carry-over. Isopropanol may be chosen because it dissolves light mineral oil, thus allowing aqueous droplets on the nozzle exterior, which may be encased in light mineral oil, to be washed away. After elution, additional aqueous buffer can be added to the tubes to obtain a larger volume for handling by pipette, and the tubes centrifuged to ensure coalescence of all aqueous components.

### 1.7 Formulation Performance

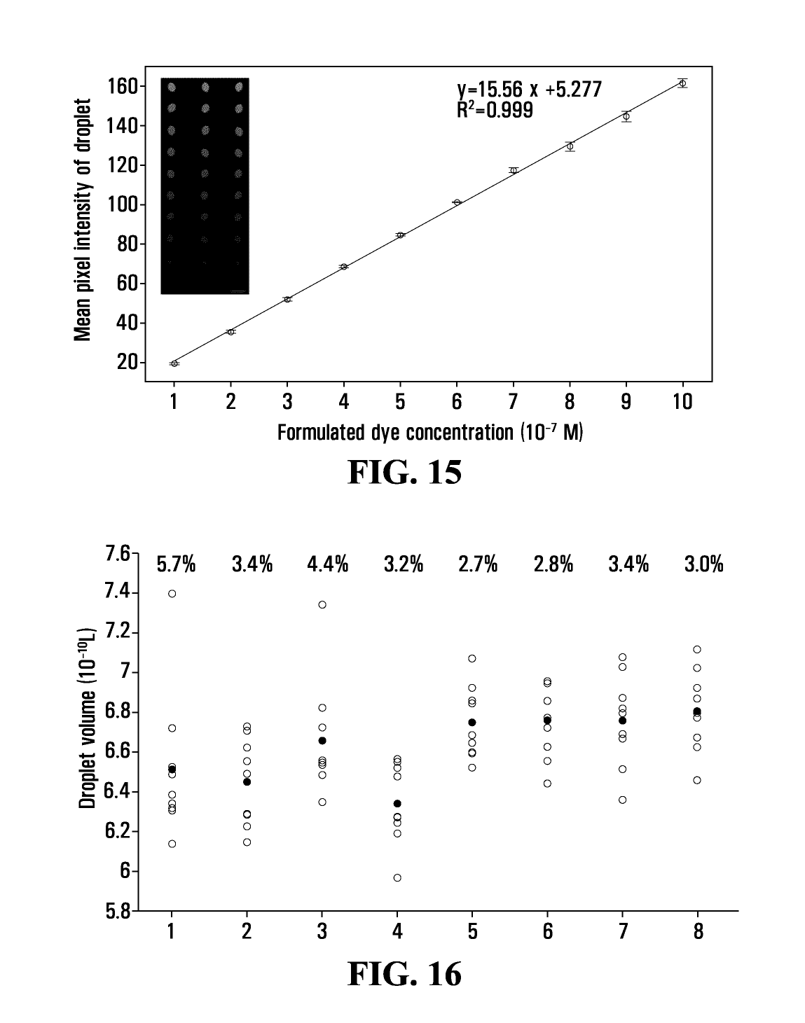

To establish the formulation accuracy of the microfluidic method and device, a series of 26.6 nL stored droplets, having ten different fluorescent dye concentrations ranging from 100 nM to 1 μM, was formulated by dispensing programmed numbers (200 in this case) of pump increments of 1 μM dye or a diluting buffer. As shown in FIG. 15, the resulting dye concentrations, as measured by mean fluorescent intensity, were found to be in agreement with target values over the full range (R²=0.999), with an average coefficient of variation of 1.4%. The inset of FIG. 15 shows a corresponding fluorescent confocal image of the array of stored droplets, with a scale bar representing 1 mm.

Metering precision was also evaluated by delivering 5 pump increments of water from each of the 8 aqueous inlets to 9 storage elements located across the array. The stored droplet volume in each of the 72 storage elements was determined by microscopy and image analysis. FIG. 16 is a scatterplot showing the measured volumes of stored droplets in 9 different retaining chambers loaded from each of the 8 reagent inlets. The mean stored droplet volume is depicted by solid circles, with the standard deviation indicated at the top. For all 72 elements, the average absolute volume of a pump increment was determined to be 133 pL with a standard deviation of 4.8% (FIG. 16), which was near the accuracy of imaging measurement.

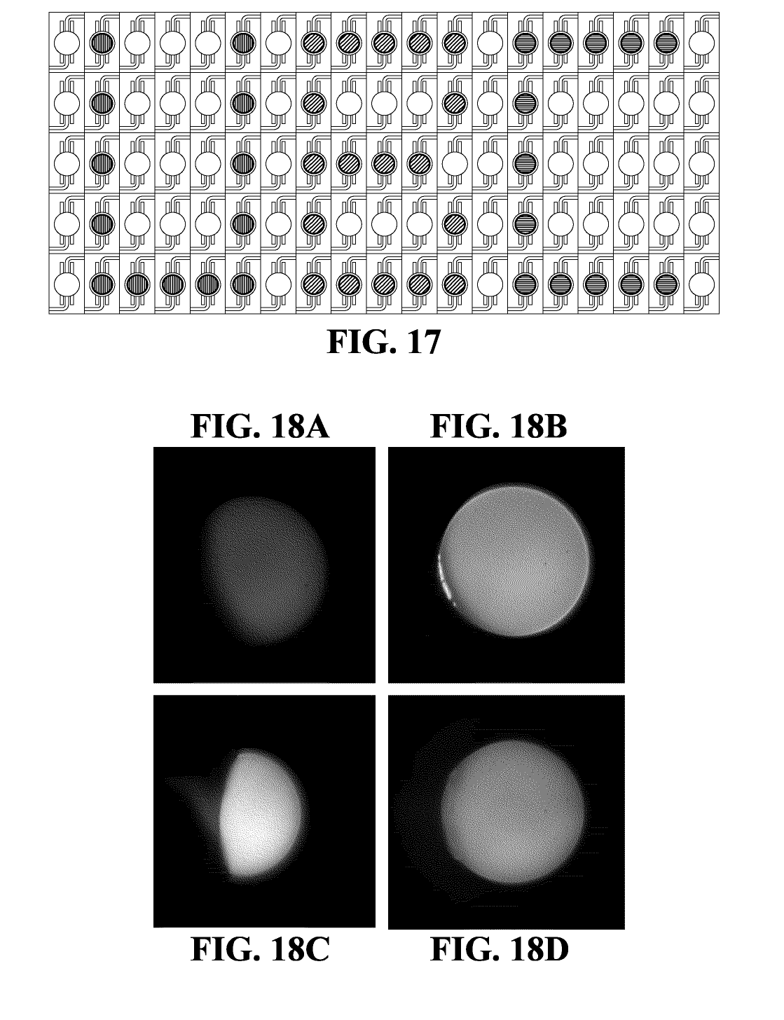

As a demonstration of arbitrary and addressable formulation, the device was utilized as a programmable display as shown in FIG. 17. Stored droplets formed from the combination 300 pump increments were arranged three letters on the storage array using a two-fold dilution series of three colored dyes from top to bottom of each letter. The droplet metering unit is visible at the bottom right of the micrograph.

To determine the extent to which protein adsorption to droplet interfaces occurs in the present device, stored droplets of fluorescein isothiocyanate (FITC)-labeled bovine serum albumin (BSA) and Alexa 488-labeled fibrinogen in phosphate buffered saline (PBS) were fluorescently imaged using different surfactants added to the fluorous and aqueous phases. BSA and fibrinogen are known to adsorb to a wide variety of surfaces and are often used as test proteins in studies of protein adsorption. Four surfactant combinations were tested: PFO mixed with FC-40 (1:5 v/v ratio) as the carrier phase with and without 0.1% Tween 20 surfactant added to the aqueous phase, and the OEG-capped fluorosurfactant mixed with FC-40 (1:4 v/v ratio) as the carrier phase with and without 0.1% Tween 20 added to the aqueous phase. The latter fluorosurfactant was extracted from Zonyl FSO-100.

Fluorescent images of stored droplets of FITC-labeled BSA are shown in 18A, 18B, 18C, and 18D. The PFO/Tween 20 combination was tested first with a 100 nM solution of BSA (FIG. 18A). No apparent adsorption to the droplet interface was observed, as fluorescent intensity fades towards the edge of the droplet. Next, the BSA solution without Tween 20 using the same carrier fluid was imaged, and fluorescence was virtually undetectable using identical camera exposure and gain settings. A 10× increase in BSA concentration to 1 μM was required to obtain comparable fluorescent intensity (FIG. 18B), providing evidence of BSA adsorption to PDMS channel walls before encapsulation into droplets. Moreover, adsorption to the droplet interface is clearly visible in FIG. 18B as a ring of increased fluorescent intensity at the edge of the droplet. The contrast between FIGS. 18A and 18B indicate that inclusion of Tween 20 prevents protein adsorption to droplet interfaces. Using another device, the OEG-capped fluorosurfactant/Tween 20 combination was tested, again using a 1 μM BSA solution (FIG. 18C). No apparent adsorption to the droplet interface was observed. However, this fluorosurfactant was noticeably less effective than PFO at preventing droplet adhesion onto PDMS walls during both transport and storage. During transport, this resulted in the occasional break off of satellite droplets. The BSA solution without Tween 20 was then imaged (FIG. 18D), and fluorescent intensity of the droplet decreased relative to the solution with Tween 20, again confirming that Tween 20 prevents adsorption to PDMS channel walls. In contrast with the test case using PFO, no BSA adsorption to the droplet interface is seen.

Similar results for the above surfactant combinations were obtained with Alexa 488-labeled fibrinogen. Although only BSA and fibrinogen were tested, these results suggest that the inclusion of 0.1% Tween 20 in the aqueous phase prevents protein adsorption to both PDMS surfaces and fluorous/aqueous droplet interfaces for both fluorosurfactants tested. Considering that PFO was observed to be superior to OEG-capped fluorosurfactant at preventing unwanted droplet adhesion to channel walls, it was concluded that the addition of PFO to the carrier phase and Tween 20 to the aqueous phase prevents unwanted protein adsorption while maintaining desired droplet-based fluid-handling functionality

1.8 on-Chip Quantitative PCR (qPCR) of Formulated Template Dilutions.

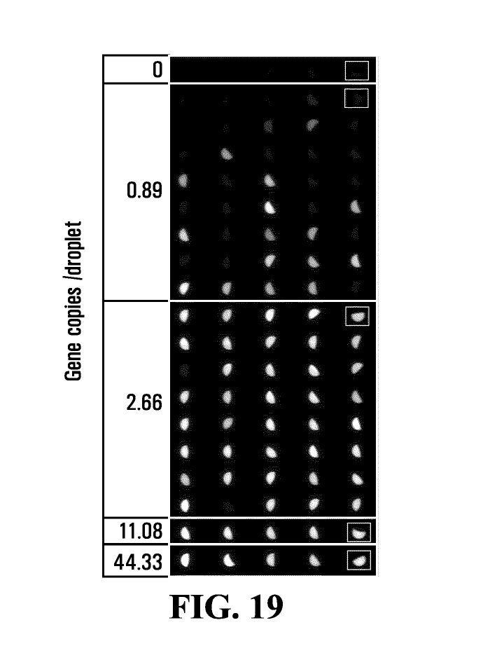

Droplet-based microfluidics is of considerable interest in genomics applications where small volume compartmentalization has been shown to increase analysis sensitivity and precision (38). To validate the effectiveness of a storage element for nucleic acid processing, qPCR of template dilutions formulated on-chip was performed on 90 stored droplets of varying template concentration. 100 pump increments (approximately 13.3 nL) of either human genomic DNA (gDNA) or water were combined in chambers to formulate template dilutions of 44.33 (133 pg, N=4), 11.08 (33.25 pg, N=4), 2.66 (7.98 pg, N=39), 0.89 (2.66 pg, N=39), and 0 (N=4) haploid genome copies per reaction (assuming 3 pg per haploid genome). PCR reactions were then assembled by dispensing droplets of PCR master mix to each retaining chamber, including primers and a hydrolysis probe designed for the detection of the RNase P gene which is present at a single copy per haploid genome. Following reaction assembly, the device was thermocycled on a microfluidic qPCR instrument and fluorescent images were acquired at each cycle. The final fluorescent image of the droplet array after the last cycle is shown in FIG. 19. The expected haploid equivalents per droplet are indicated on the left. Rectangles denote control reactions mixed off-chip. The scale bar represents 1 mm. A digital pattern of amplification can be seen in the lowest two template concentrations. The shape of the stored droplets is non spherical due to wetting of the PCR solutions onto the chamber walls.

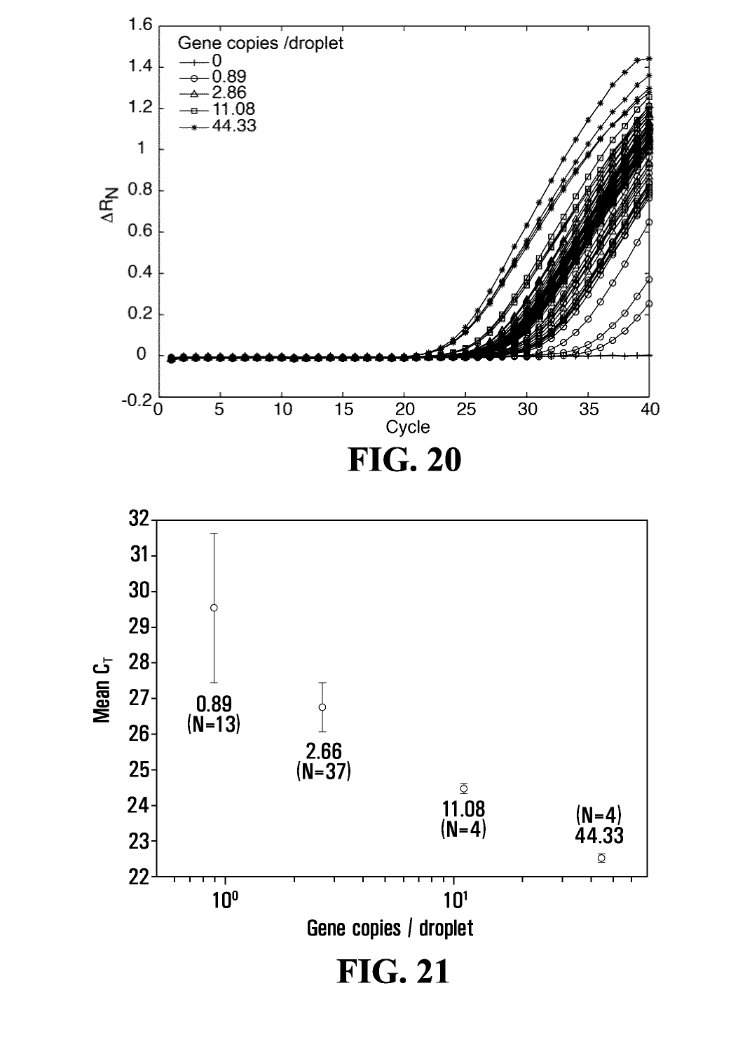

Images were processed using custom image analysis software to generate real-time amplification curves for each droplet as reported in FIG. 20. Measured cycle threshold (CT) values are reported in FIG. 21, with error bars representing the standard deviation across all replicates. CT values for the two highest dilutions were 22.56 (SD=0.12) and 24.49 (SD=0.14) respectively, corresponding to an absolute precision in concentration measurement of 9.4%, which is near the limit of qPCR. The difference in CT (ΔCT) corresponding to a 4 fold dilution was found to be 1.93±0.18, indicating a PCR efficiency of 96.5%. At the two lowest dilutions, digital patterns of amplification were observed, with 37/39 (95%) positive chambers at 2.66 genome equivalents per chamber, and 18/39 (46%) positive chambers at 0.89 genome equivalents per chamber. Assuming a binomial distribution with the occupancy of each chamber determined by Poisson statistics, the expected frequencies at 2.66 and 0.89 genome copies per chamber are 93% and 59% respectively. The observed frequencies fall within symmetric 95% binomial confidence intervals constructed around these frequencies: 79% -98% for 2.66 copies per chamber, and 42%-74% for 0.89 copies per chamber. These results indicate that the device is suitable for PCR-based analysis of limited samples with high efficiency and single molecule sensitivity.

### 1.9 Quantification of Cross-Contamination During Formulation and Elution.

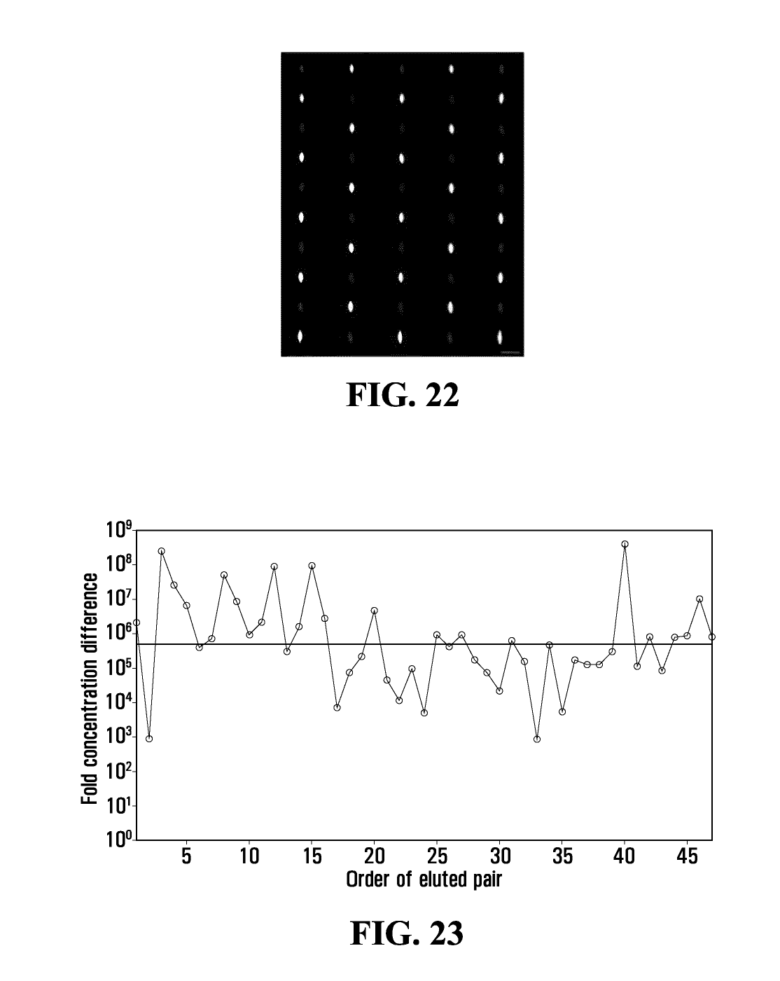

The above qPCR assay was used to quantify cross-contamination between sequentially loaded chambers. 50 chambers were alternately loaded in a checkerboard pattern, each receiving 100 pump increments (approximately 13.3 nL) of PCR reagents premixed with either genomic DNA (approximately 1476 genome equivalents) or no template control (NTC). This checkerboard pattern maximizes the shared fluidic path length to storage chambers with different contents, thus making this test of cross-contamination as stringent as possible. FIG. 22 is a micrograph of the endpoint fluorescent image after 40 cycles of PCR which shows that all positive chambers were successfully amplified while no NTC chambers amplified, indicating that no detectable cross-contamination occurred during loading. Based on the demonstrated ability to detect a single copy of the target gene, the upper boundary for cross-contamination is determined to be 1 in 1476.

Next, cross-contamination during elution of stored droplets was measured. 47 chambers were first loaded with 100 pump increments of water and another 47 were then loaded with an equal volume of qPCR solution containing DNA template (18 genome equivalents) in a checkerboard pattern of alternating water and PCR droplets. Following 40 cycles of on-chip PCR amplification, pairs of PCR product and water droplets were alternately eluted from the device into separate microfuge tubes. Each sample was then diluted in 20 μL of water, and 2 μL of this was used as template in off-chip qPCR reactions to assess relative quantities of eluted template. ΔCT between PCR and water reactions was computed, and the relative fold concentration difference was calculated as 2^{ΔCT}, corresponding to the degree of carry-over between wells. The fold concentration difference of template in eluted pairs of droplets containing amplified template and water is shown in FIG. 23, with the horizontal line denoting the mean. The absolute mean fold concentration difference for all eluted pairs was 4.84×10⁵ with a standard deviation of 19.8. This low level of cross-contamination is acceptable for even the most stringent downstream analyses including sequencing, cloning, or gene expression profiling.

Elution of storage chambers was tested by acquiring fluorescent images of a 40 nL stored droplet of 5 μM fluorescein-labeled 40-mer oligonucleotides before and after elution with ˜500 nL of water. A separate chamber filled with water to an equal volume was then imaged for comparison. The mean fluorescent intensity, measured by ImageJ software, of the water-filled chamber was subtracted from that of the eluted chamber in order to account for background fluorescence, and the result was found to be 0.16% of the oligonucleotide-filled chamber before elution, indicating 99.84% sample recovery. Three other water-filled chambers of equal volume were also imaged to measure the noise of the imaging measurement. The coefficient of variation was found to be 1.7%.

In another embodiment, at least 5 μL of water may be used to elute each chamber in order to ensure that storage chamber contents do not remain attached to the elution nozzle exterior but sink to the bottom of the light mineral oil-filled microfuge tube. As this is a 10-fold increase relative to the volume used for the elution test described above, complete elution of chamber contents is virtually guaranteed.

### 1.10 Single Bacterial Sorting and Culture.

As the analysis of genetic and expression variation at the single cell level has been shown to yield significant biological insight, the ability to isolate and perform a variety of assay protocols on single cells is highly desirable in any programmable microfluidic system. Droplets are particularly well-suited to the isolation and manipulation of bacteria (39; 40) which, due to their small size, are otherwise difficult to manipulate using hydrodynamic trapping mechanisms on-chip. The integrated cell sorter in the present embodiment allows morphological or fluorescence-based sorting of single cells into droplets that can be directed to any chamber. This capability was exploited to perform a variety of experiments on sorted single bacteria.

### 1.10.1 Phenotype-Based Sorting of Single Cells.

To demonstrate the capabilities of the device for phenotype-based sorting of single cells from a mixed population and monoclonal bacterial culture, single bacteria from a suspension containing two strains of Salmonella typhimurium SL1344 (41) expressing green fluorescent protein (GFP) or red fluorescent protein (RFP) were sorted and cultured.

Prior to on-chip bacterial culture, GFP and RFP-expressing strains of Salmonella typhimurium SL1344 with an ampicillin resistance gene were each first aerobically cultured in 2 mL of LB broth (Sigma Aldrich™) with 100 μg/mL ampicillin for ˜18 hrs at 37 C. to reach stationary growth phase (˜10⁹ cells/mL) For each strain, 2 mL of fresh culture media was then inoculated with 6 μL of cell culture and incubated for another 2 hrs to produce exponential growth phase cultures, which were mixed in a 1:1 ratio and diluted to a concentration of ˜1 cell/10 nL prior to single cell sorting on-chip to ensure that no more than one cell was present at any given time in the channel intersection, which had a volume of ˜300 pL. The concentration of the stationary growth phase cultures of both strains were measured by absorbance (OD 600) to be equivalent, and 10× dilutions of these cultures in culture media were then used to seed the on-chip multiple cell cultures. Suspensions of K12 Escherichia coli bacteria (ATCC 10798) used were cultured and prepared as above, but without ampicillin in the media, and were stained with SYTO 9 DNA stain (Invitrogen™) prior to use on-chip. For on-chip PCR and WGA experiments, bacterial cultures were resuspended 3 times in PBS to remove free DNA from the suspension fluid prior to use on-chip.

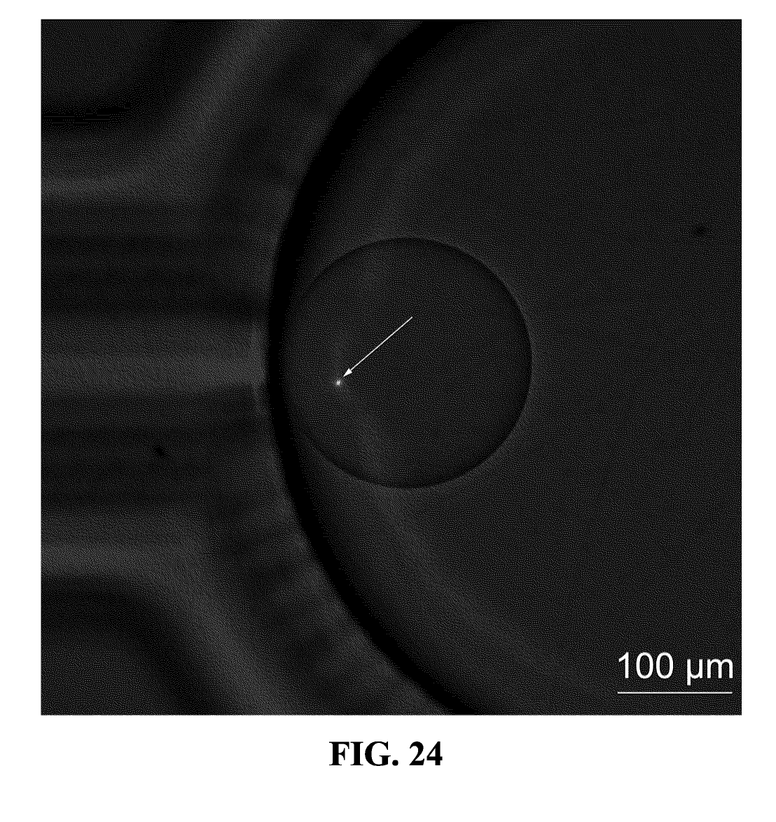

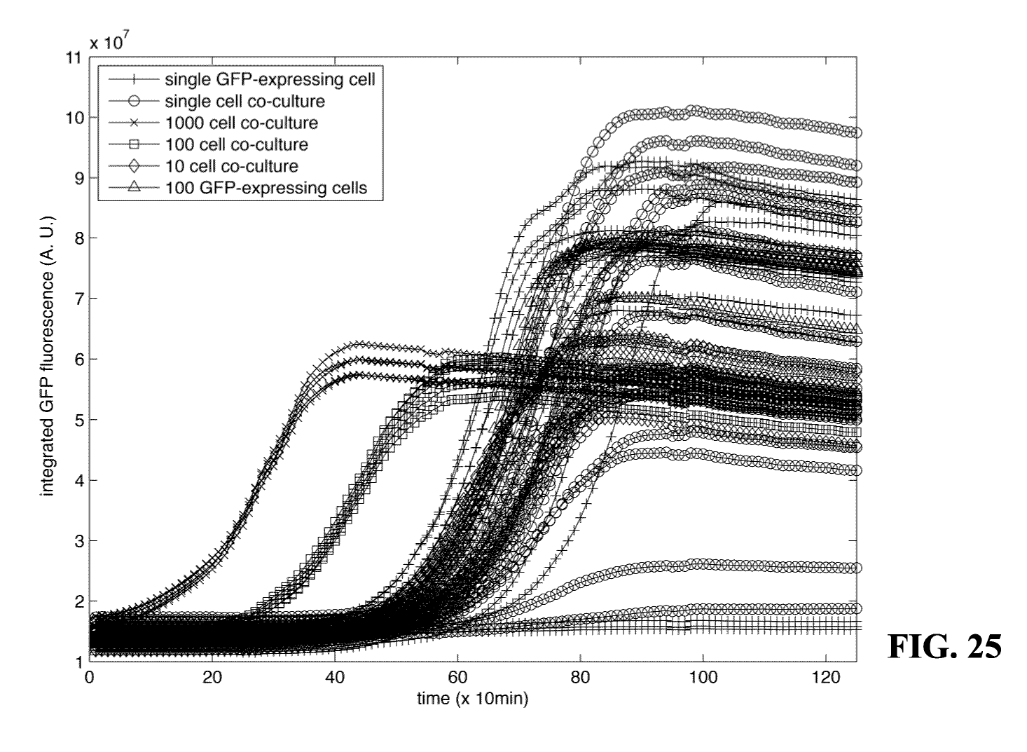

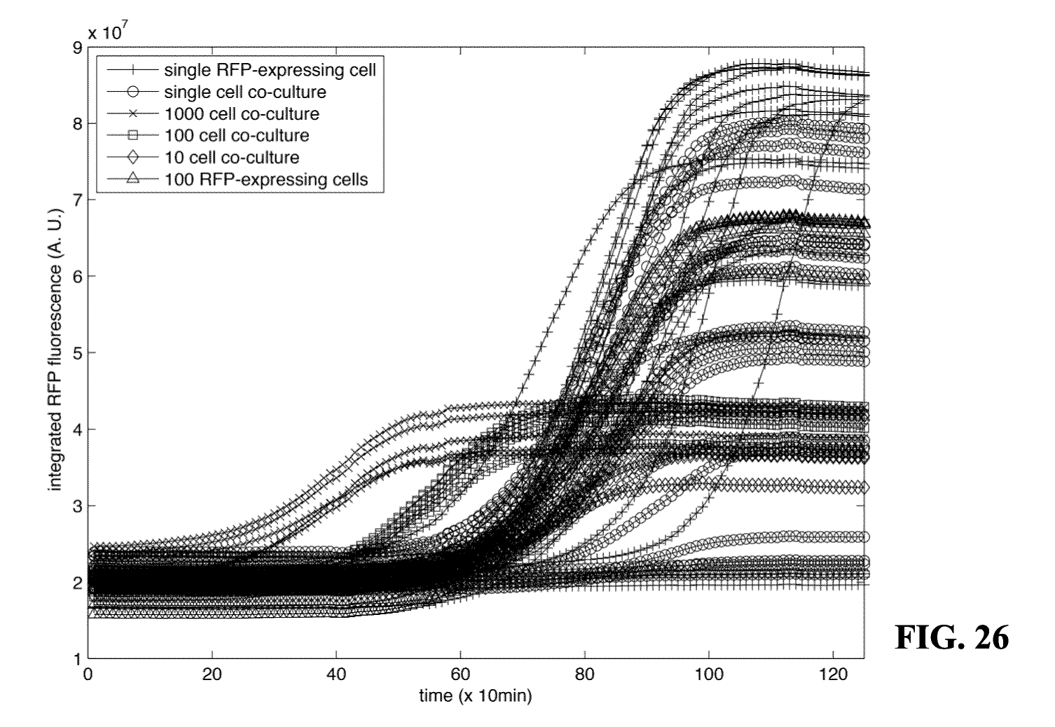

Filter cubes for the detection of GFP and RFP were used to identify cells of each strain. Using the cell sorter, 20 single cells of each strain were each sorted into individual storage elements to seed monoclonal cultures in a checkerboard pattern on the array. FIG. 24 is an image of overlaid brightfield and fluorescent micrographs of a single RFP-expressing salmonella in a stored droplet. The bright spot at the tip of the arrowhead is the bacterium. A single cell of each strain was also sorted into the same storage element to seed 20 single cell co-cultures. Single strain cultures were also seeded with approximately 100 cells (N=5), as well as co-cultures with approximately 10 (N=5), 100 (N=5), and 1000 (N=5) cells of both strains. After loading cells into storage elements, they were filled with growth media in droplets dispensed from a separate reagent inlet to a final volume of approximately 40 nL. The device was then placed on the microfluidic qPCR instrument as above, and the temperature was set to 25° C. with GFP and RFP-channel fluorescent images acquired every 10 minutes for 23.3 hours. Images were processed using custom image analysis software to generate growth curves based on measured GFP and RFP expression for each culture. The growth curves for GFP- and RFP-expressing cells are shown in FIG. 25 and FIG. 26, respectively.

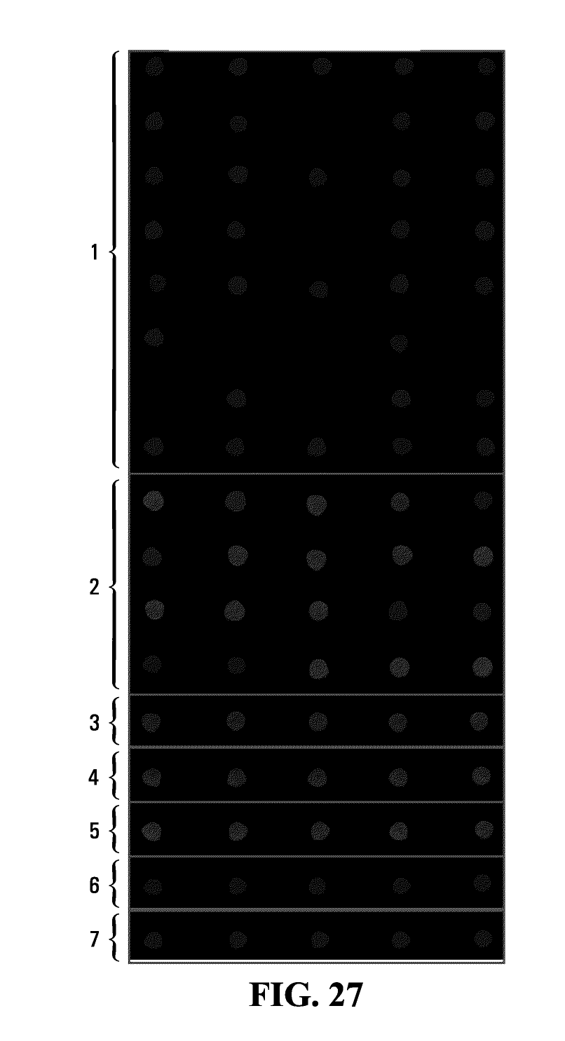

After the incubation period, the droplet array was imaged with a confocal scanner. GFP- and RFP-channel confocal scans of all cultures in the array after incubation are shown in FIG. 27. Scale bar is 1 mm Cultures were seeded with: (1) single cells (dark parts of the array are unsuccessful cultures). Storage elements in each row were alternately seed with GFP- or RFP-expressing strains, such that each successful culture is distinctive read or green; (2) a single cell of each strain. The single-cell co-cultures exhibit a random distribution, with one strain or the other occasionally dominating the culture such that the culture is purely red or green; (3) ˜1000 cells of each strain. The cultures are not dominated by one strain or the other, such that none of the cultures are purely red or green; (4) ˜100 cells of each strain. The cultures are not dominated by one strain or the other, such that none of the cultures are purely red or green; (5) ˜10 cells of each strain. The cultures are not dominated by one strain or the other, such that none of the cultures are purely red or green; (6) ˜100 GFP-expressing cells. Each culture is purely green; and (7) ˜100 RFP-expressing cells. Each culture is purely green.

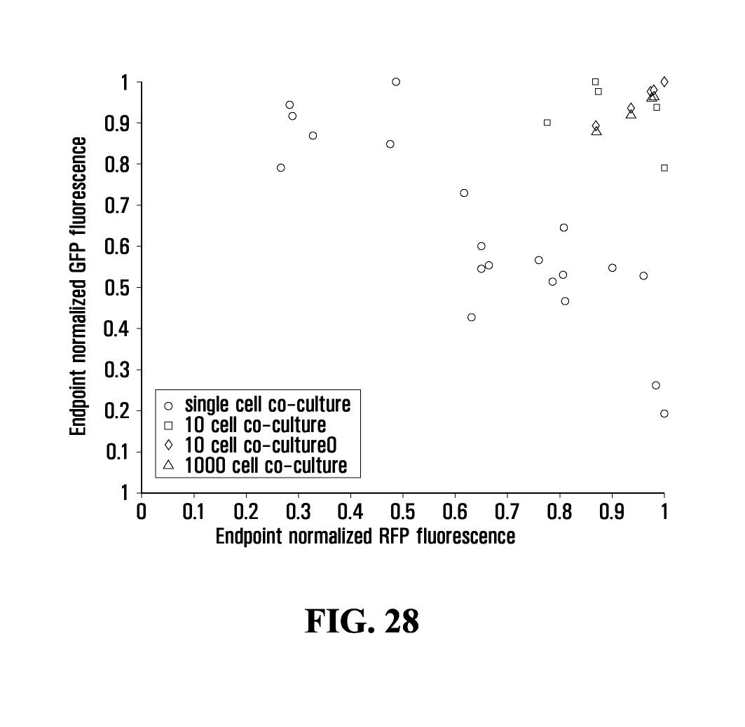

17 of 20 (85%) and 16 of 20 (80%) of the GFP and RFP-expressing monoclonal cultures (respectively) grew successfully. No GFP fluorescence was detected in the RFP-expressing monoclonal cultures and vice versa, indicating contamination-free cell sorting. The ability to load varying numbers of cells of each strain into the same culture enables the examination of stochasticity in competitive co-cultures. FIG. 28 is a scatterplot showing normalized endpoint intensity for GFP- and RFP-channels for co-cultures seeded with different numbers of both cells. As can be seen, the single-cell co-cultures exhibit a random distribution with one strain or the other occasionally dominating the culture. However, as the seeded cell number of each strain increases, this stochastic effect is lessened and the data points converge to the diagonal.

### 1.10.2 PCR-Based Genotyping of Single Bacteria.

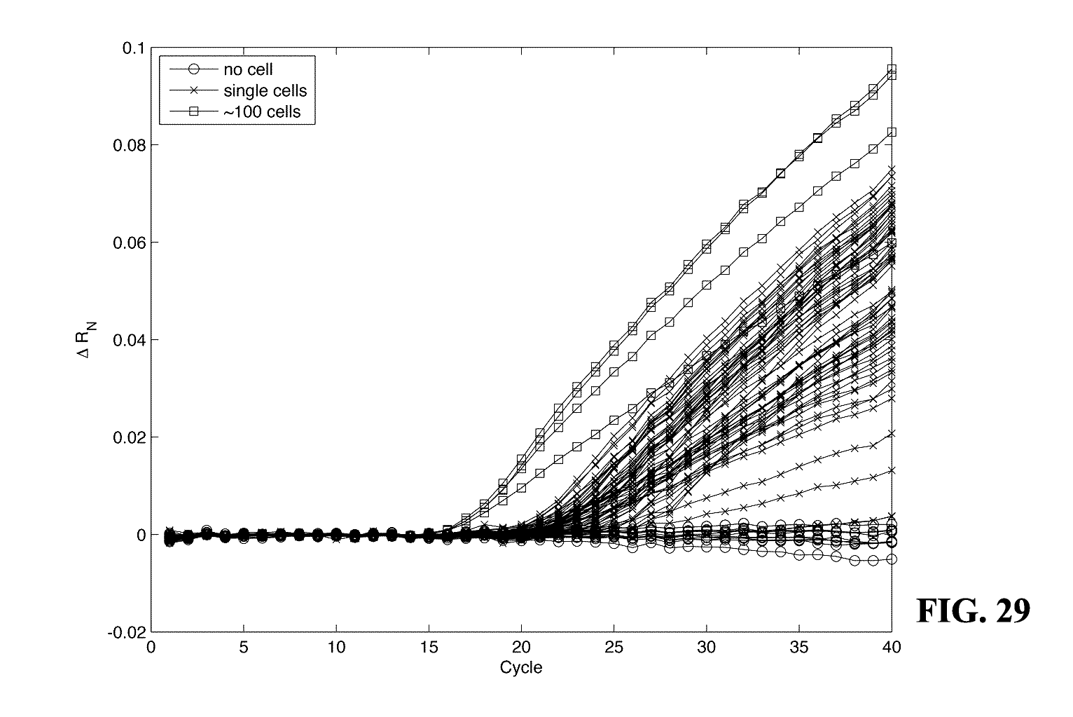

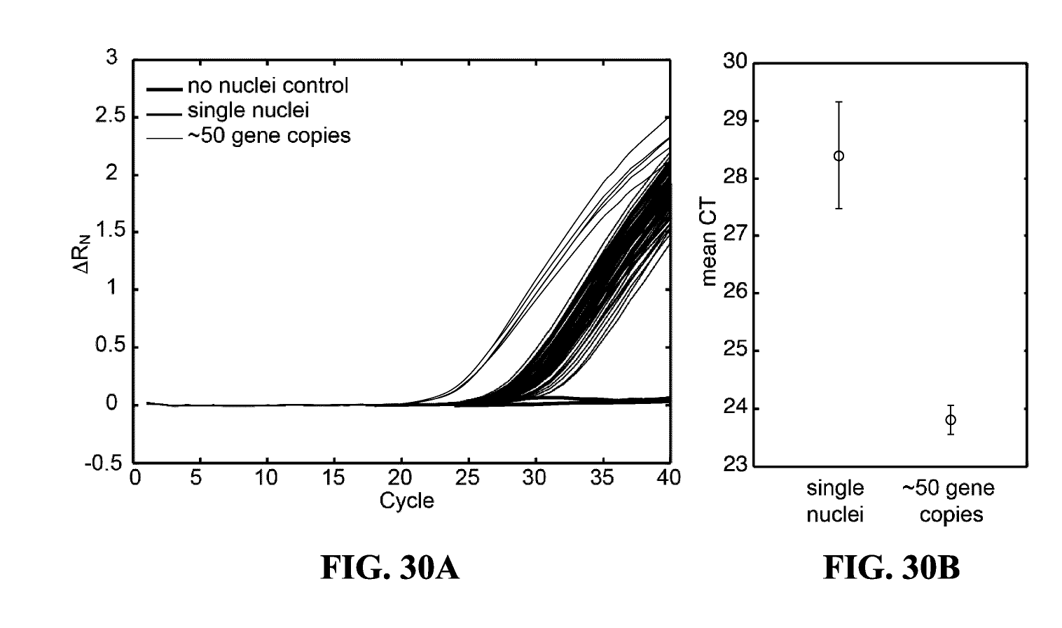

The genetic analysis of single cells is emerging as a necessary pursuit for the understanding of complex biological systems. Perhaps nowhere is this more important than in the microbial domain where each cell is a distinct organism, and where in vitro culture for clonal expansion is often not possible. To demonstrate the use of this device for the genetic analysis of single bacteria, PCR-based genotyping of single K12 Escherichia coli bacteria sorted from suspension was performed. 62 storage elements were loaded with single cells, 5 storage elements were loaded with approximately 100 cells in exponential growth phase, and 10 storage elements were loaded with no cells (as determined during cell sorting). All storage elements were then loaded with PCR master mix, including primers designed for the detection of a strain-specific fragment of the 16S rRNA gene (present at 7 copies per genome in E. coli), and an intercalating dye. Following reaction assembly, the device was thermocycled on the microfluidic qPCR instrument, and qPCR curves were generated for each droplet. The qPCR curves are shown FIG. 29. CT values, shown in FIG. 30, were calculated from the qPCR curves (error bars represent standard deviation). The target sequence was successfully amplified in 60 of 62 (97%) single cells, 4 of 5 (80%) multiple cell reactions, and none of the no-cell control reactions, confirming that the device allows for single bacterial analysis without contamination between reactions. The ΔCT between the single and 100-cell reactions was found to be 6.52±2.06, indicating an assay efficiency of 98%.

Following qPCR, the amplicon from each on-chip reaction was eluted, further amplified in a standard microlitre-scale reaction, and gel purified to obtain sufficient DNA mass for sequencing. Ten of the single cell reaction amplicons were randomly chosen for capillary sequencing, the results of which verified that the correct sequence had been obtained for all ten reactions.

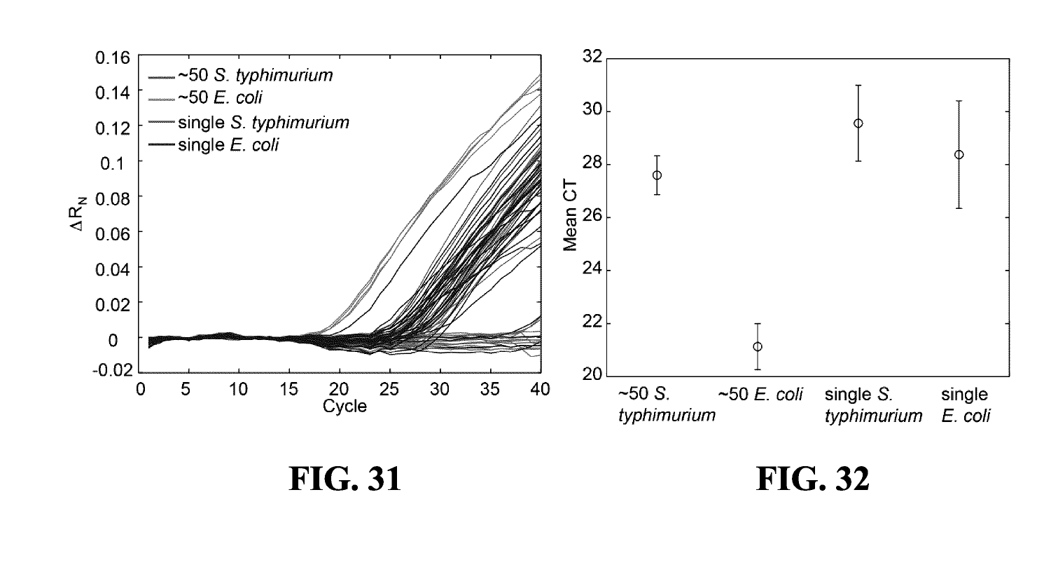

In many applications, it can be important to identify bacterial species present in a mixed population. While species-specific assays as used above can be employed to detect the presence of a single target species in a large background (39), it may sometimes be desirable to detect multiple species or to identify unknown members of a sample. For such applications, species-specific assays are ineffective. A better strategy is to use a single assay to amplify a genomic region whose sequence can be used for identification. As a demonstration of this, genotyping experiments based on PCR amplification and sequencing of the 16S rRNA gene were performed on single bacteria sorted from a mixed population of Escherichia coli (E. coli) and RFP-expressing S. typhimurium. E. coli cells were stained with fluorescent SYTO9 DNA stain, which fluoresces in the GFP channel, in order to distinguish them from RFP-expressing S. typhimurium by fluorescence microscopy. Storage elements were loaded with single S. typhimurium (N=30), single E. coli (N=29), ˜50 S. typhimurium (N=5), and ˜50E. coli (N=5), and mixed with PCR reagents containingan intercalating dye and primers targeting a 144-bp segment of the 16S rRNAgene. The sequence of this segment differs by four single base pair mismatches between the two species. On-chip qPCR was performed, with an initial 3-minute heating step at 95 C. included to perform heat lysis of bacteria, and qPCR curves for all reactions were constructed from acquired images (FIG. 31).

The target sequence was amplified in 16 of 30 (53%) single S. typhimurium, and 25 of 29 (86%) single E. coli, as determined by qPCR curves for each reaction. The difference in mean CT between single and ˜50-cell reactions was 1.96 and 7.24 for S. typhimurium and E. coli respectively (FIG. 32), indicating sub-optimal PCR efficiencies of 71.7% and 636% respectively. Following PCR, the amplicons from each reaction were eluted and six successful single-cell reactions from each species were chosen at random for further off-chip amplification and capillary sequencing. Based on the sequence data at the four mismatched positions of the 144-bp amplicon, all six single E. coli cells and five of six single S. typhimurium cellswere correctly identified. The single S. typhimurium amplicon that could not be identified also did not match the expected sequence for E. coli. These results demonstrate the ability to use the method to identify single sorted bacteria from a mixed population using a single assay and sequencing of the amplicon.

### 1.10.3 Single Cell Whole Genome Amplification.

As a further demonstration of the flexibility and programmability of the method's cell processing capabilities, a multi-step whole genome amplification (WGA) procedure was performed on 127 single E. coli cells using two devices. No-cell control reactions (N=20) and reactions on approximately 10 cells (N=10) and 1000 cells (N=10) were also performed.

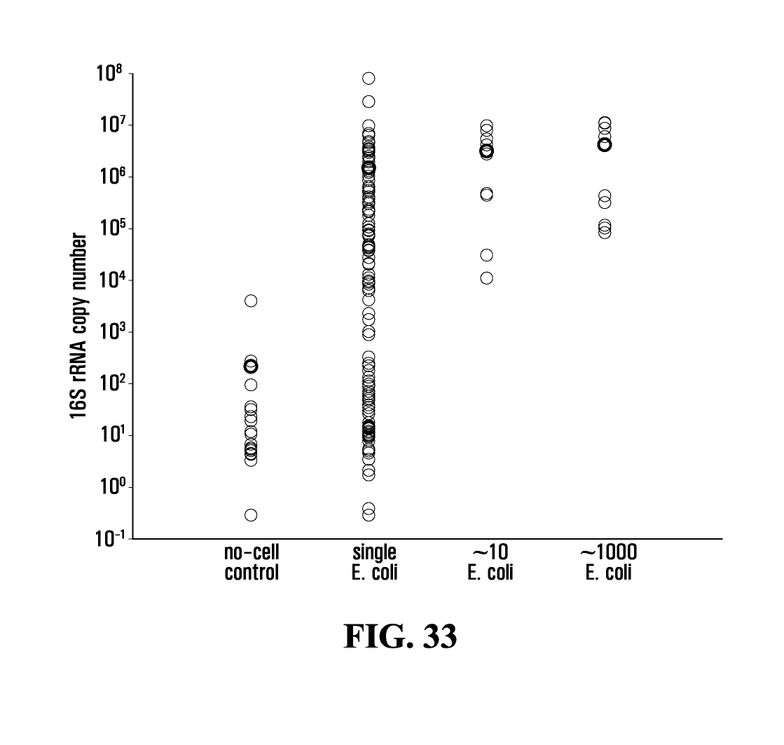

Thermocycling steps for on-chip WGA was performed by placing the device on a flatbed thermocycler and taping the device to the heating surface to ensure good thermal contact. Thermocycling protocols recommended by the manufacturer were used. To quantify on-chip WGA-amplified E. coli DNA, eluted sample was diluted into 20 μL of water, 2 μL of which was used in an off-chip qPCR reaction using the K12 E. coli-specific assay above. CT values were compared to those from a standard curve generated from qPCR reactions on dilutions of purified E. coli gDNA (ATCC) with known 16S rRNA copy number (7 per genome; FIG. 33). The dilution factor during device elution was taken into account to quantify on-chip amplification. Reactions containing no cells, single cells, ˜10 cells, and ˜1000 cells produced mean copy numbers of 220, 2.5×10⁶, 3.2×10⁶, and 4.3×10⁶ respectively. The coefficient of variation of copy number in all single-cell reactions was 507%, and 72 of 127 (57%) single-cell reactions resulted in at least a 100-fold amplification of the 16S rRNA gene relative to the 7 copies present in a single cell. This metric for successful single cell WGA is a stringent one given that known biases in WGA chemistry may result in amplification of genomic regions other than the one targeted by our assay.

Product from six successful single cell reactions, two no-cell control reactions, and one approximately 1000-cell reaction were chosen for further analysis by pyrosequencing using an Illumina Genome Analyzer™ 2 platform. Purified unamplified gDNA was also sequenced as a positive control. Sequencing libraries for each single cell were constructed from reaction product eluted directly from the chip as well as after further amplification in a microlitre-scale reaction.

Bacterial PCR amplicon from each on-chip reaction was first eluted and diluted into 20 μL of water, 2 μL of which was used as template in an off-chip PCR reaction. This amplicon was then run on an agarose gel, the band was cut out, and DNA extracted using a Qiagen Qiaguick™ Gel Extraction kit. DNA was then sequenced using an Applied Biosystems™ 3730S 48-capillary DNA Analyzer with POP-7 BigDye® Terminator v3.1 sequencing chemistry. Sequencing data was analyzed using CLC Bio Main Workbench™ software. The expected sequences of the fragment amplified by the E. coli/Salmonella 16S rRNA assay in E. Coli and Salmonella (respectively) are:

TCGTGTTGTGAAATGTTGGGTTAAGTCCCGCAACGAGCGCAACCCTTATC
CTTTGTTGCCAGCGGTCCGGCCGGGAACTCAAAGGAGACTGCCAGTGATA
AACTGGAGGAAGGTGGGGATGACGTCAAGTCATCATGGCCCTTA
and
TCGTGTTGTGAAATGTTGGGTTAAGTCCCGCAACGAGCGCAACCCTTATC
CTTTGTTGCCAGCGGTTAGGCCGGGAACTCAAAGGAGACTGCCAGTGATA
AACTGGAGGAAGGTGGGGATGACGTCAAGTCATCATGGCCCTTA

Both amplicons are 144 bp long with mismatches between the 2 sequences at positions 51, 67, 68, and 87.

Reaction product from each on-chip WGA reaction to be sequenced was first eluted off-chip and diluted into 30 or 40 μL of water. 5 μL of this was added to a 2^{nd} off-chip re-amplification reaction containing 31.25 μL amplification buffer, 37.75 μL water, and 1 μL amplification enzyme. All sequencing of WGA product was performed on an Illumina Genome Analyzer IIx protocol. E. coli samples were sequenced using 75 and 50 bp paired end reads for 1 and 2 rounds of WGA amplfication respectively. All oral sample data was sequenced using paired end reads.

Sequencing statistics are summarized in Table 1. Genome coverage >=1× for the single cell reactions ranges from 15.2% to 64.6% for the on-chip WGA product and from 24.5% to 62.77% after a second round of amplification, while the no-cell controls show no significant alignment to the reference genome. The approximately 1000 cell-reaction has comparable coverage to the single cell reaction with the highest coverage, indicating that the amplification is bias-limited and not template-limited.

TABLE 1.
Statistics for single E. coli sequencing.

| | On-chip WGA | | | | On-chip WGA + off-chip amplification | | | |
|---|---|---|---|---|---|---|---|---|
| | | % of reads | % of genome | % of genome | | % of reads | % of genome | % of genome |
| | # of 75 bp | aligned to | with > 1× | with > 10× | # of 50 bp | aligned to | with > 1× | with > 10× |
| | reads | genome | coverage | coverage | reads | genome | coverage | coverage |
| NTC 1 | 5663384 | 1.03 | 7.71 | 0.10 | 4639808 | 1.06 | 5.34 | 0.09 |
| NTC 2 | 4341480 | 0.25 | 8.74 | 0.05 | 6677002 | 0.09 | 5.45 | 0.02 |
| single cell 1 | 13483184 | 55.26 | 64.60 | 43.08 | 7458890 | 79.77 | 62.77 | 40.05 |
| single cell 2 | 9784130 | 47.22 | 40.57 | 22.51 | 6941568 | 80.17 | 41.87 | 22.92 |
| single cell 3 | 4708954 | 5.66 | 15.18 | 4.08 | 3304200 | 52.24 | 24.51 | 8.52 |
| single cell 4 | 5738682 | 1.04 | 18.58 | 1.56 | 7354470 | 68.39 | 42.30 | 24.84 |
| single cell 5 | 10268078 | 48.42 | 28.17 | 13.79 | 6085898 | 56.65 | 26.27 | 12.06 |
| single cell 6 | 10644760 | 34.51 | 30.31 | 13.99 | 5373842 | 42.56 | 27.46 | 11.96 |
| ~1000 cells | | | | | 8074402 | 78.78 | 61.60 | 34.53 |
| control gDNA | | | | | 62513866 | 90.90 | 99.78 | 99.69 |

1.10.4 Whole Genome Amplification of Microbial Aggregates Isolated from a Human Mouth.

To demonstrate the applicability of the device to the genetic analysis of microbes from environmental samples, WGA was performed on isolated single microbes from an oral biofilm sample resuspended in PBS and stained with a fluorescent DNA stain. WGA was performed on 70 sorted single cells as well as 5 no-cell control reactions. Twenty-two of the single cell reactions and one no-cell cell control reaction were randomly chosen for a second round of off-chip amplification and pyrosequencing. The sequencing data was then compared to a metagenome of the catalogued human oral microbiome (http://www.homd.org), suggesting that either multiple organisms were sorted into each storage element, or that contaminant DNA was present in the reactions. Nevertheless, this demonstration illustrates the use of the device and method for the genetic interrogation of microbes from environmental samples.

### 1.10.5 MDA-Based Whole Genome Amplification of Single Microbes

A commercially available MDA-based WGA protocol (Repli-G, Qiagen) was also evaluated using the same E. coli. strain. Initially, the protocol recommended by the manufacturer was followed, which lyses the cell and denatures the genomic DNA using an alkaline lysis buffer containing dithiothreitol (DTT), followed by addition of a neutralization buffer, and phi29 DNA polymerase and random primers for the MDA reaction. However, single-cell reactions were unsuccessful as determined by qPCR of a strain-specific fragment of the 16 s rRNA gene, as described above. Modifications to the recommended protocol were tested and it was discovered that the omission of DTT in the alkaline lysis buffer was critical for successful single-cell MDA.

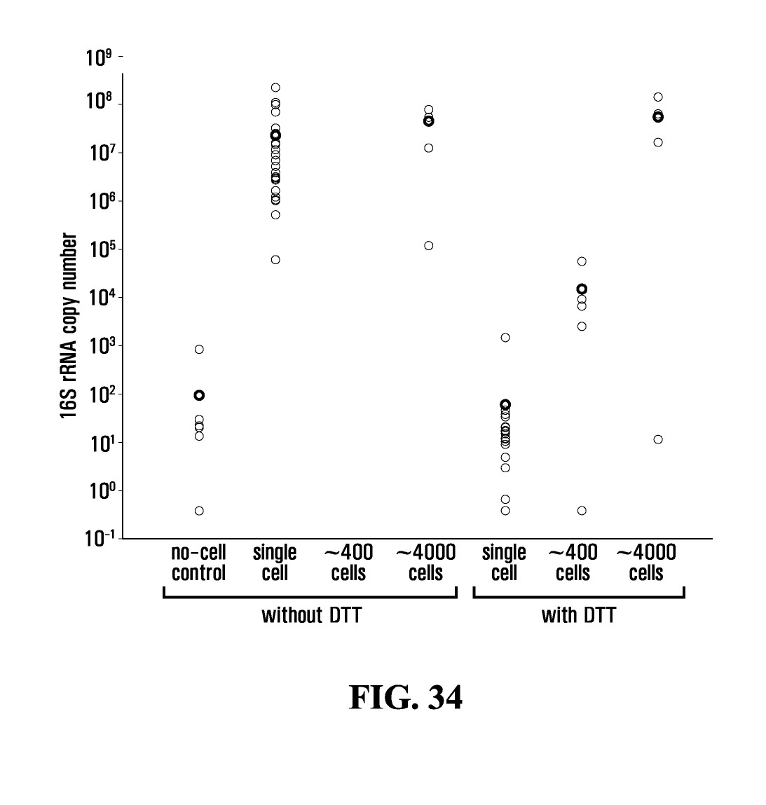

In order to directly compare reactions performed with and without DTT, a total of 90 reactions were performed on a single device using either a lysis buffer including DTT or another in which DTT was replaced with water. For each lysis buffer, MDA reactions were performed on single cells (N=30), ˜400 cells (N=5), and ˜4000 cells (N=5). Ten no-cell control reactions were also performed using the lysis buffer without DTT. After completion of the MDA reaction, products were eluted and amplified. 16S rRNA gene copy number in each reaction was quantified (FIG. 34).

MDA reactions using DTT in the lysis buffer resulted in variable 16S rRNA gene amplification dependent on the starting template quantity. Mean copy number yielded by the single-cell reactions was comparable to that of the no-cell control reactions (61 and 95 respectively), while the 400-cell and 4000-cell reactions yielded mean copy numbers of 1.5×10⁴ and 5.5×10⁷ respectively. In contrast, MDA reactions performed without DTT resulted in comparable amplified copy number for all starting template quantities with single-cell, 400-cell, and 4000-cell reactions having means of 2.2×10⁷, 6.9×10⁷, and 4.5×10⁷ respectively. As the DNA yield of MDA reactions should be independent of the amount of starting material, these results thus suggest that, in the present device, DTT has an inhibitory effect on the MDA reaction that is dependent on the starting template quantity.

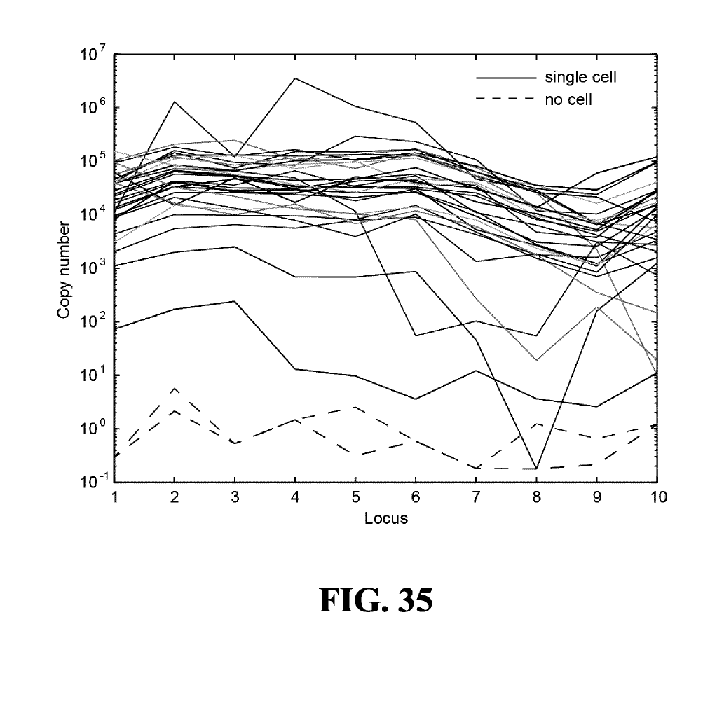

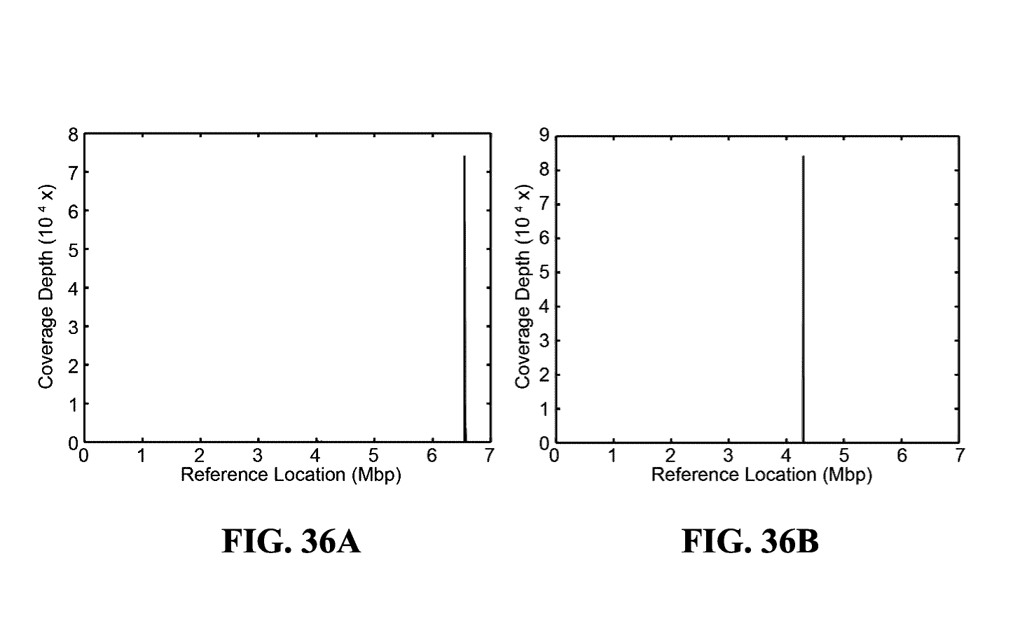

The mean 16S rRNA copy number resulting from single-cell MDA reactions performed without DTT was 8.8 times that of the single-cell PCR-based WGA reactions, and the coefficient of variation was 205%, 2.5 times less than that of the single-cell PCR-based WGA reactions. These results indicate that MDA may be a more robust protocol for single-cell WGA than the PCR-based protocol previously used. Accordingly, qPCR assays were used to quantify the amplified copy number of 10 single-copy loci across the E. coli genome in 2 no-cell control reactions and all 30 single-cell reactions performed without DTT in the lysis buffer (FIG. 35) as an initial gauge of representational bias in the MDA reactions. For all 30 single-cell reactions, the coefficient of variation of the mean copy number for all 10 loci was 84%, lower than what was reported in Marcy et al.

To more completely assess representational bias, sequencing of product from one of these reactions was performed, this time using an Ion Torrent PGM sequencing instrument. Conventional microlitre-volume MDA reactions were also performed and their products sequenced in order to compare their performance with microfluidic reactions. Sequencing was performed on a nanolitre-volume microfluidic single-cell reaction, a second microlitre-volume MDA reaction performed on the product of this microfluidic reaction, a microfluidic no-cell control reaction, a conventional microlitre-volume MDA reaction on a single FACS-sorted cell, and unamplified purified E. coli genomic DNA as a positive control. Sequencing reads and assembled contigs were aligned to the E. coli reference genome to generate coverage statistics for each sample, which are summarized in Table 2.

TABLE 2
Sequencing statistics for MDA-based WGA of single E. coli.

| | Unamplified | nL | nL/μL | μL | nL No-cell |
|---|---|---|---|---|---|
| | gDNA | MDA | MDA | MDA | MDA |
| Sequencing | 91.4 | 528 | 225 | 223 | 559 |
| effort (Mbp) | | | | | |
| Fraction of | 86.5% | 16.5% | 73.3% | 84.8% | 86.8% |
| data aligned | | | | | |
| to reference | | | | | |
| Fraction of | 99.5% | 99.4% | 99.4% | 99.0% | 3.28% |
| reference | | | | | |
| covered | | | | | |
| Mean length | 1.92 | 2.04 | 7.79 | 2.62 | 2.15 |
| of assembled | | | | | |
| contigs (kbp) | | | | | |
| Total length | 4.12 | 4.23 | 4.55 | 4.31 | 0.133 |
| of assembled | | | | | |
| contigs (Mbp) | | | | | |
| Fraction of | 85.6% | 87.6% | 94.6% | 87.4% | 2.73% |
| reference | | | | | |
| covered | | | | | |
| by contigs | | | | | |

As can be seen in Table 2, relative to the other sequenced samples, the nanolitre-volume MDA product contained a very small fraction of sequencing data that aligned to the expected reference genome, suggesting that some contamination was present.

### 1.10.6 Exceptionally Low Representational Bias in Nanolitre MDA

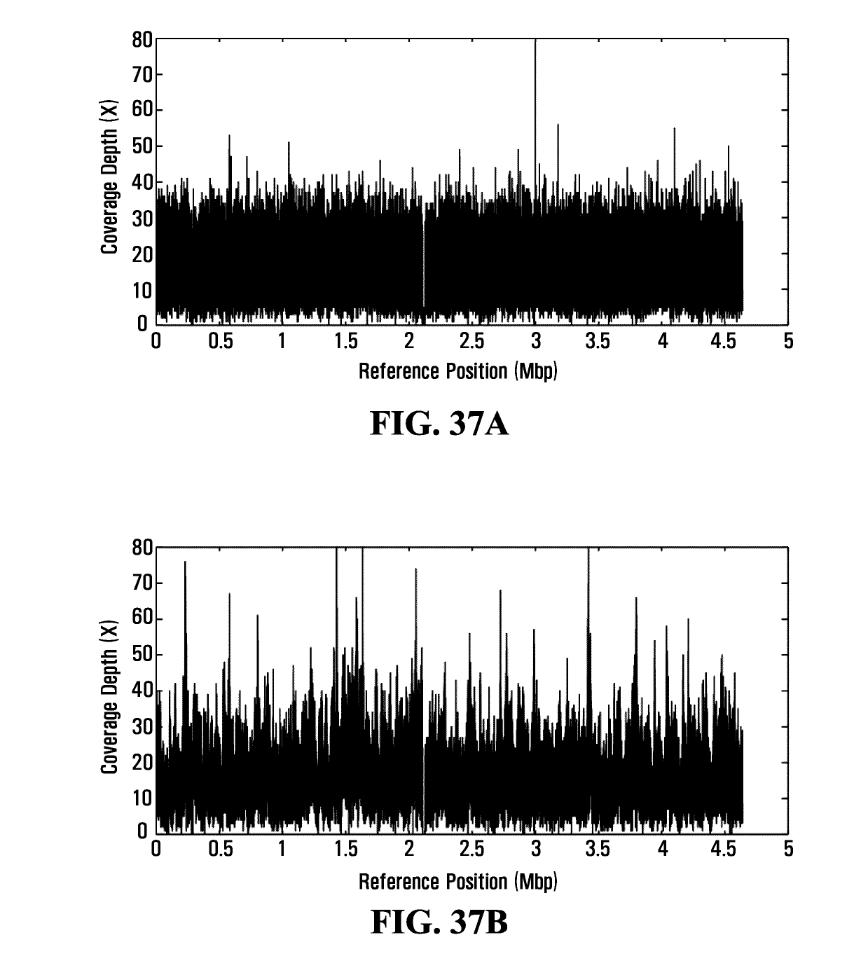

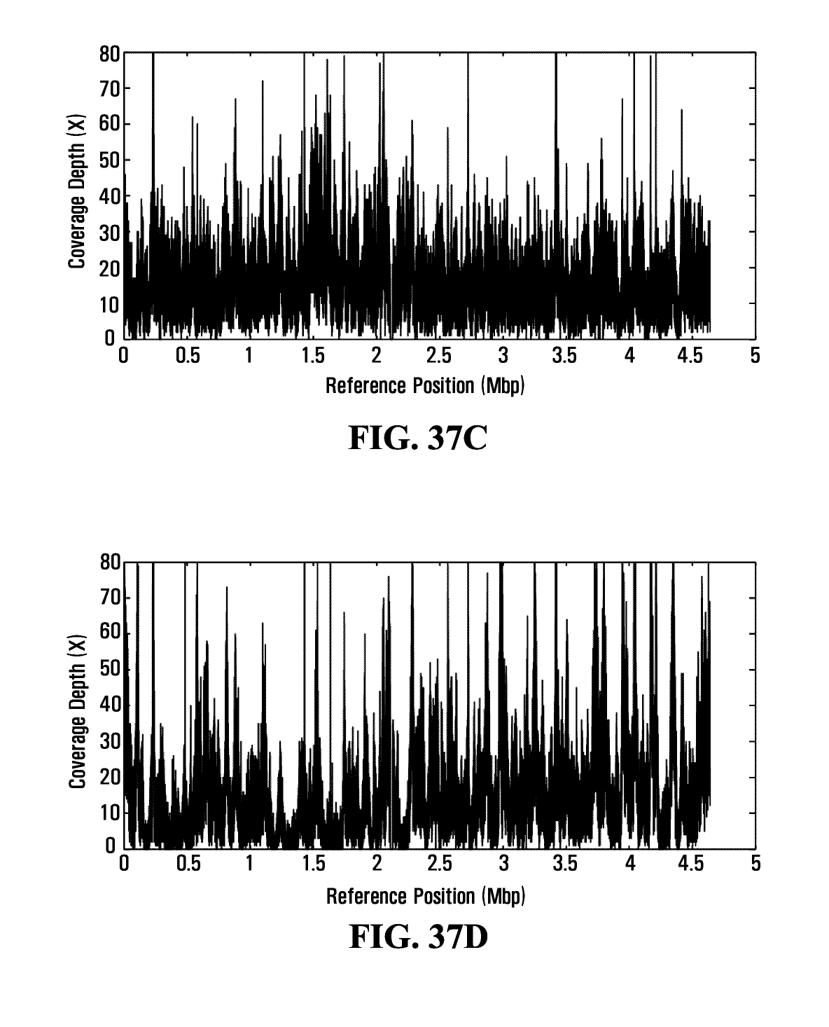

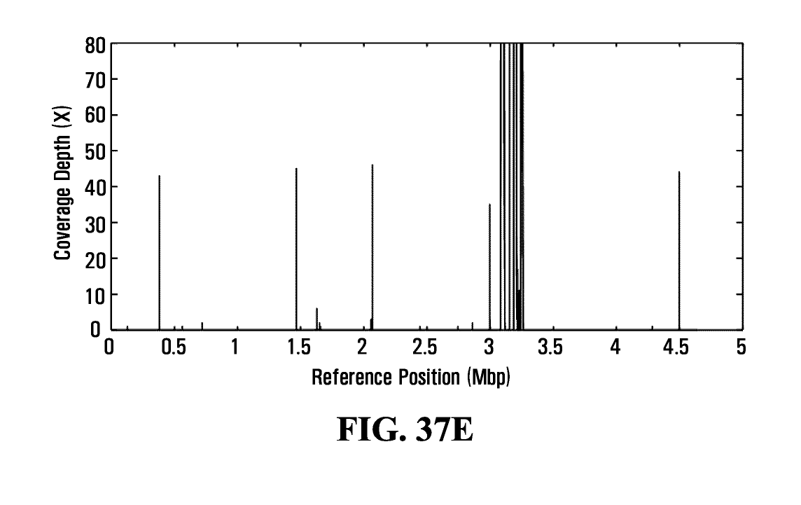

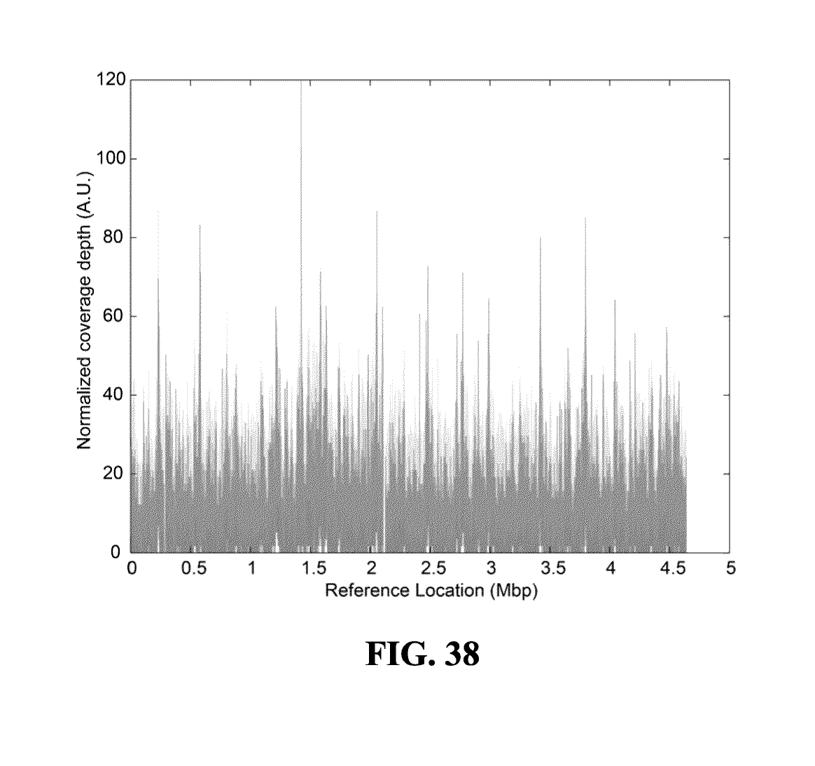

To compare the representational bias of all MDA reactions, the sequencing data aligned to E. coli from each reaction type was first randomly subsampled at mean coverage depths ranging from 1× to 16× in order to compare equal quantities of data for each reaction. Coverage maps for each reaction type displaying the number of sequencing reads covering each position of the E. coli reference genome at 16× mean coverage depth are shown in FIG. 37. From these coverage maps, it can be qualitatively seen that of the single-cell MDA reactions, the nanolitre-volume reaction has the least variation in coverage followed by the combined nanolitre/microlitre reaction and the microlitre reaction in order of increasing variation. Overlaid normalized coverage maps, showing minimum to maximum coverage, for the two nanolitre single-cell reactions sequenced are shown in FIG. 38.

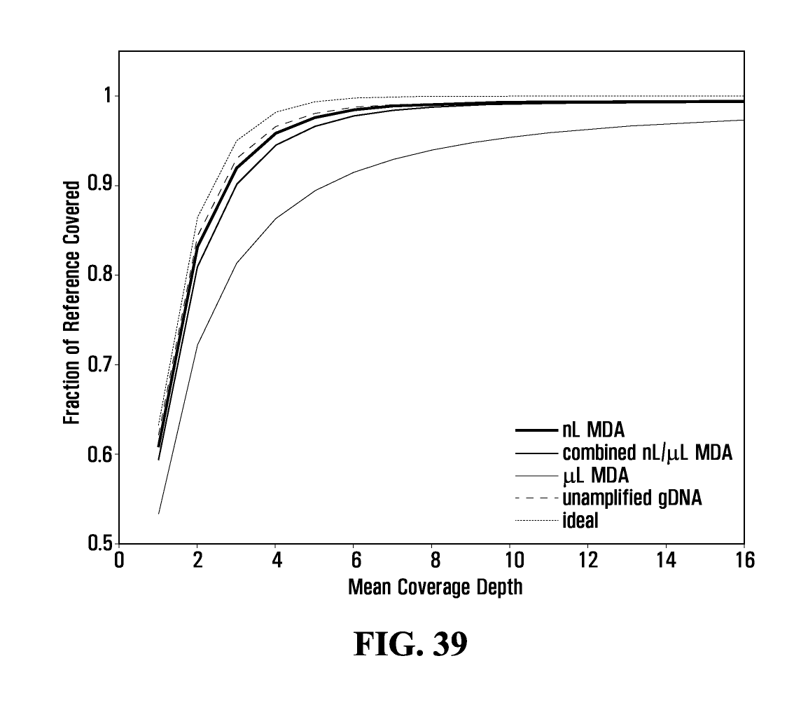

To more quantitatively assess the bias of each MDA reaction type, the fraction of the reference covered at mean coverage depths ranging from 1× to 16× were found as shown in FIG. 39. The ideal coverage that would be obtained from a perfectly unbiased sample, as predicted by Poisson statistics, is also shown. The single-cell nanolitre and combined nanolitre/microlitre reactions have very similar reference coverage to that of the unamplified genomic DNA at all mean coverage depths, while that of the single-cell microlitre reaction is significantly lower, confirming that bias is greatly reduced by performing MDA in a nanolitre volume. The nanolitre reaction in fact has slightly higher reference coverage than that of the combined nanolitre/microlitre reaction for all mean coverage depths, suggesting that a microfluidic reaction alone can achieve equivalent or slightly reduced representational bias relative to the combined nanolitre/microlitre reaction with a thousand times lower reagent consumption. As might be expected, this difference is most pronounced at lower mean coverage depths and decreases at higher depths. At a mean coverage depth of 2×, the unamplified genomic DNA, nanolitre reaction, combined nanolitre/microlitre reaction, and microlitre reaction have reference coverage of 84.4%, 83.1%, 81%, and 72.2% respectively. At a mean coverage depth of 8×, the single-cell nanolitre reaction covers 99.1% of the reference. These results, to the inventor's knowledge, represent the highest reference coverage and lowest representational bias obtained from a single-cell WGA reaction reported to date.

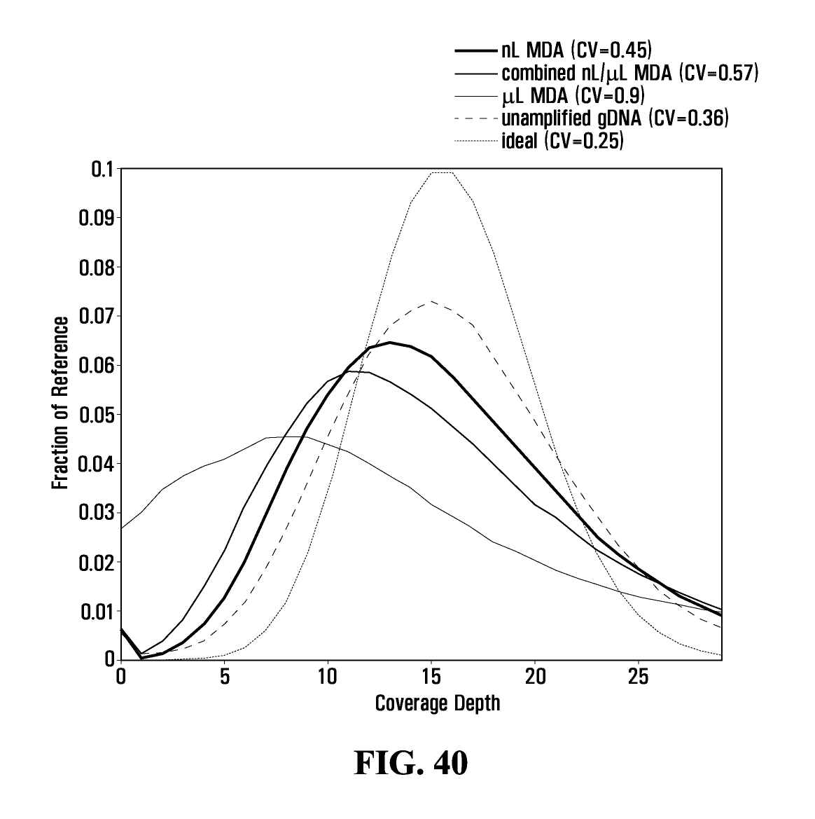

To further depict the bias of each reaction type, the fraction of the reference genome covered at various depths for a mean coverage depth of 16× was plotted in a histogram (FIG. 40). The result that would be obtained for an ideally unbiased sample, as predicted by Poisson statistics, is also shown. A numerical measure of representational bias is the coefficient of variation (CV) of the coverage of each position of the reference. CV values for an ideal sample, the unamplified genomic DNA, the nanolitre single-cell MDA reaction, the combined nanolitre/microlitre single-cell MDA reaction, and the microlitre single-cell MDA reaction are 25%, 36%, 45%, 57%, and 90% respectively, again illustrating that the nanolitre MDA reaction has the lowest representational bias of the three single-cell reactions.

The demonstrated ability to perform single-cell WGA at high throughput with the lowest reported representational bias to date using nanolitres of reagent per reaction has significant implications for future single-cell genomic studies. Besides the obvious reduction in WGA reagent costs, reduced representational bias allows for genome coverage with reduced sequencing effort, thus also reducing sequencing costs. This capability thus has the potential to enable currently intractable genomic studies of large numbers of single cells.

### 1.9.7 Environmental Genomics

PCR-based WGA chemistry was applied to the WGA and sequencing of microbes in environmental samples to explore genomic relationships within natural microbial communities. Samples were selected from three environments representing varying levels of structural complexity. Environment 1 (ENV1) was a bacterial enrichment culture from seawater chosen to represent a low-complexity environment. Environment 2 (ENV2) was a human oral biofilm chosen to represent a high-complexity microenvironment. Environment 3 (ENV3) was a 3-8 μm fraction from deep-sea sediments associated with methane seepage. Based on the complexity and aggregation state of each environment, alternative on-chip sorting strategies were used. Single cells were isolated from ENV1, individual extended filamentous aggregates were isolated from ENV2, and individual spherical aggregates were isolated from ENV3. A total of 203 on-chip WGA reactions using the previously described PCR-based protocol were performed (50 in ENV1, 60 in ENV2, 93 in ENV3) including 5 no-cell controls consisting of equal volumes of cell suspension fluid containing no visible cells.

A total of 74 samples representing each of the environments were randomly selected for a subsequent round of off-chip amplification in a microlitre-volume and sequencing library construction, resulting in 72 successful libraries: 24 single cells from ENV1, 22 filamentous aggregates from ENV2, 23 spherical aggregates from ENV3, and 3 no cell control samples. The two remaining samples were excluded due to suspected contamination or mislabeling during library preparation. Samples were indexed, pooled and sequenced on a single lane of an Illumina Genome Analyzer II instrument, generating a total of 4.8 billion bases in 64 million reads. Assemblies were performed for each sample and contigs greater than 200 bp in length were used for further analysis. The number of contigs for each sample varied between environments with ENV1 assemblies yielding the highest average number per sample (mean of 1,998 contigs covering 70% of reads), followed by ENV2 (mean of 659 covering 76% of reads) and ENV3 (mean of 431 contigs covering 70% of reads). This correlated with contig length differences between samples with mean contig lengths of 471, 424, and 324 bp for ENV1, ENV2, and ENV3 respectively. Individual assemblies were limited by sequencing depth and that the higher number of contigs in ENV1 is likely due to reduced sample complexity. No-cell controls resulted in 7-20 contigs per sample, which covered less than 30% of reads.

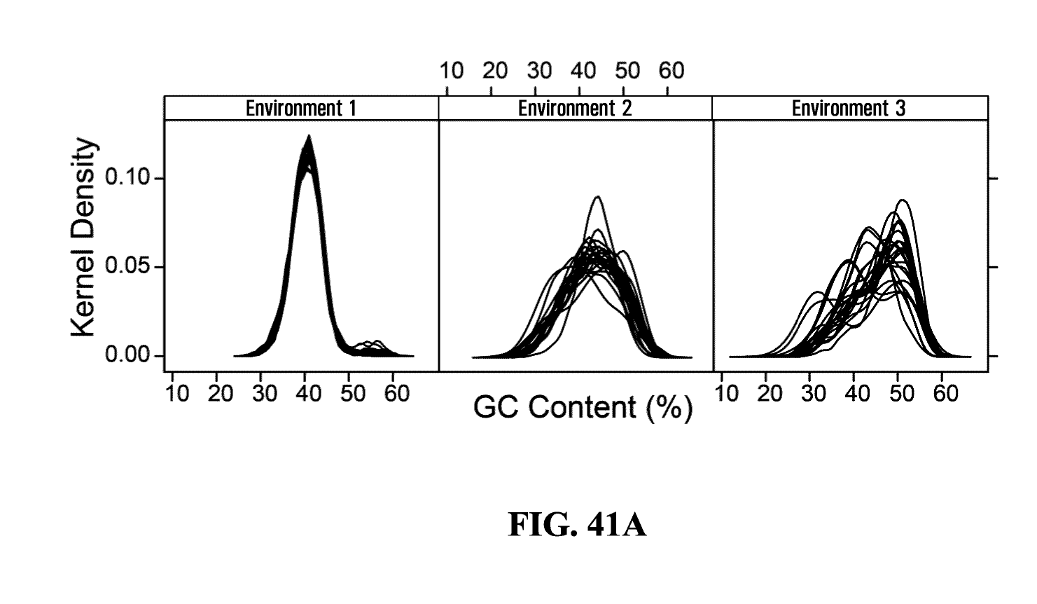

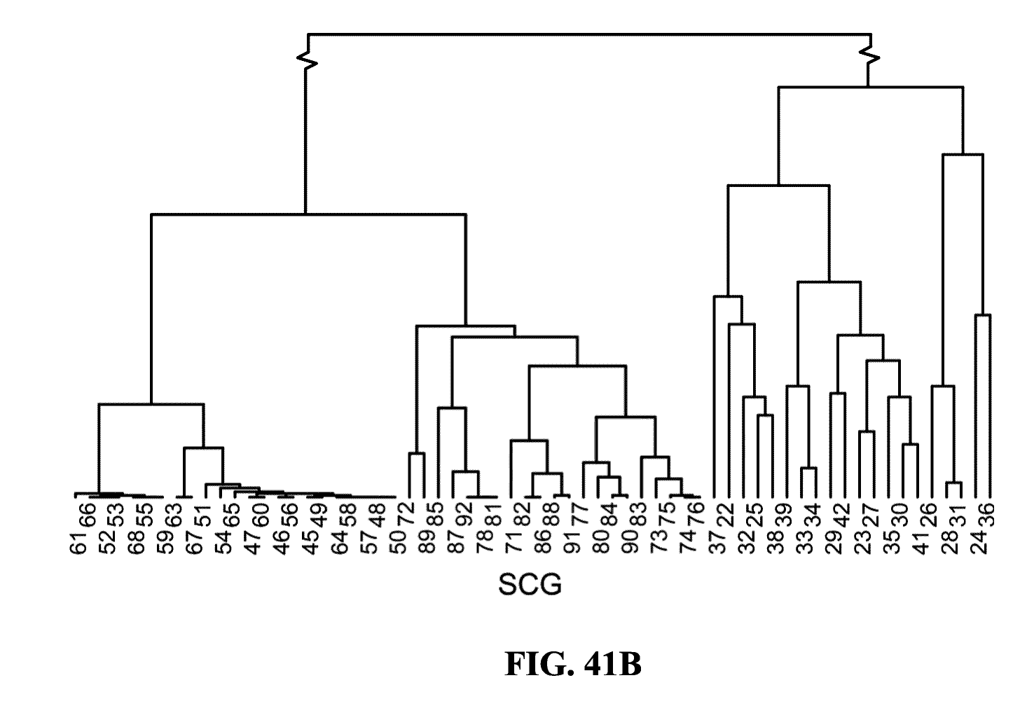

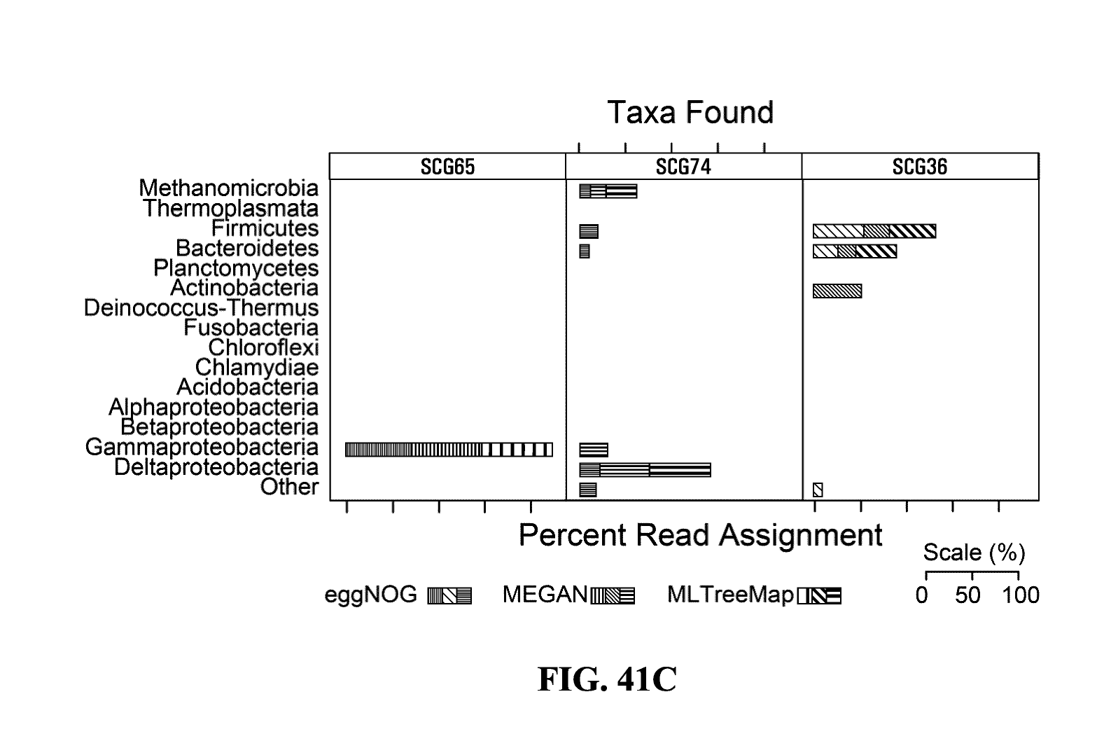

The genomic complexity of the indexed samples was first analyzed by plotting kernal density functions of GC composition. All ENV1 samples exhibited a single characteristic peak, consistent with targeted amplification of closely related donor genotypes (41A). By comparison, the GC content exhibited by ENV2 samples was a mixture of unimodal and multimodal curves consistent with targeted amplification of both single-cell genomes and mixtures of adhering cells (FIG. 41A). Finally, ENV3 samples also exhibited multimodal curves and single spreading peaks consistent with amplification of multicellular aggregates (FIG. 41A). The taxonomic structure of each sample was then determined using a tripartite binning approach. A stringent binning criteria was initially adopted based on 40 conserved phylogenomic markers mapped onto the tree of life using MLTreeMap. However, due to low sequencing depth only a handful of these markers were identified. To increase taxonomic resolution, the eggNOG and NCBI ref_seq databases were queried using open reading frames (ORFs) predicted on contigs from each indexed sample. Results from the ref_seq search were then mapped onto the NCBI taxonomic hierarchy using Metagenome Analyzer (MEGAN) to define the most probable ancestor for each query sequence. Open reading frames assigned to taxonomic nodes by MEGAN were normalized by the fraction within each sample and hierarchically clustered, resulting in three distinct clusters for the ENV1, ENV2 and ENV3 samples. Branch lengths within each of the three clusters were consistent with increasing levels of genomic complexity with ENV1 samples exhibiting the least complexity followed by ENV3 and ENV2 (FIG. 41B).

The taxonomic origins of ORFs predicted in ENV1 samples were primarily affiliated with the genus Pseudoalteromonas within the Gammaproteobacteria. Based on hierarchical clustering results two genotypic variants were resolved, consistent with the presence of closely related subpopulations within the enrichment culture. ORFs from ENV2 samples were dominated by known human oral microbiome constituents including Capnocytophaga and Flavobacterium within the Bacteroidetes, Corynebacterium, Rothia, Kocuria and Actinomyces within the Actinobacteria, Fusobacterium within the Fusobacteria, and Clostridium and Streptococcus within the Firmicutes (FIG. 41C). Low-level representation of the candidate division TM7 was also observed. Different samples contained overlapping but not identical subsets of these taxonomic groups, with Streptococcus, Corynebacterium and Capnocytophaga being the most common overlapping taxa. Many of the taxonomic configurations observed in ENV2 samples have been previously described in the context of coaggregation and biofilm formation within the oral cavity (135-138), and several have been directly visualized using combinatorial labeling and spectral imaging techniques (139). ORFs from ENV3 samples were dominated by sulfate reducing bacteria (SRB) affiliated with Desulfatibacillum, Desulfobacterium and Desulfococcus within the Deltaproteobacteria. Intermediate levels of representation were observed for unaffiliated Gammaproteobacteria, and Betaproteobacteria in addition to methanogenicarchaea. Low-level representation of other taxa was observed in specific ENV3 samples, including ORFs affiliated with Alphaproteobacteria, Bacteroidetes, Firmicutes, Chloroflexi and Clostridia.

Here, it has been demonstrated how phenotype-based sorting and droplet-based WGA followed by sequencing can be used to identify single microbes and members of microbial aggregates with a particular morphology. In the latter case, this ideally allows for the inspection of entire genomes of constituent members within aggregates, going beyond mere co-localization of small numbers of genes (5, 16), and enabling the analysis of metabolic pathways that can more precisely characterize potential symbiotic relationships within physical aggregates.

### i) PCR-Based Genotyping of Single Human Tumour Cell Nuclei

Cellular heterogeneity is increasingly being shown to be a characteristic of human disease that has implications for both diagnosis and treatment. For example, in cancer, genetic analysis of different spatial regions within individual tumours have revealed branching patterns of tumour “evolution”, resulting indistinct subpopulations that can be grouped based on genetic aberrations such as genomic loci copy number variation, allelic imbalance, and mutations that are putative disease “drivers”. This implies that specimens obtained from single biopsies may only reveal a subset of the aberrations of the whole tumour and may not identify those that are ubiquitous and thus important targets for therapy. It has also been shown that genetic clonal diversity can predict progression from a premalignant condition to a cancer, suggesting that increased diversity provides a wider base upon which natural selection can act to produce a tumour.

In order to study cancer progression at higher spatial resolution, clonality can be analyzed at the single-cell level. Fluorescent in-situ hybridization (FISH) has been used to enumerate copy number variations of 8 genetic loci in individual cells, enabling inference of evolutionary trees based on the frequencies of these variations. PCR-based WGA and sequencing of FACS-sorted single-cell tumour nuclei has been used to analyze loci copy number variation across the entire genome in 200 cells, derived from two separate cancers, to determine that the tumours progressed in “punctuated” clonal expansions that yielded distinct tumour subpopulations each distant from their root. Similarly, MDA-based WGA and sequencing of 25 single cells from a single tumour, isolated by manual micromanipulation, indicated the tumour likely did not result from mutations typical of that cancer and that, in contrast to the above study, there were no distinct clonal subpopulations.

Clonal frequencies of somatic mutations in breast cancers have been estimated by sequencing PCR amplicons from bulk DNA derived from tumours. In order to more exactly determine the distribution of mutations within a tumour, however, the loci of interest must be amplified and sequenced in single tumour cells. This can be accomplished in the present microfluidic device by single-cell PCR-based genotyping as demonstrated herein on single bacteria.

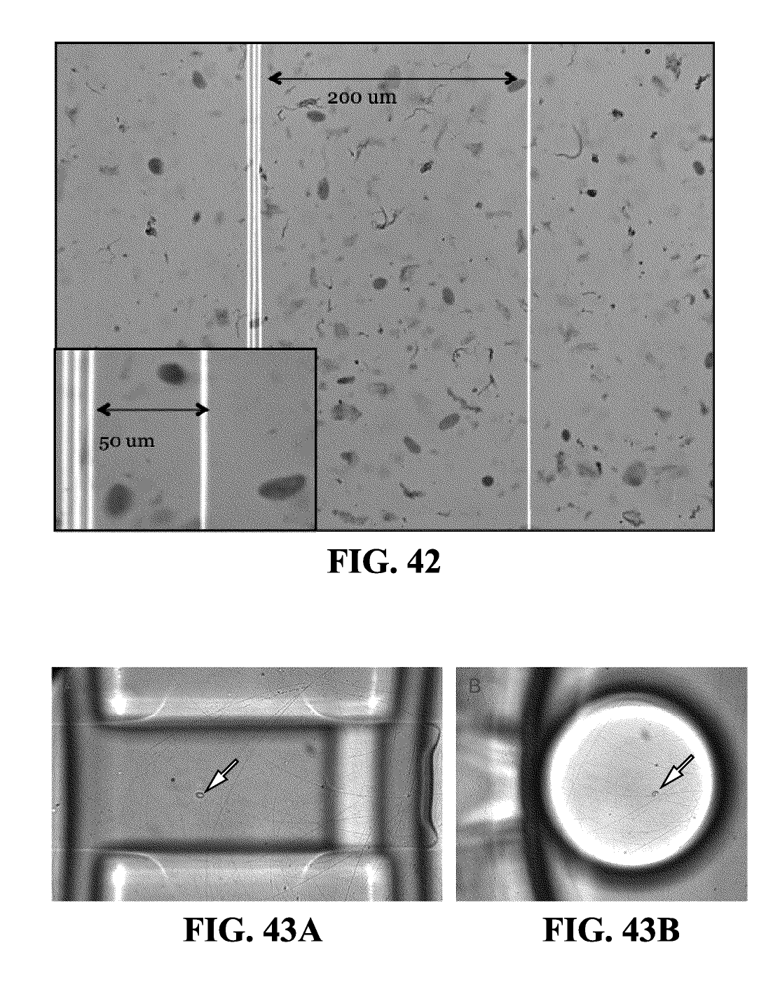

As it is difficult to derive single-cell suspensions from solid tumour tissue, cell nuclei can be extracted from the tumour samples. However, the resulting nuclei samples are highly heterogeneous in morphology, as the nuclei themselves can vary in size and the extraction process leaves a variety of cell debris in the sample (FIG. 42). The ability to perform morphology-based sorting of nuclei using the present device is thus a significant advantage.

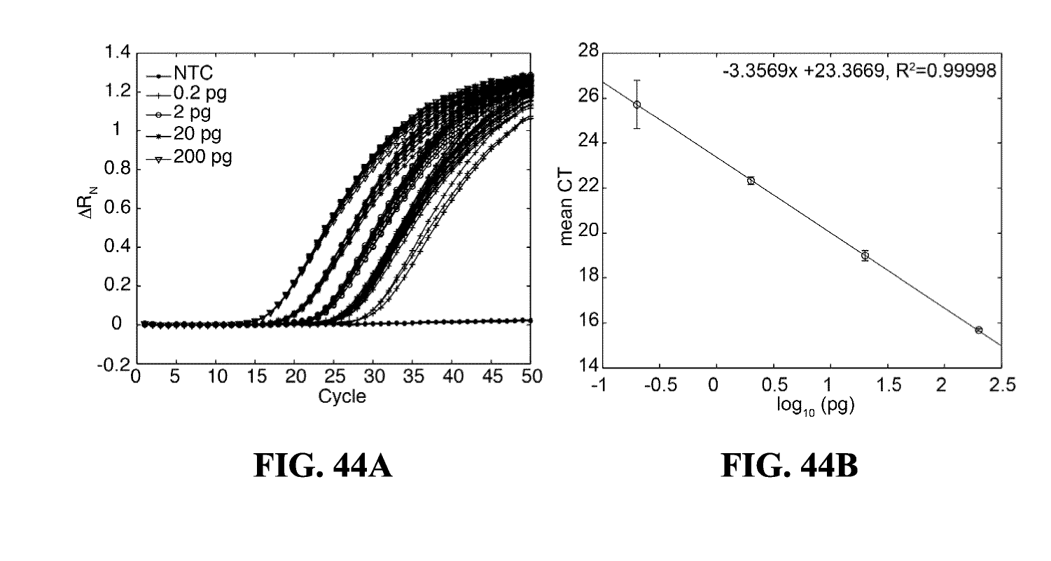

As a first stringent test of genomic PCR on primary breast cancer pleural effusion cell nuclei, on-chip qPCR targeting the RNase P gene present at one copy per haploid genome (2 per cell), was performed on single nuclei (N=80), ˜50 haploid genome copies of purified human gDNA (N=5), and suspension fluid containing no nuclei as determined by microscopy-based sorting (N=5). Micrographs of a cell nucleus in the cell-sorting module and in a stored droplet are shown in FIG. 43A and FIG. 43B, respectively. The 3-minute PCR hot start at 95 C. was used to lyse the nuclei. qPCR curves indicated that the target sequence was successfully amplified in 78 of 80 (98%) single nuclei, all 5 gDNA reactions, and 2 of 5 no-nuclei control reactions (FIG. 44A). The latter result is most likely due to free gDNA from the nuclei sample in the suspension fluid. The difference in mean CT between reactions containing single nuclei (containing 2 gene copies) and 50 haploid genome copies (FIG. 44B) was found to be 4.59 cycles, indicating an assay efficiency of 101.6%. This nearly ideal assay efficiency indicates that the gDNA within the nuclei is made accessible to PCR reagents by the protocol used, and, as in the PCR experiments on single bacteria, show that with an optimized assay and efficient lysis, robust PCR amplification can be achieved from single human cell nuclei.

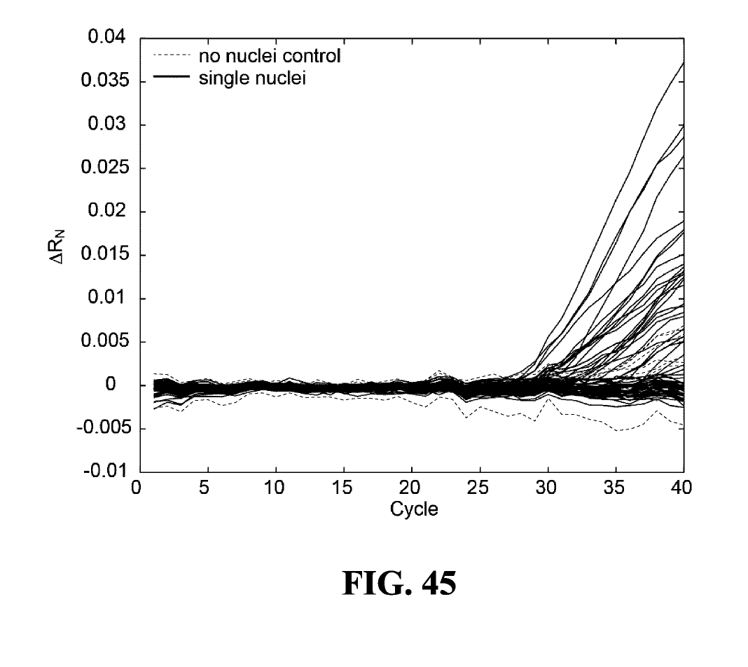

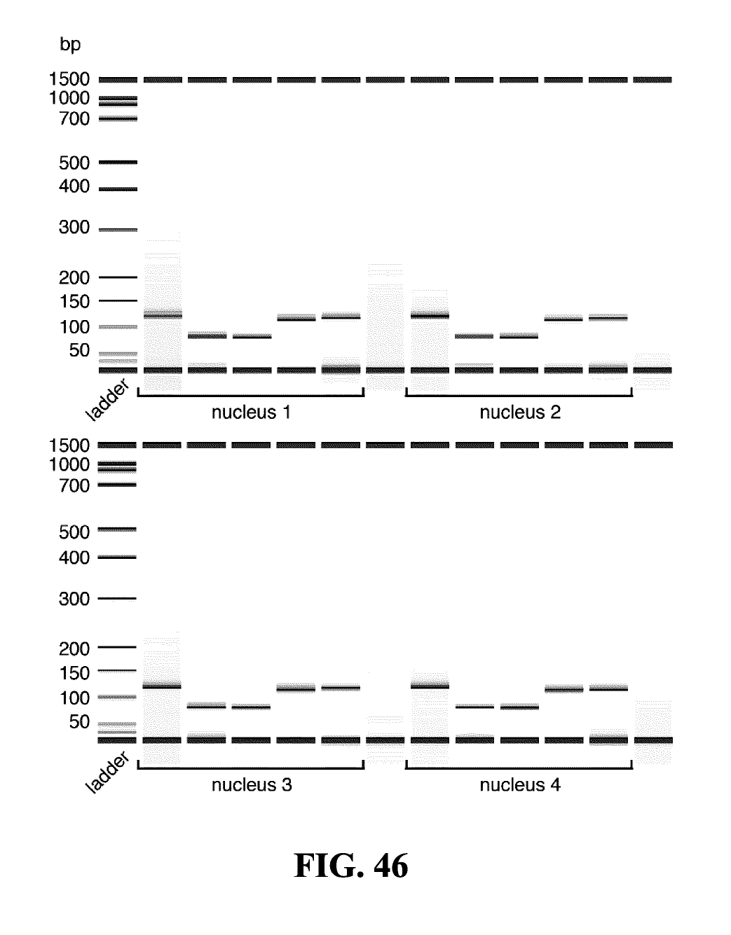

Having established that the gDNA of cell nuclei could be accessed for on-chip PCR, primer pairs targeting 6 genomic loci were then tested in multiplex qPCR reactions, including an intercalating dye for real time reaction monitoring, on single nuclei (N=63) and no-nuclei controls (N=10). Five of the 6 loci contain somatic mutations of interest: FGA, GOLGA4, KIAA1468, KIF1C, and MORC1 and the sixth locus was a multi-copy germline control NOTCH2NL. qPCR curves for all reactions are shown in FIG. 45. While these qPCR curves provide some indication of amplification, it should be noted that they are less indicative of reaction progress than qPCR curves in single-plex PCR reactions due to interaction between primer pairs for different assays. CT values were observed to be quite late for all single-nuclei reactions. Nevertheless, these values were used as a guide to select a subset of single-nuclei reactions for further analysis. Following on-chip 6-plex PCR, reaction products were eluted and off-chip single-plex PCR of each of the 5 somatic mutation loci was performed on the product of 7 of the on-chip single nuclei reactions with the lowest on-chip qPCR CT valuesas well as 2 of the no-nuclei control reactions. Each amplicon of these single-plex PCR reactions were then visualized by capillary electrophoresis. Plots for 4 of the single nuclei reactions and both no-nuclei control reactions are shown FIG. 46. In total, 33 of 35 (94%) possible amplicons from the 7 single-nuclei reactions analyzed were successfully amplified as determined by the presence of a band with expected size. Bands were also seen for 3 amplicons in the no-nuclei controls.

All 45 single-plex PCR amplicons (7 single nuclei and 2 no-nuclei controls with 5 loci each) were further analyzed by sequencing on an Ion Torrent PGM instrument. The amplicons from each nucleus and all amplicons from both no-nuclei controls were pooled and barcoded for sequencing. For comparison, 20 ng of bulk gDNA extracted from millions of cells was also subjected to the same protocol of multiplex PCR followed by single-plex PCR, but in conventional microlitre-volumes at a template concentration approximately 10 times greater than in on-chip single-nuclei reactions. Amplicon sequencing data binned by chromosome coverage from a representative single nucleus and bulk gDNA indicates that the on-chip multiplex PCR amplifies target loci with similar representational bias to the microlitre-scale reaction performed on bulk gDNA. The higher coverage of chromosome 3 is due to the fact that two of the loci are located on that chromosome. The number of reads obtained from each amplicon, the fraction of reads matching mutations reported in the literature, the means and coefficients of variation of these statistics for all single nuclei, and the mutational frequencies obtained from analysis of bulk DNA in are not shown.

The mutational frequencies observed in single nuclei are relatively variable for all loci, with coefficients of variation above 0.4 with the exception of MORC1, suggesting a heterogeneous population. For the most part, frequencies are close to the expected theoretical ratios of 0, 0.5, and 1, corresponding to nuclei that are homozygous for a non-mutant variant, heterozygous, and homozygous for the mutation respectively. Departures from these ratios can possibly be explained by loci copy number variations that result more than 2 alleles.

This application illustrates how the single-cell genomic analyses, previously demonstrated on microbes, are equally applicable to eukaryotic cells. This work will allow for the exact determination of the clonal frequency of mutations in an unprecedented number of single cancer cells, which will ultimately enable the examination of clonal evolution with unparalleled resolution and scale.

### ii) Single-Cell Whole Transcriptome Amplification

While genetic aberrations typical of diseases such as cancer are sources of cellular heterogeneity, even cells of a healthy organism, which essentially share the same genome, clearly exhibit phenotypic diversity that allows for a plethora of physiological functions. These differences are due to cell-to-cell variations in the transcriptome, the set of all RNA molecules that comprises the functional output of the genome. It is generally thought that persistent variation in genetically identical cells is caused by the stochastic nature of gene expression, due to small copy numbers of genes, and the presence of multiple metastable transcriptional states. In order to fully understand the transcriptional mechanisms responsible for this cell-to-cell heterogeneity, vital to the determination of cell fate, or to identify minority cell populations based on transcriptional state, it is necessary to analyze the transcriptomes of single cells. The combination of new methods for the amplification of RNA quantities present in a single cell and the high throughput of modern sequencing instruments now offers the possibility of sequencing the entire transcriptome (RNA-seq) of many single cells. Importantly, sequencing of the transcriptome allows for the identification and discovery of post-transcriptional modifications to RNA molecules that may alter proteins coded by the genome, which may play a role in disease.

As in whole genome amplification, the minimization of representational bias is crucial in whole transcriptome amplification (WTA) for RNA-seqin order to both minimize sequencing effort and allow for accurate measurement of the relative abundances of RNA molecules. In addition to the obvious advantage of lowered reagent costs, single-cell WTA in nanolitre-volumes may benefit from lowered representational bias relative to microlitre-volumes, as has been previously shown herein with single-cell multiple displacement amplification.

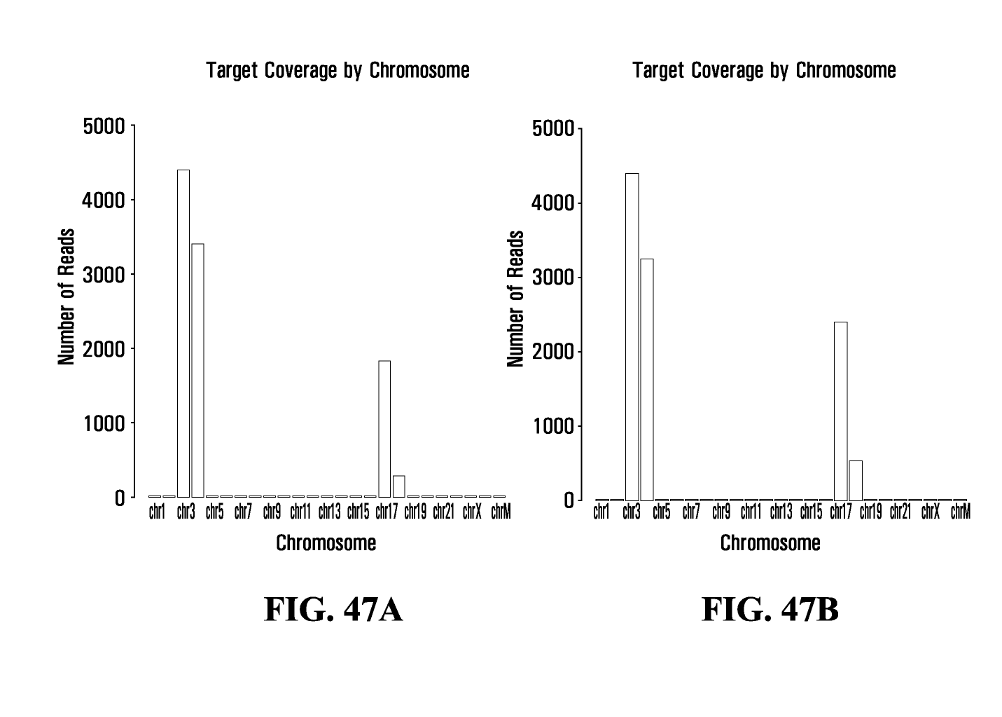

As a first test of RNA quantification, quantitative reverse-transcription PCR (qRT-PCR) targeting GAPDH mRNA was performed on purified RNA derived from a human k562 cell line. qRT-PCR reactions were assembled in stored droplets containing 0.2 pg (N=32), 2 pg (N=15), 20 pg(N=9), 200 pg(N=9), and 0 pg (N=4) of purified RNA and RT-PCR master mix including primers and a hydrolysis probe for the detection of the GAPDH gene. The amount of total RNA present in a single mammalian cell is ˜20 pg. Following reaction assembly, on-chip qRT-PCR was performed as previously described for 50 cycles, with fluorescent images of the droplet array being acquired at each cycle. Fluorescent images were analyzed using custom software in order to generate a qPCR curve for each stored droplet (FIG. 47A) and CT values were extracted from these curves (FIG. 47B). The PCR efficiency, determined by the slope of the fitted line through the CT vs. log₁₀ (template quantity) data points, is 98.6%, indicating that quantitative measurements of RNA abundance can be performed in the device.

Next, a commercially available WTA protocol (Omniplex, Sigma) was tested on purified RNA. The multistep protocol consists of priming of all RNA by primers composed of random hexamers and a universal sequence, followed by reverse transcription which produces a library of cDNA fragments flanked by the universal sequence, and PCR amplification of the fragment library using universal primers. The protocol was tested on 0.2 pg (N=10), 2 pg (N=10), 20 pg (N=10), 200 pg (N=10), and 0 pg (N=5) of purified RNA derived from k562 cells in order to span the range of RNA quantities expected in a single mammalian cell. Following on-chip WTA, reaction products from all stored droplets were eluted and cDNA abundances were analyzed by qPCR.

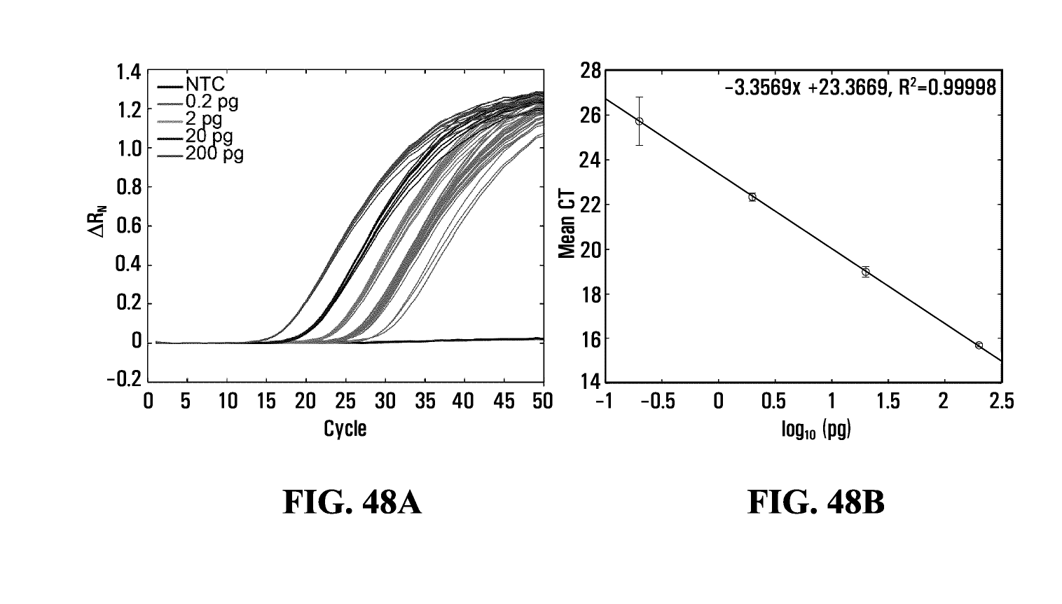

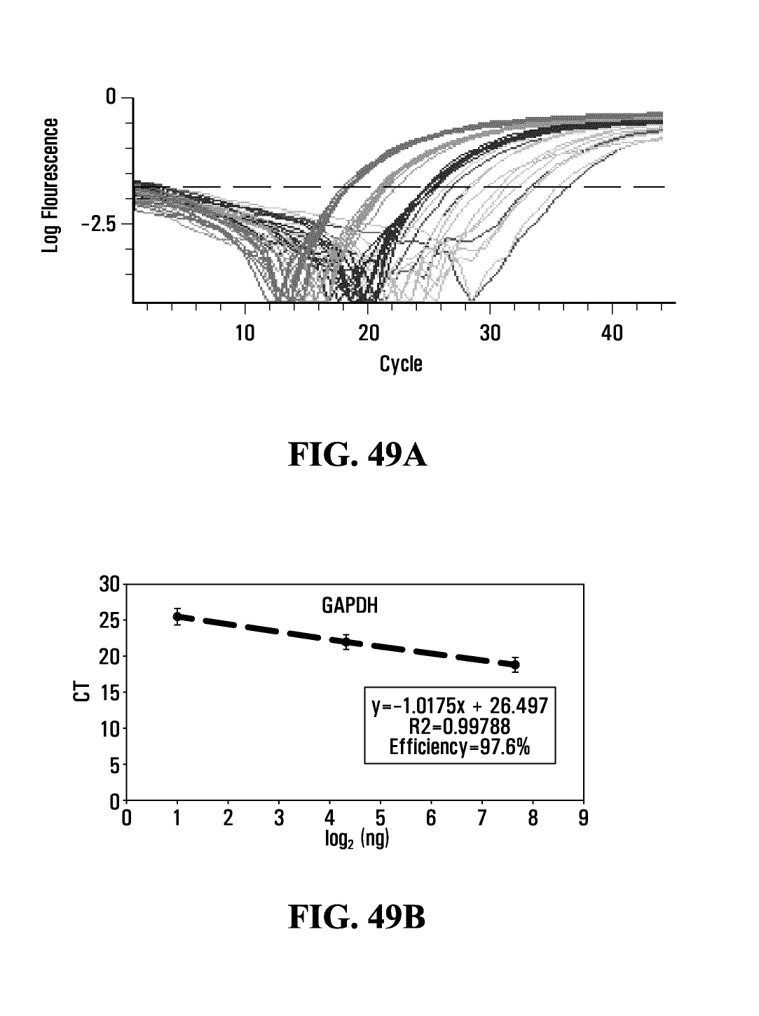

GAPDH cDNA, a common reference gene, was quantified by conventional microlitre-volume qPCR in the WTA product. qPCR curves on WTA product from each starting RNA quantity are shown in FIG. 48A. qPCR curves for the 2, 20, and 200 pg WTA reaction products are tightly clustered, whereas those of the 0.2 pg WTA reaction products have large spread and have CT values comparable to those of the NTC WTA reactions. WTA on 0.2 pg of RNA was thus considered unreliable. Mean CT values and standard deviation of the 2, 20, and 200 pg WTA reaction products are plotted in FIG. 48B. The exceptionally low standard deviation for all starting RNA quantities highlights the high reproducibility of both the on-chip WTA reactions and the elution process. The amplification efficiency, determined by the slope of the fitted line through CT vs. log₂ (template quantity) data points, is 97.6%, indicating highly quantitative on-chip WTA on RNA quantities spanning an order of magnitude above and below that expected in a single mammalian cell.

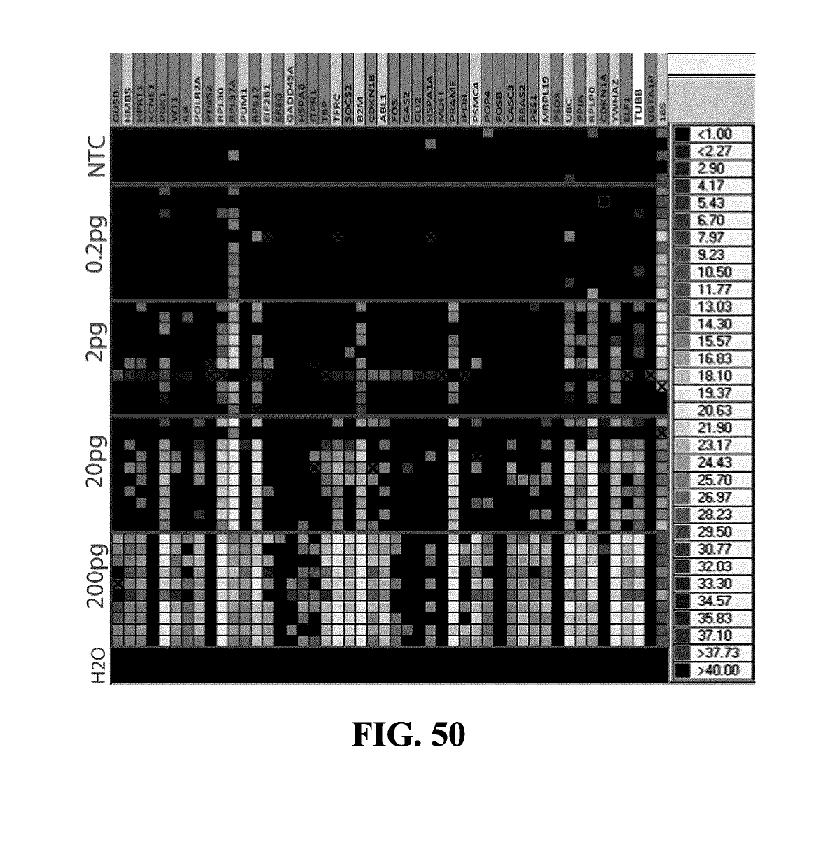

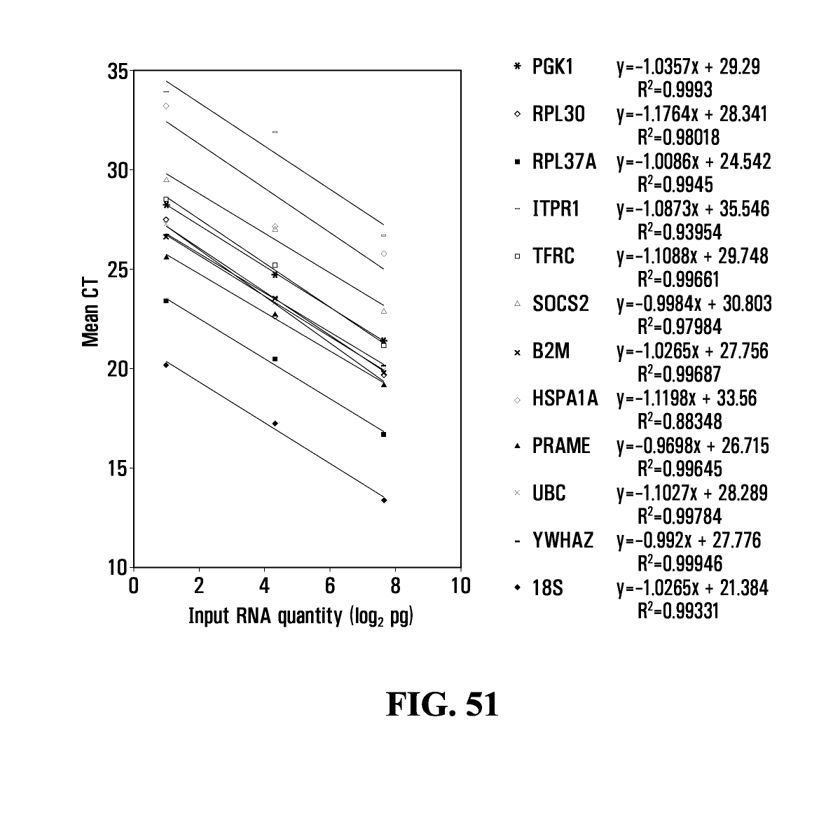

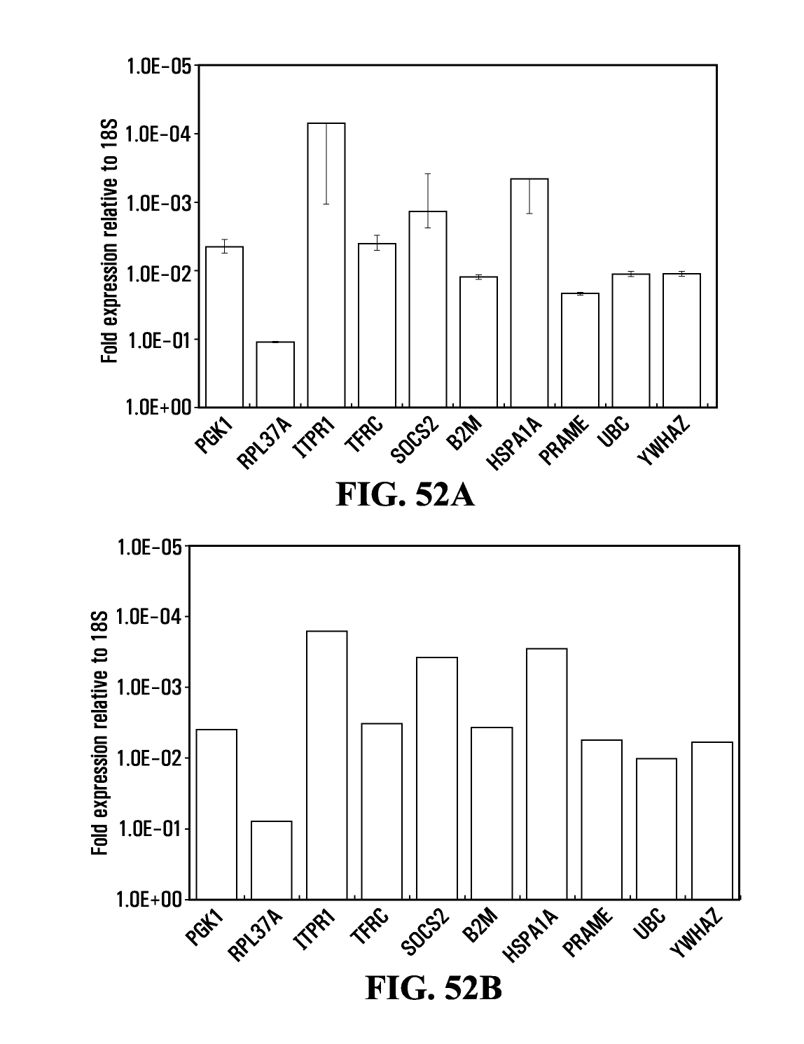

In order to further assess WTA performance, a panel of 48 qPCR assays targeting endogenous control genes was applied to WTA product from each on-chip reaction using a commercial microfluidic qPCR device (48.48 Dynamic Array, Fluidigm) that allows for the application of up to 48 assays against 48 samples in nanolitre-volume reactions. A heat map depicting CT values for all reactions is shown in FIG. 50. For each gene, CT values for were plotted against log₂ (template quantity) and PCR efficiencies were calculated. These plots are shown in FIG. 51 for genes which exhibited PCR efficiencies between 85% and 115%. Based on these results, it can be concluded that on-chip WTA is quantitative for at least the genes shown in FIG. 51. To compare the WTA performance in microfluidic and conventional formats, WTA was also performed on 100 ng of purified RNA in microlitre-volume reactions and products were again quantified by qPCR in a Dynamic Array device. Abundances of the above 10 genes relative to the 18S rRNA gene, which was found to have the lowest CT of all genes quantified, were determined by comparison of mean CT values for all starting RNA quantities and calculated as 2^{ΔCT} in both on-chip and conventional WTA reaction products (FIG. 52). The similarity of gene abundances in the microfluidic and conventional formats offers further evidence that the on-chip WTA protocol is quantitative.

The flexibility of programmable droplet-based reaction assembly can be exploited to test other commercially available WTA protocols that use template-switching chemistries to amplify full-length RNA molecules and that use MDA-based cDNA amplification. Once all of the above have been performed to achieve and validate a microfluidic WTA protocol with low-representational bias, it will be applied to single cells. This tool can be used for the transcriptional profiling of hundreds of cells in a biological system of interest, a currently intractable proposition in conventional formats, for such applications as the elucidation of transcriptional mechanisms responsible for stem cell differentiation and renewal or the discovery of post-transcriptional modifications that play a role in human disease.

### Operation

While specific embodiments of the invention have been described and illustrated, such embodiments should be considered illustrative of the invention only and not as limiting the invention as construed in accordance with the accompanying claims.

### REFERENCES

1. Duffy D C, McDonald J C, Schueller O J A, & Whitesides G M (1998) Rapid Prototyping of Microfluidic Systems in Poly(dimethylsiloxane). Analytical Chemistry 70(23):4974-4984.
2. Unger M A, Chou H-P, Thorsen T, Scherer A, & Quake S R (2000) Monolithic Microfabricated Valves and Pumps by Multilayer Soft Lithography. Science 288(5463):113-116.
3. Thorsen T, Maerkl S J, & Quake S R (2002) Microfluidic Large-Scale Integration. Science 298(5593):580-584.
4. Hansen C L, Sommer M O A, & Quake S R (2004) Systematic investigation of protein phase behavior with a microfluidic formulator. Proceedings of the National Academy of Sciences of the United States of America 101(40):14431-14436.
5. Lau B T C, Baitz C A, Dong X P, & Hansen C L (2007) A Complete Microfluidic Screening Platform for Rational Protein Crystallization. Journal of the American Chemical Society 129(3):454-455.
6. Maerkl S J & Quake S R (2007) A Systems Approach to Measuring the Binding Energy Landscapes of Transcription Factors. Science 315(5809):233-237.
7. Singhal A, Haynes C A, & Hansen C L (Microfluidic Measurement of Antibody, àíAntigen Binding Kinetics from Low-Abundance Samples and Single Cells. Analytical Chemistry 82(20):8671-8679.
8. Ottesen E A, Hong J W, Quake S R, & Leadbetter J R (2006) Microfluidic Digital PCR Enables Multigene Analysis of Individual Environmental Bacteria. Science 314(5804):1464-1467.
9. Marcy Y, et al. (2007) Dissecting biological “dark matter” with single-cell genetic analysis of rare and uncultivated TM7 microbes from the human mouth. Proceedings of the National Academy of Sciences of the United States of America 104(29):11889-11894.
10. Fan H C, Wang J, Potanina A, & Quake S R (Whole-genome molecular haplotyping of single cells. Nat Biotech 29(1):51-57.
11. White A K, et al. (High-throughput microfluidic single-cell RT-qPCR. Proceedings of the National Academy of Sciences.
12. Balagadde F K, You L, Hansen C L, Arnold F H, & Quake S R (2005) Long-Term Monitoring of Bacteria Undergoing Programmed Population Control in a Microchemostat. Science 309(5731):137-140.
13. Taylor R J, et al. (2009) Dynamic analysis of MAPK signaling using a high-throughput microfluidic single-cell imaging platform. Proceedings of the National Academy of Sciences of the United States of America 106(10):3758-3763.
14. Fan H C & Quake S R (2007) Detection of Aneuploidy with Digital Polymerase Chain Reaction. Analytical Chemistry 79(19):7576-7579.
15. Clausell-Tormos J, et al. (2008) Droplet-Based Microfluidic Platforms for the Encapsulation and Screening of Mammalian Cells and Multicellular Organisms. Chemistry & Biology 15(5):427-437.
16. Brouzes E, et al. (2009) Droplet microfluidic technology for single-cell high-throughput screening. Proceedings of the National Academy of Sciences of the United States of America 106(34):14195-14200.
17. Agresti J J, et al. (2010) Ultrahigh-throughput screening in drop-based microfluidics for directed evolution. Proceedings of the National Academy of Sciences 107(9):4004-4009.
18. Tewhey R, et al. (2009) Microdroplet-based PCR enrichment for large-scale targeted sequencing. Nat Biotech 27(11):1025-1031.
19. Wheeler A R (2008) CHEMISTRY: Putting Electrowetting to Work. Science 322(5901):539-540.
20. Jensen E C, Bhat B P, & Mathies R A (A digital microfluidic platform for the automation of quantitative biomolecular assays. Lab on a Chip 10(6):685-691.
21. Chang M-P & Maharbiz M M (2009) Electrostatically-driven elastomer components for user-reconfigurable high density microfluidics. Lab on a Chip 9(9):1274-1281.
22. Fidalgo L M & Maerkl S J (A software-programmable microfluidic device for automated biology. Lab on a Chip 11(9):1612-1619.
23. Johnson Rulon E & Dettre Robert H (1964) Contact Angle Hysteresis. Contact Angle, Wettability, and Adhesion, Advances in Chemistry, (AMERICAN CHEMICAL SOCIETY), Vol 43, pp 112-135.
24. Chen J Z, Troian S M, Darhuber A A, & Wagner S (2005) Effect of contact angle hysteresis on thermocapillary droplet actuation. Journal of Applied Physics 97(1):014906.Helen Song D L C, Rustem F. Ismagilov, (2006) Reactions in Droplets in Microfluidic Channels. Angewandte Chemie International Edition 45(44):7336-7356.
25. Tice J D, Song H, Lyon A D, & Ismagilov R F (2003) Formation of Droplets and Mixing in Multiphase Microfluidics at Low Values of the Reynolds and the Capillary Numbers. Langmuir 19(22):9127-9133.
26. Fidalgo L M, Abell C, & Huck W T S (2007) Surface-induced droplet fusion in microfluidic devices. Lab on a Chip 7(8):984-986.
27. Bretherton F P (1961) The motion of long bubbles in tubes. Journal of Fluid Mechanics 10(02):166-188.
28. Baldessari F, Homsy G M, & Leal L G (2007) Linear stability of a draining film squeezed between two approaching droplets. Journal of Colloid and Interface Science 307(1):188-202.
29. Steinhaus B, Spicer P T, & Shen A Q (2006) Droplet Size Effects on Film Drainage between Droplet and Substrate. Langmuir 22(12):5308-5313.
30. Thorsen T, Roberts R W, Arnold F H, & Quake S R (2001) Dynamic Pattern Formation in a Vesicle-Generating Microfluidic Device. Physical Review Letters 86(18):4163-4166.
31. Niu X, Gulati S, Edel J B, & deMello A J (2008) Pillar-induced droplet merging in microfluidic circuits. Lab on a Chip 8(11):1837-1841.)
32. Shim J-u, et al. (2007) Control and Measurement of the Phase Behavior of Aqueous Solutions Using Microfluidics. Journal of the American Chemical Society 129(28):8825-8835.
33. Holtze C, et al. (2008) Biocompatible surfactants for water-in-fluorocarbon emulsions. Lab on a Chip 8(10):1632-1639
34. Lau B T C, Baitz C A, Dong X P, & Hansen C L (2007) A Complete Microfluidic Screening Platform for Rational Protein Crystallization. J. Am. Chem. Soc. 129(3):454-455.
35. Urbanski J P, Thies W, Rhodes C, Amarasinghe S, & Thorsen T (2006) Digital microfluidics using soft lithography. Lab on a Chip 6(1):96-104.
36. Hua Z, et al. (2006) A versatile microreactor platform featuring a chemical-resistant microvalve array for addressable multiplex syntheses and assays. Journal of Micromechanics and Microengineering 16(8): 1433-1443.
37. Lee C, Lee S, Shin S, & Hwang S (2008) Real-time PCR determination of rRNA gene copy number: absolute and relative quantification assays with &lt;i&gt;Escherichia coli&lt;/i&gt. Appl. Microbiol. Biotechnol. 78(2):371-376.
38. Marcy Y, et al. (2007) Nanoliter reactors improve multiple displacement amplification of genomes from single cells. Plos Genetics 3(9):1702-1708.
39. Zeng Y, Novak R, Shuga J, Smith M T, & Mathies R A (2010) High-Performance Single Cell Genetic Analysis Using Microfluidic Emulsion Generator Arrays. Analytical Chemistry 82(8):3183-3190.
40. Baret J-C, et al. (2009) Fluorescence-activated droplet sorting (FADS): efficient microfluidic cell sorting based on enzymatic activity. Lab on a Chip.
41. Wray C, Sojka W J (1978) Experimental Salmonella typhimurium infection in calves. Res Vet Sci 25: 139-143.
42. Fidalgo L M, et al. (2007) Surface-induced droplet fusion in microfluidic devices. Lab on a Chip, 2007, 7, 984-986

### ADDITIONAL REFERENCES

Ahn K, et al. (2006) Dielectrophoretic manipulation of drops for high-speed microfluidic sorting devices. Applied Physics Letters 88(2):024104.
Anna S L, Bontoux N, & Stone H A (2003) Formation of dispersions using “flow focusing” in microchannels. Applied Physics Letters 82(3):364-366.
Huebner A, et al. (2009) Static microdroplet arrays: a microfluidic device for droplet trapping, incubation and release for enzymatic and cell-based assays. Lab on a Chip 9(5):692-698. Link D R, et al. (2006) Electric Control of Droplets in Microfluidic Devices. Angewandte Chemie International Edition 45(16):2556-2560.
Luk V N, Mo G C H, & Wheeler A R (2008) Pluronic Additives: A Solution to Sticky Problems in Digital Microfluidics. Langmuir 24(12):6382-6389.
Niu X, Gielen F, deMello A J, & Edel J B (2009) Electro-Coalescence of Digitally Controlled Droplets. Analytical Chemistry 81(17):7321-7325.
Schmitz C H J, Rowat A C, Koster S, & Weitz D A (2009) Dropspots: a picoliter array in a microfluidic device. Lab on a Chip 9(1):44-49.
Song H, Tice J D, & Ismagilov R F (2003) A Microfluidic System for Controlling Reaction Networks in Time. Angewandte Chemie International Edition 42(7):768-772.
Wang W, Yang C, & Li C M (2009) On-demand microfluidic droplet trapping and fusion for on-chip static droplet assays. Lab on a Chip 9(11):1504-1506.

What is claimed is:

1. A method of determining a first position at which a dispersed phase droplet wets a surface of a channel having a uniform wettability, the method comprising:
(a) immersing the dispersed phase droplet in a continuous phase fluid, wherein the continuous phase fluid is immiscible with the dispersed phase droplet;
(b) flowing the dispersed phase droplet in the continuous phase through the channel at a dispersed phase droplet velocity, wherein the dispersed phase droplet is separated from the surface by a film of the continuous phase fluid having a film thickness; and
(c) rupturing the film at the first position, wherein the droplet wets the surface at the first position.

2. A method of determining a first position at which a dispersed phase droplet wets a surface of a channel, the method comprising:
(a) immersing the dispersed phase droplet in a continuous phase fluid, wherein the continuous phase fluid is immiscible with the dispersed phase droplet;
(b) flowing the dispersed phase in the continuous phase through the channel at a dispersed phase droplet velocity, wherein the dispersed phase droplet is separated from the surface by a film of the continuous phase fluid having a film thickness; and
(c) reducing the film thickness to rupture the film at the first position, wherein the droplet wets the surface at the first position.

3. The method of claim 1 or claim 2, wherein rupturing the film includes reducing the dispersed phase droplet velocity to reduce the film thickness.

4. The method of claim 2 or claim 3, wherein reducing the film thickness further includes removing a portion of the continuous phase fluid from the channel as the dispersed phase droplet approaches the first position.

5. A method of combining a plurality of dispersed phase droplets, the method comprising:
a. maintaining a first dispersed phase droplet wetted to a surface of a channel at a first position,
b. causing a second dispersed phase droplet to wet the surface of the channel at the first position according to the method of any one of claims 1 to 4, and
c. contacting the first dispersed phase droplet with the second dispersed phase droplet for a period sufficient for the first dispersed phase droplet and the second dispersed phase droplet to combine.

6. A method of removing a first portion of a dispersed phase immersed in a continuous phase fluid, wherein the continuous phase fluid is immiscible with the dispersed phase, from a dispersed phase retaining chamber operably configured to retain the portion provided that the volume of the first portion is less than the volume of the chamber, the method comprising:
a. immersing one or more dispersed phase droplets in the continuous phase fluid, to form a second portion of the dispersed phase;
b. flowing the second portion of the dispersed phase into the dispersed phase retaining chamber, wherein the total volume of the dispersed phase portions exceeds the volume of the dispersed phase retaining chamber,
c. contacting the first dispersed phase portion with the second dispersed phase portion for a period sufficient for the first dispersed portion and second dispersed phase portion to combine to form an elution stream encapsulated in the continuous phase fluid, and
d. flowing the elution stream through a dispersed phase retaining chamber exit.

7. A microfluidic device for reducing the thickness of a film of a continuous phase fluid encapsulating a dispersed phase droplet, wherein the dispersed phase droplet is immiscible in the continuous phase fluid, the device comprising:
(a) a channel for flowing the dispersed phase droplet; and
(b) a series of sieve elements operably configured to divert a portion of the continuous phase fluid from the channel to reduce the thickness of the film, wherein each sieve element has a diameter smaller than the diameter of the dispersed phase droplet.

8. A microfluidic device for reducing a velocity of a dispersed phase droplet encapsulated in a continuous phase fluid, wherein the dispersed phase droplet is immiscible with the continuous phase fluid, the device comprising:
(a) a channel for flowing the dispersed phase droplet; and
(b) a series of sieve elements operably configured to permanently divert a portion of the continuous phase fluid from the channel to reduce the velocity of the dispersed phase droplet, wherein each sieve element has a diameter smaller than the diameter of the dispersed phase droplet.

9. The microfluidic device of claim 7 or 8, wherein the sieve elements are generally perpendicular to the channel.

10. The microfluidic device of any one of claims 7 to 9, further comprising a dispersed phase retaining chamber in fluid communication with the channel for receiving the dispersed phase droplet.

11. The microfluidic device of claim 10, wherein the sieve elements are operably configured to divert the portion of the continuous phase fluid from the channel prior to reaching the dispersed phase retaining chamber.

12. The microfluidic device of claim 10 or 11, wherein the microfluidic device further includes a bypass channel in fluid communication with the series of sieve elements, wherein the bypass channel is operably configured to receive the portion and maintain the portion outside the storage chamber.

13. A process of treating a dispersed phase droplet in a microfluidic device, the process comprising:
(a) immersing a first dispersed phase droplet in a continuous phase fluid, wherein the continuous phase fluid is immiscible with the first dispersed phase droplet, to form a first portion of a dispersed phase;
(b) flowing the first dispersed phase droplet into a storage element of the device with a first dispersed phase droplet velocity, wherein the storage element comprises
a main channel and a dispersed phase retaining chamber for receiving said dispersed phase droplet from the main channel, wherein the main channel is operably configured to reduce dispersed phase droplet velocity as said dispersed phase droplet approaches the retaining chamber, and wherein the retaining chamber is operably configured to retain said dispersed phase droplet within the storage element provided that the total volume of the dispersed phase within the retaining chamber is less than the volume of the retaining chamber,
wherein the first dispersed phase droplet is separated from a surface of the storage element by a first film of the continuous phase fluid having a first film thickness; and
(c) rupturing the first film at a first position within the storage element, wherein the first dispersed phase droplet wets the surface at the first position.

14. The process of claim 13, wherein rupturing the first film at the first position comprises reducing the first film thickness to rupture the first film at the first position.

15. The process of claim 14, wherein reducing the first film thickness comprises reducing the first dispersed phase droplet velocity.

16. The process of any one of claims 13 to 15, wherein the surface has a uniform wettability.

17. The process of any one of claims 13 to 16, further comprising:
(a) immersing a second dispersed phase droplet in the continuous phase fluid, wherein the continuous phase fluid is immiscible with the second dispersed phase droplet, to form a second portion of the dispersed phase;
(b) flowing the second dispersed phase droplet into the storage element with a second dispersed phase droplet velocity, wherein the second dispersed phase droplet is separated from the surface of the storage element by a second film of the continuous phase fluid having a second film thickness; and
(c) rupturing the second film at a second position within the storage element, wherein the second droplet wets the surface at the second position.

18. The process of claim 16, wherein the first position is substantially the same as the second position, and wherein the process further comprises contacting the first dispersed phase droplet with the second dispersed phase droplet for a period sufficient for first dispersed phase droplet and second dispersed phase droplet to combine.

19. The process of claim 17, further comprising:
(a) immersing a third dispersed phase droplet in the continuous phase fluid, wherein the continuous phase fluid is immiscible with the third dispersed phase droplet, to form a third portion of the dispersed phase;
(b) flowing the third dispersed phase droplet into the storage element with a third dispersed phase droplet velocity, wherein the third dispersed phase droplet is separated from the surface of the storage element by a third film of the continuous phase fluid having a third film thickness; and
(c) rupturing the third film at a third position within the storage element, wherein the third droplet wets the surface at the third position.

20. The process of claim 19, wherein:
(a) the third position is substantially the same as the first position, and wherein the process further comprises contacting the third dispersed phase droplet with the first dispersed phase droplet for a period sufficient for first dispersed phase droplet and third dispersed phase droplet to combine; or
(b) the third position is substantially the same as the second position, and wherein the process further comprises contacting the third dispersed phase droplet with the second dispersed phase droplet for a period sufficient for second dispersed phase droplet and third dispersed phase droplet to combine.

21. The process of claim 19, wherein the third position lies between the first position and the second position.

22. The process of claim 19, wherein the third position is substantially close to the first position and to the second position, and wherein the process further comprises contacting the third dispersed phase droplet with both the first dispersed phase droplet and the second dispersed phase droplet for a period sufficient for the third dispersed phase droplet to combine with the first dispersed phase droplet and the second dispersed phase droplet.

23. The process of any one of claims 13 to 22, further comprising:
(a) immersing a fourth dispersed phase droplet in the continuous phase fluid, wherein the continuous phase fluid is immiscible with the fourth dispersed phase droplet, to form a fourth portion of the dispersed phase;
(b) flowing the fourth dispersed phase droplet into the dispersed phase retaining chamber, wherein the total volume of the dispersed phase within the storage element exceeds the volume of the dispersed phase retaining chamber;
(c) contacting the dispersed phase droplets within storage element with the fourth dispersed phase droplet for a period sufficient for the fourth dispersed phase droplet and the dispersed phase droplets to combine to form an elution stream encapsulated in the carrier fluid; and
(d) flowing the elution stream through a dispersed phase retaining chamber exit.

24. A microfluidic system for storing and processing dispersed phase droplets, the system comprising:
(a) an array of at least two parallel independently addressable storage elements, wherein each storage element comprises:
a main channel and a dispersed phase retaining chamber for receiving at least one of said dispersed phase droplets from the main channel, wherein the at least one of said dispersed phase droplets forms a portion of a dispersed phase within the storage element, and wherein the main channel is operably configured to reduce the velocity of the at least one of said dispersed phase droplets as the at least one of said dispersed phase droplets approaches the dispersed phase retaining chamber, and wherein the dispersed phase retaining chamber is operably configured to retain the at least one of said dispersed phase droplets within the storage element provided that the total volume of the dispersed phase within the dispersed phase retaining chamber is less than the volume of the retaining chamber;
(b) an inlet channel shared by the at least two storage elements for flowing the at least one of said dispersed phase droplets to a selected storage element; and
(c) an elution channel shared by the at least two storage elements for flowing the dispersed phase from the selected storage element.

25. A method of determining a first position at which a dispersed phase droplet wets a dispersed phase wetting surface of a microfluidic device, the wetting surface having a uniform wettability, the method comprising:
(a) immersing the dispersed phase droplet in a continuous phase fluid, wherein the continuous phase fluid is immiscible with the dispersed phase droplet;
(b) flowing the dispersed phase droplet immersed through the microfluidic device at a dispersed phase droplet velocity, wherein the dispersed phase droplet is separated from the surface of the conduit by a film of the carrier liquid having a film thickness; and
(c) reducing the film thickness to rupture the film at the first position.
